(19) United States
(12) Patent Application Publication
(10) Pub. No.: US 2015/0181879 A1
(43) Pub. Date: Jul. 2, 2015

(54) Substituted pyridine compounds having herbicidal activity

(71) Applicant: BASF SE, Ludwigshafen (DE)

(72) Inventors: Matthias Witschel, Bad Duerkheim (DE); Helmut Kraus, Wissembourg (FR); Johannes Hutzler, Waldsee (DE); Trevor William Newton, Neustadt (DE); Ruediger Reingruber, Ludwigshafen (DE); Timo Frassetto, Mannheim (DE); Liliana Parra Rapado, Offenburg (DE); Gilbert Besong, Bad Duerkheim (DE); Michael Rack, Eppelheim (DE); Andree van der Kloet, Arnhem (NL); Thomas Seitz, Viernheim (DE); Jens Lerchl, Golm (DE); Klaus Kreuz, Denzlingen (DE); Maciej Pasternak, Ludwigshafen (DE); Richard Roger Evans, Limburgerhof (DE)

(21) Appl. No.: 14/403,723

(22) PCT Filed: May 27, 2013
(86) PCT No.: PCT/EP2013/060866

§ 371 Date: Nov. 25, 2014

Related U.S. Application Data
(60) Provisional application No. 61/654,122, filed on Jun. 1, 2012.

(30) Foreign Application Priority Data
Jun. 1, 2012 (EP) 12170487.8

(51) Int. Cl. A01N 43/90; C07D 495/04

(52) U.S. Cl. CPC A01N 43/90; C07D 495/04

(57) ABSTRACT

The present invention provides a substituted pyridine compound of the formula I

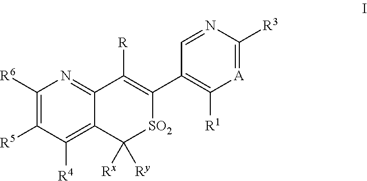

or an agriculturally suitable salt or N-oxide thereof, wherein the variables in the formula I are defined as in the description. Substituted pyridine compounds of formula I are useful as herbicides.

The present invention relates to substituted pyridine compounds of the general formula I defined below and to their use as herbicides. Moreover, the invention relates to compositions for crop protection and to a method for controlling unwanted vegetation.

WO 2009/063180 and WO 2010/029311 describe certain herbicidal pyridopyrazines.

WO 2010/130970 describes certain 6,6-dioxo-6-thia-1,4-diaza-naphthalene derivatives having herbicidal activity.

WO 2012/084755 and WO 2011/117152 describe certain substituted pyridine derivatives having herbicidal activity.

However, the herbicidal properties of these known compounds with regard to the harmful plants are not always entirely satisfactory.

It is therefore an object of the present invention to provide compounds having improved herbicidal action. To be provided are in particular compounds which have high herbicidal activity, in particular even at low application rates, and which are sufficiently compatible with crop plants for commercial utilization.

These and further objects are achieved by substituted pyridine compound of the formula I, defined below, and by their agriculturally suitable salts or N-oxides.

Accordingly, the present invention provides a substituted pyridine compound of the formula I

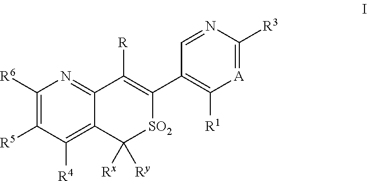

wherein
R is hydroxy or O—R^{A}, where R^{A} is C₁-C₈-alkyl, C₂-C₈-alkenyl, C₂-C₈-alkynyl, C₁-C₈-alkylcarbonyl, C₁-C₈-alkoxycarbonyl, C₁-C₈-alkylthiocarbonyl or C₁-C₈-alkylsulfonyl, where the aryl moiety is unsubstituted or substituted by one to five R^{a} and each R^{a} is independently halogen, cyano, nitro, C₁-C₈-alkyl, C₁-C₈-haloalkyl, C₁-C₈-alkoxy or C₁-C₈-haloalkoxy;
R¹ is cyano, halogen, nitro, C₁-C₆-alkyl, C₃-C₆-cycloalkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, C₂-C₆-alkenyloxy, C₂-C₆-alkynyloxy, C₁-C₆-haloalkoxy, C₁-C₄-haloalkoxy-C₁-C₄-alkoxy, S(O)ₙR^{b}, Z-phenoxy or Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};
   Z is independently a covalent bond or C₁-C₄-alkylene;
   N is independently 0, 1 or 2;
   R^{b} is independently C₁-C₈-alkyl, C₂-C₆-alkenyl, C₂-C₆-alkynyl, C₂-C₆-haloalkenyl, C₂-C₆-haloalkynyl or C₁-C₆-haloalkyl;
   R^{c} is independently Z—CN, Z—OH, Z—NO₂, Z-halogen, oxo (═O), ═N—R^{d}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{d}, NRⁱRⁱⁱ, Z-phenyl or S(O)ₙR^{b}; or two groups R^{c} may together form a ring which has 3 to 6 ring members and, in addition to carbon atoms, may contain heteroatoms selected from the group consisting of O, N and S and may be unsubstituted or substituted by further groups R^{c};
   R^{d} is independently hydrogen, OH, C₁-C₈-alkyl, C₁-C₄-haloalkyl, Z—C₃-C₆-cycloalkyl, C₂-C₈-alkenyl, Z—C₅-C₆-cycloalkenyl, C₂-C₈-alkynyl, Z—C₁-C₆-alkoxy, Z—C₁-C₄-haloalkoxy, Z—C₃-C₈-alkenyloxy, Z—C₃-C₈-alkynyloxy, NRⁱRⁱⁱ, C₁-C₆-alkylsulfonyl, Z-(tri-C₁-C₄-alkyl)silyl, Z-phenyl, Z-phenoxy, Z-phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{c};
   Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, Z—C₁-C₈-haloalkoxy, Z—C(═O)—R^{d}, Z-phenyl, a 3- to 7-membered monocyclic or 9- or 10-membered bicyclic saturated, unsaturated or aromatic heterocycle which contains 1, 2, 3 or 4 heteroatoms selected from the group consisting of O, N and S and which is attached via Z;
   Rⁱ and Rⁱⁱ together with the nitrogen atom to which they are attached may also form a 5- or 6-membered monocyclic or 9- or 10-membered bicyclic heterocycle which contains 1, 2, 3 or 4 heteroatoms selected from the group consisting of O, N and S;
A is N or C—R²;
R²,R³ independently of one another are hydrogen, Z-halogen, Z—CN, Z—OH, Z—NO₂, C₁-C₈-alkyl, C₁-C₄-haloalkyl, C₂-C₈-alkenyl, C₂-C₈-alkynyl, C₂-C₈-haloalkenyl, C₂-C₈-haloalkynyl, Z—C₁-C₈-alkoxy, Z—C₁-C₈-haloalkoxy, Z—C₁-C₄-alkoxy-C₁-C₄-alkoxy, Z—C₁-C₄-alkythio, Z—C₁-C₄-alkylthio-C₁-C₄-alkylthio, Z—C₁-C₆-haloalkylthio, C₂-C₆-alkenyloxy, C₂-C₆-alkynyloxy, C₁-C₆-haloalkoxy, C₁-C₄-haloalkoxy-C₁-C₄-alkoxy, Z—C₃-C₁₀-cycloalkyl, O—Z—C₃-C₁₀-cycloalkyl, Z—C(═O)—R^{d}, NRⁱRⁱⁱ, Z—NRⁱSO₂Rⁱⁱ, S(O),R^{b}, Z-phenyl, Z¹-phenyl, Z-heterocyclyl or Z¹-heterocyclyl, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};

R² together with the group attached to the adjacent carbon atom may also form a 5- to 10-membered saturated or partially or fully unsaturated mono- or bicyclic ring which, in addition to carbon atoms, may contain 1, 2 or 3 heteroatoms selected from the group consisting of O, N and S and may be substituted by further groups R^{c};
Z¹ is independently a covalent bond, C₁-C₄-alkyleneoxy, C₁-C₄-oxyalkylene or C₁-C₄-alkyleneoxy-C₁-C₄-alkylene;
R⁴,R⁵,R⁶ independently of one another are hydrogen, halogen or C₁-C₄-alkyl;
R^{x}, R^{y} independently of one another are hydrogen, C₁-C₅-alkyl, C₂-C₅-alkenyl, C₂-C₅-alkynyl, C₁-C₅-haloalkyl, C₁-C₂-alkoxy-C₁-C₂-alkyl or halogen; or R^{x} and R^{y} are together a C₂-C₅-alkylene or C₂-C₅-alkenylene chain and form a 3-, 4-, 5- or 6-membered saturated, partially unsaturated or fully unsaturated monocyclic ring together with the carbon atom they are bonded to, wherein 1 or 2 of any of the CH₂ or CH groups in the C₂-C₅-alkylene or C₂-C₅-alkenylene chain may be replaced by 1 or 2 heteroatoms independently selected from O or S;
   where in the groups R¹, R² and R³ and their subsubstituents, the carbon chains and/or the cyclic groups may be partially or fully substituted by groups R^{c},
   or an agriculturally suitable salt or N-oxide thereof.

The present invention also provides the use of substituted pyridine compounds of formula I as herbicides, i.e. for controlling harmful plants.

The present invention also provides agrochemical compositions comprising at least one substituted pyridine compound of formula I and auxiliaries customary for formulating crop protection agents.

The present invention furthermore provides a method for controlling unwanted vegetation where a herbicidally effective amount of at least one substituted pyridine compound of formula I is allowed to act on plants, their seeds and/or their habitat. Application can be done before, during and/or after, preferably during and/or after, the emergence of the undesirable plants.

Moreover, the invention relates to processes and intermediates for preparing the substituted pyridine compound of formula I.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

As used herein, the terms “controlling” and “combating” are synonyms.

As used herein, the terms “undesirable vegetation” and “harmful plants” are synonyms.

If the substituted pyridine compounds of formula I as described herein are capable of forming geometrical isomers, for example E/Z isomers, it is possible to use both, the pure isomers and mixtures thereof, in the compositions according to the invention.

If the substituted pyridine compounds of formula I as described herein have one or more centers of chirality and, as a consequence, are present as enantiomers or diastereomers, it is possible to use both, the pure enantiomers and diastereomers and their mixtures, in the compositions according to the invention.

If the substituted pyridine compounds of formula I as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

The substituted pyridine compounds of formula I as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

The organic moieties mentioned in the definition of the variables R, R¹, R², R³, R⁴, R⁵, R⁶, R^{x}, R^{y}, R^{A}, R^{a}R^{b}, R^{c}, R^{d}, Rⁱ, Rⁱⁱ, Z and Z¹ are—like the term halogen—collective terms for individual enumerations of the individual group members. The term halogen denotes in each case fluorine, chlorine, bromine or iodine. All hydrocarbon chains, i.e. all alkyl, can be straight-chain or branched, the prefix Cₙ-Cₘ denoting in each case the possible number of carbon atoms in the group.

Examples of such meanings are:
C₁-C₂-alkyl and also the C₁-C₂-alkyl moieties of C₁-C₂-alkoxy-C₁-C₂-alkyl includes CH₃ and C₂H₅;
C₁-C₄-alkyl and also the C₁-C₄-alkyl moieties of Z-(tri-C₁-C₄-alkyl)silyl, aryl-C₁-C₄-alkyl and C₃-C₆-cycloalkyl-C₁-C₄-alkyl; C₁-C₂-alkyl as mentioned above, and also, for example, n-propyl, CH(CH₃)₂, n-butyl, CH(CH₃)—C₂H₅, CH₂—CH(CH₃)₂ and C(CH₃)₃;
C₁-C₅-alkyl: C₁-C₄-alkyl as mentioned above, and also, for example, n-pentyl, 1-methylbutyl, 2-methylbutyl, 3-methylbutyl, 2,2-dimethylpropyl, 1,1-dimethylpropyl, 1,2-dimethylpropyl, and 1-ethylpropyl;
C₁-C₆-alkyl and also the C₁-C₆-alkyl moieties of C₁-C₆-alkylsulfonyl and C₁-C₆-alkyoxy-C₁-C₆-alkyl: C₁-C₅-alkyl as mentioned above, and also, for example, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;
C₁-C₈-alkyl and also the C₁-C₈-alkyl moieties of C₁-C₈-alkylcarbonyl, C₁-C₈-alkylthiocarbonyl and C₁-C₈-alkylsulfonyl: C₁-C₆-alkyl as mentioned above, and also, for example, n-heptyl, n-octyl or 2-ethylhexyl;
C₁-C₄-haloalkyl: a C₁-C₄-alkyl radical as mentioned above which is partially or fully substituted by fluorine, chlorine, bromine and/or iodine, for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2,tetrafluoroethyl and 1-trifluoromethyl-1,2,2,2-tetrafluoroethyl;
C₁-C₅-haloalkyl: C₁-C₄-haloalkyl as mentioned above, and also, for example, 5-fluoropentyl, 5-chloropentyl, 5-bromopentyl, 5-iodopentyl and undecafluoropentyl;
C₁-C₆-haloalkyl: C₁-C₅-haloalkyl as mentioned above, and also, for example, 6-fluorohexyl, 6-chlorohexyl, 6-bromohexyl, 6-iodohexyl and dodecafluorohexyl;
C₃-C₆-cycloalkyl and also the cycloalkyl moieties of Z—C₃-C₆-cycloalkyl, —O—C₃-C₆-cycloalkyl and C₃-C₆-cycloalkyl-C₁-C₄-alkyl: monocyclic saturated hydrocarbons having 3 to 6 ring members, such as cyclopropyl, cyclobutyl, cyclopentyl and cyclohexyl;
C₃-C₁₀-cycloalkyl and also the cycloalkyl moieties of Z—C₃-C₁₀-cycloalkyl and O—Z—C₃-C₁₀-cycloalkyl: C₃-C₆-cycloalkyl as mentioned above, and also, for example, cycloheptyl, cyclooctyl, cyclononyl and cyclodecyl;
C₂-C₅-alkenyl: for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl and 1-ethyl-2-propenyl;
C₂-C₆-alkenyl and also the alkenyl moieties of C₂-C₆-alkenyloxy: C₂-C₅-alkenyl as mentioned above, and also, for example, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₂-C₈-alkenyl: C₂-C₆-alkenyl as mentioned above, and also, for example 1-heptenyl, 2-heptenyl, 3-heptenyl, 1-octenyl, 2-octenyl, 3-octenyl and 4-octenyl;
C₃-C₈-alkenyl and also the alkenyl moieties of Z—C₃-C₈-alkenyloxy: C₂-C₈-alkenyl as mentioned above, with the exception of ethenyl;
C₃-C₆-cycloalkenyl and also the cycloalkenyl moieties of Z—C₃-C₆-cycloalkenyl: for example cyclopropenyl, cyclobutenyl, cyclopentenyl and cyclohexenyl;
C₅-C₆-cycloalkenyl and also the cycloalkenyl moieties of Z—C₅-C₆-cycloalkenyl: for example cyclopentenyl and cyclohexenyl;
C₂-C₆-haloalkenyl: a C₂-C₆-alkenyl radical as mentioned above which is partially or fully substituted by fluorine, chlorine, bromine and/or iodine, for example 2-dibromoethenyl, 2-fluoro-2-bromoethenyl, 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₃-C₆-haloalkenyl: C₂-C₆-haloalkenyl as mentioned above with the exception of C₂-haloalkenyl radicals;
C₂-C₈-haloalkenyl: C₂-C₆-haloalkenyl as mentioned above, and also, for example, 3-fluoron-heptenyl-1,1,3,3,-trichloro-n-heptenyl-5 and 1,3,5-trichloro-n-octenyl-6;
C₂-C₅-alkynyl: for example ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl and 1-ethyl-2-propynyl;
C₂-C₆-alkynyl and also the alkynyl moieties of C₂-C₆-alkynyloxy: C₂-C₅-alkynyl as mentioned above, and also, for example, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₂-C₈-alkynyl: C₂-C₆-alkynyl as mentioned above, and also, for example, 1-heptynyl, 2-heptynyl, 1-octynyl and 2-octynyl;
C₃-C₈-alkynyl and also the alkynyl moieties of Z—C₃-C₈-alkynyloxy: a C₂-C₈-alkynyl radical as mentioned above with the exception of C₂-alkynyl radicals;
C₂-C₆-haloalkynyl: a C₂-C₆-alkynyl radical as mentioned above which is partially or fully substituted by fluorine, chlorine, bromine and/or iodine, for example 1,1-difluoroprop-2-yn-1-yl, 3-chloroprop-2-yn-1-yl, 3-bromoprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₃-C₆-haloalkynyl: a C₂-C₆-haloalkynyl as mentioned above with the exception of C₂-haloalkynyl radicals;
C₂-C₈-haloalkynyl: C₂-C₆-haloalkynyl as mentioned above, and also, for example, 1-chloro-2-heptynyl and 1-chloro-2-octynyl;
C₁-C₂-alkoxy and also the C₁-C₂-alkoxy moieties of C₁-C₂-alkoxy-C₁-C₂-alkyl: for example methoxy and ethoxy;
C₁-C₄-alkoxy and also the C₁-C₄-alkoxy moieties of Z—C₁-C₄-alkoxy-C₁-C₄-alkoxy, haloalkoxy-C₁-C₄-alkoxy, hydroxycarbonyl-C₁-C₄-alkoxy and C₁-C₆-alkoxycarbonyl-C₁-C₄-alkoxy: C₁-C₂-alkoxy as mentioned above, and also, for example, propoxy, 1-methylethoxy butoxy, 1-methylpropoxy, 2-methylpropoxy and 1,1-dimethylethoxy; C₁-C₆-alkoxy and also the C₁-C₆-alkoxy moieties of Z—C₁-C₆-alkoxy and C₁-C₆-alkoxycarbonyl-C₁-C₄-alkoxy: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;
C₁-C₈-alkoxy and also the C₁-C₈-alkoxy moieties of Z—C₁-C₈-alkoxy and C₁-C₈-alkoxycarbonyl: C₁-C₆-alkoxy as mentioned above, and also, for example, heptoxy, octoxy, 1,1,3,3-tetramethylbutoxy and 2-ethylhexoxy;
C₁-C₄-haloalkoxy and also the C₁-C₄-haloalkoxy moieties of Z—C₁-C₄-haloalkoxy and C₁-C₄-haloalkoxy-C₁-C₄-alkoxy: for example OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;
C₁-C₆-haloalkoxy: C₁-C₄-haloalkoxy as mentioned above, and also, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy;
C₁-C₈-haloalkoxy and also the C₁-C₈-haloalkoxy moieties of Z—C₁-C₈-haloalkoxy: C₁-C₆-haloalkoxy as mentioned above, and also, for example, 7-fluoroheptoxy, 7-chloroheptoxy, 7-bromoheptoxy, 7-iodoheptoxy, perfluoroheptoxy, 8-fluorooctoxy, 8-chlorooctoxy, 8-bromooctoxy, 8-iodooctoxy and octadecafluorooctoxy;
C₁-C₄-alkylthio also the C₁-C₄-alkylthio moieties of Z—C₁-C₄-alkylthio, Z—C₁-C₄-alkylthio-C₁-C₄-alkylthio: for example methylthio, ethylthio, propylthio, 1-methylethylthio, butylthio, 1-methylpropylthio, 2-methylpropylthio and 1,1-dimethylethylthio;
C₁-C₄-haloalkylthio: for example SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio;
C₁-C₆-haloalkylthio and also the C₁-C₆-alkylthio moieties of Z—C₁-C₆-haloalkylthio: C₁-C₄-haloalkylthio as mentioned above, and also, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio;
C₁-C₄-alkylene and also the C₁-C₄-alkylene moieties in C₁-C₄-alkyleneoxy, C₁-C₄-oxyalkylene and C₁-C₄-alkyleneoxy-C₁-C₄-alkylene: a straight carbon chain having from 1 to 4 carbon atoms and having only carbon-carbon single bonds, for example methylene (CH₂), ethylene (CH₂CH₂), n-propylene (CH₂CH₂CH₂) and n-butylene (CH₂CH₂CH₂CH₂);
C₂-C₅-alkylene: C₁-C₄-alkylene as mentioned above, and also n-pentylene (CH₂CH₂CH₂CH₂CH₂);
C₂-C₅-alkenylene chain: a straight carbon chain having from 2 to 5 carbon atoms and at least one carbon-carbon double bond and no carbon-carbon triple bond, for example, CH═CH, CH═CH—CH₂, CH═CH—CH₂CH₂, CH═CH—CH═CH₂ and CH═CH—CH₂CH₂CH₂;
a 3- to 7-membered monocyclic or 9- or 10-membered bicyclic saturated, unsaturated or aromatic heterocycle which contains 1, 2, 3 or 4 heteroatoms selected from the group consisting of O, N and S means, for example, pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, oxazol-2-yl, oxazol-4-yl, oxazol-5-yl, thiazol-2-yl, thiazol-4-yl, thiazol-5-yl, pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl; [2H]-tetrazol-5-yl, 2-tetrahydrofuryl, 3-tetrahydrofuryl, 2-oxetanyl and 3-oxetanyl;
a 5- or 6-membered monocyclic or 9- or 10-membered bicyclic heterocycle which contains 1, 2, 3 or 4 heteroatoms selected from the group consisting of O, N and S means, for example: pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, oxazol-2-yl, oxazol-4-yl, oxazol-5-yl, thiazol-2-yl, thiazol-4-yl and thiazol-5-yl, pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, 2-tetrahydrofuryl and 3-tetrahydrofuryl;
a 5- to 10-membered saturated or partially or fully unsaturated mono- or bicyclic ring which, in addition to carbon atoms, may contain 1, 2 or 3 heteroatoms selected from the group consisting of O, N and S means, for example: pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, oxazol-2-yl, oxazol-4-yl, oxazol-5-yl, thiazol-2-yl, thiazol-4-yl and thiazol-5-yl, pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, cyclopentyl, cyclohexyl, 2-tetrahydrofuryl and 3-tetrahydrofuryl.

The preferred embodiments of the invention mentioned herein below have to be understood as being preferred either independently from each other or in combination with one another.

Preferred are compounds of formula I wherein
R is hydroxy or O—R^{A}, where R^{A} is C₁-C₈-alkyl, C₂-C₈-alkenyl, C₂-C₈-alkynyl, C₁-C₈-alkylcarbonyl, C₁-C₈-alkoxycarbonyl, C₁-C₈-alkylthiocarbonyl or C₁-C₈-alkylsulfonyl, where the aryl moiety is unsubstituted or substituted by one to five R^{a} and each R^{a} is independently halogen, cyano, nitro, C₁-C₈-alkyl, C₁-C₈-alkoxy or C₁-C₈-haloalkoxy;
R¹ is cyano, halogen, nitro, C₁-C₆-alkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{b}, Z-phenoxy or Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};
   Z is independently a covalent bond or C₁-C₄-alkylene;
   n is independently 0, 1 or 2;
   R^{b} is independently C₁-C₈-alkyl, C₂-C₆-alkenyl, C₂-C₆-alkynyl, C₂-C₆-haloalkenyl, C₂-C₆-haloalkynyl or C₁-C₆-haloalkyl;
   R^{c} is independently Z—CN, Z—OH, Z—NO₂, Z-halogen, oxo (═O), ═N-R^{d}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{d}, NRⁱRⁱⁱ, Z-(tri-C₁-C₄-alkyl)silyl, Z-phenyl or S(O)ₙR^{b}; or two groups R^{c} may together form a ring which has 3 to 6 ring members and, in addition to carbon atoms, may contain heteroatoms selected from the group consisting of O, N and S and may be unsubstituted or substituted by further groups R^{c};
      R^{d} is independently hydrogen, OH, C₁-C₈-alkyl, C₁-C₄-haloalkyl, Z—C₃-C₆-cycloalkyl, C₂-C₈-alkenyl, Z—C₅-C₆-cycloalkenyl, C₂-C₈-alkynyl, Z—C₁-C₆-alkoxy, Z—C₁-C₄-haloalkoxy, Z—C₃-C₈-alkenyloxy, Z—C₃-C₈-alkynyloxy, NRⁱRⁱⁱ, C₁-C₆-alkylsulfonyl, Z-phenyl, Z-phenoxy, Z-phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{c};
         Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, Z—C₁-C₈-haloalkoxy, Z—C(═O)—R^{d}, Z-phenyl, a 3- to 7-membered monocyclic or 9- or 10-membered bicyclic saturated, unsaturated or aromatic heterocycle which contains 1, 2, 3 or 4 heteroatoms selected from the group consisting of O, N and S and which is attached via Z;
      Rⁱ and Rⁱⁱ together with the nitrogen atom to which they are attached may also form a 5- or 6-membered monocyclic or 9- or 10-membered bicyclic heterocycle which contains 1, 2, 3 or 4 heteroatoms selected from the group consisting of O, N and S;
A is N or C—R²;
R²,R³ independently of one another are hydrogen, Z-halogen, Z—CN, Z—OH, Z—NO₂, C₁-C₈-alkyl, C₁-C₄-haloalkyl, C₂-C₈-alkenyl, C₂-C₈-alkynyl, C₂-C₈-haloalkenyl, C₂-C₈-haloalkynyl, Z—C₁-C₈-alkoxy, Z—C₁-C₈-haloalkoxy, Z—C₁-C₄-alkoxy-C₁-C₄-alkoxy, Z—C₁-C₄-alkythio, Z—C₁-C₄-alkylthio-C₁-C₄-alkylthio, Z—C₁-C₆-haloalkylthio, C₂-C₆-alkenyloxy, C₂-C₆-alkynyloxy, C₁-C₆-haloalkoxy, C₁-C₄-haloalkoxy-C₁-C₄-alkoxy, Z—C₃-C₁₀-cycloalkyl, O—Z—C₃-C₁₀-cycloalkyl, Z—C(═O)—R^{d}, NRⁱRⁱⁱ, Z-(tri-C₁-C₄-alkyl)silyl, S(O)ₙR^{b}, Z-phenyl, Z¹-phenyl, Z-heterocyclyl or Z¹-heterocyclyl, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};

R² together with the group attached to the adjacent carbon atom may also form a 5- to 10-membered saturated or partially or fully unsaturated mono- or bicyclic ring which, in addition to carbon atoms, may contain 1, 2 or 3 heteroatoms selected from the group consisting of O, N and S and may be substituted by further groups R^{c};
Z¹ is independently a covalent bond, C₁-C₄-alkyleneoxy, C₁-C₄-oxyalkylene or C₁-C₄-alkyleneoxy-C₁-C₄-alkylene;
R⁴,R⁵,R⁶ independently of one another are hydrogen, halogen or C₁-C₄-alkyl;
R^{x}, R^{y} independently of one another are hydrogen, C₁-C₈-alkyl, C₂-C₅-alkenyl, C₂-C₅-alkynyl, C₁-C₈-haloalkyl, C₁-C₂-alkoxy-C₁-C₂-alkyl or halogen; or R^{x} and R^{y} are together a C₂-C₅-alkylene or C₂-C₅-alkenylene chain and form a 3-, 4-, 5- or 6-membered saturated, partially unsaturated or fully unsaturated monocyclic ring together with the carbon atom they are bonded to, wherein 1 or 2 of any of the CH₂ or CH groups in the C₂-C₅-alkylene or C₂-C₅-alkenylene chain may be replaced by 1 or 2 heteroatoms independently selected from O or S;
   where in the groups R¹, R² and R³ and their subsubstituents, the carbon chains and/or the cyclic groups may be partially or fully substituted by groups R^{c},
   or an agriculturally suitable salt or N-oxide thereof.

According to a preferred embodiment of the invention preference is also given to those compounds of formula I, wherein the variables, either independently of one another or in combination with one another, have the following meanings:

Preferably, R is hydroxy or O—R^{A}, where R^{A} is C₁-C₈-alkyl, C₂-C₈-alkenyl, C₂-C₈-alkynyl, aryl-C₁-C₄-alkyl, C₁-C₈-alkylcarbonyl, C₁-C₈-alkoxycarbonyl, C₁-C₈-alkylthiocarbonyl or C₁-C₈-alkylsulfonyl, where the aryl moiety is unsubstituted.

More preferably, R is hydroxy or O—R^{A}, where R^{A} is C₁-C₈-alkylcarbonyl.

In one embodiment, R is hydroxy.

In one embodiment, R is O—R^{A}, where R^{A} is C₁-C₈-alkylcarbonyl.

In another embodiment, R is selected from the group consisting of hydroxy, methoxy, allyloxy, propargyloxy, cyclopropylcarbonyloxy, benzyloxy, prop-2-ylcarbonyloxy, 2-methyl-prop-2-ylcarbonyloxy, methoxycarbonyloxy, ethoxycarbonyloxy, methylthiocarbonyloxy, ethylthiocarbonyloxy and methylsulfonyloxy.

In one embodiment, R is selected from the group consisting of hydroxy, methoxy, prop-2-ylcarbonyloxy, 2-methyl-prop-2-ylcarbonyloxy and methoxycarbonyloxy.

Preferably, R is selected from the group consisting of hydroxy, cyclopropylcarbonyloxy, and 2-methyl-prop-2-ylcarbonyloxy.

In particular, R is hydroxy or 2-methyl-prop-2-ylcarbonyloxy.

In one embodiment, R is hydroxy.

In one embodiment, R is 2-methyl-prop-2-ylcarbonyloxy.

Rⁱ and Rⁱⁱ are preferably C₁-C₈-alkyl, C₁-C₄-haloalkyl, Z—C₃-C₆-cycloalkyl, Z—C₁-C₈-alkoxy, Z—C₁-C₈-haloalkoxy, Z-phenyl, Z—C(═O)—R^{d} or Z-hetaryl. Preference is given here to CH₃, C₂H₅, n-propyl, CH(CH₃)₂, butyl, 2-choroethyl, cyclopentyl, cyclohexyl, 2-ethoxymethyl, 2-chloroethoxy, phenyl, pyrimidines or triazines, which rings are unsubstituted or substituted. Preferred substituents are C₁-C₄-alkylcarbonyl or C₁-C₄-haloalkylcarbonyl, in particular C(═O)—CH₃, C(═O)—C₂H₅, C(═O)—C₃H₇, C(═O)—CH(CH₃)₂, butylcarbonyl and C(═O)—CH₂Cl. Particularly preferred aspects of group NRⁱRⁱⁱ are N(di-C₁-C₄-alkyl), in particular N(CH₃)—C₁-C₄-alkyl, such as N(CH₃)₂, N(CH₃)CH₂CH₃, N(CH₃)C₃H₇ and N(CH₃)CH(CH₃)₂.

Further particularly preferred aspects of NRⁱRⁱⁱ are NH-aryl, where aryl is preferably phenyl which is substituted—in particular in the 2- and 6-position—by one to three identical or different groups selected from the group consisting of halogen, CH₃, halo-C₁-C₂-alkyl, halo-C₁-C₂-alkoxy and carboxyl, such as 2-Cl,6-COOH—C₆H₃, 2,6-Cl₂-C₆H₃, 2,6-F₂—C₆H₃, 2,6-Cl₂ 3-C₆H₂, 2-CF₃, 6-CH₂CHF₂—C₆H₃, 2-CF₃,6-OCF₃—C₆H₃ and 2-CF₃, 6-CH₂CHF₂—C₆H₃.

For the compounds of the formula I, the groups R^{c} are preferably selected from the group consisting of halogen, oxo (═O), ═N—R^{d}, C₁-C₄-alkyl, C₁-C₄-haloalkyl, C₂-C₆-alkenyl, C₂-C₆-alkynyl, Z—C₁-C₄-haloalkoxy, C₁-C₄-alkoxy-C₁-C₄-alkyl, C₁-C₄-alkylthio-C₁-C₄-alkyl, C₁-C₄-alkoxy-C₁-C₄-alkoxy-C₁-C₄-alkyl, C₁-C₄-alkylthio-C₁-C₄-alkylthio-C₁-C₄-alkyl, Z—C(═O)—R^{d} and S(O)ₙR^{b}, where R^{b} is preferably C₁-C₄-alkyl or C₁-C₄-haloalkyl and n is 0, 1 or 2.

Particularly preferably, R^{c} is a group selected from the group consisting of halogen, C₁-C₄-alkyl, C₁-C₄-haloalkyl, C₁-C₄-alkoxy, C₁-C₄-alkylthio, C₁-C₄-alkoxy-C₁-C₄-alkyl, C₁-C₄-alkylthio-C₁-C₄-alkyl, C₁-C₄-alkoxy-C₁-C₄-alkoxy-C₁-C₄-alkyl, C₁-C₄-haloalkoxy, C₁-C₄-haloalkylthio, C₃-C₄-alkenyl, C₃-C₄-alkynyl and ═N—C₁-C₄-alkoxy.

Two groups R^{c} together may form a ring which preferably has three to seven ring members and, in addition to carbon atoms, may also contain heteroatoms from the group consisting of O, N and S and which may be unsubstituted or substituted by further groups R^{c}. These substituents R^{c} are preferably selected from the group consisting of halogen, C₁-C₄-alkyl, C₁-C₄-alkoxy and C₁-C₄-haloalkyl.

Groups R^{d} preferred for the compounds of the formula I are selected from the group consisting of OH, C₁-C₈-alkyl, C₁-C₄-haloalkyl, C₃-C₈-alkenyl, C₃-C₈-alkynyl, Z—C₁-C₆-alkoxy, Z—C₁-C₄-haloalkoxy, Z—C₃-C₈-alkenyloxy, Z—C₃-C₈-alkynyloxy and NRⁱRⁱⁱ.

Groups R^{c} and R^{d} are selected independently of one another if a plurality of such groups is present.

In a preferred embodiment of the compounds of the formula I, R¹ is cyano, halogen, nitro, C₁-C₆-alkyl, C₃-C₆-cycloalkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkythio, Z—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{b}, Z-phenoxy or Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, cyclic groups being unsubstituted or partially or fully substituted by R^{c}.

In a more preferred embodiment of the compounds of the formula I, R¹ is cyano, halogen, nitro, C₁-C₆-alkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkythio, Z—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{b}, Z-phenoxy or Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, cyclic groups being unsubstituted or partially or fully substituted by R^{c}.

In a particularly preferred embodiment of the compounds of the formula I, R¹ is halogen, CN, NO₂, C₁-C₄-alkyl, C₃-C₆-cycloalkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkylthio-C₁-C₄-alkyl, C₁-C₄-alkylthio-C₁-C₄-alkylthio-C₁-C₄-alkyl, C₁-C₄-alkoxy, C₁-C₄-haloalkoxy, C₃-C₄-alkenyloxy, C₃-C₄-alkynyloxy, C₁-C₄-alkoxy-C₁-C₄-alkoxy, C₁-C₄-haloalkoxy-C₁-C₄-alkoxy, S(O)ₙ—C₁-C₄-alkyl or S(O)ₙ—C₁-C₄-haloalkyl. Particularly preferably, R¹ is selected from the group consisting of F, Cl, Br, NO₂, CH₃, CH(CH₃)₂, cyclopropyl, CHF₂, CF₃, OCH₃, OCF₃, SCF₃, SO₂CH₃, OCH₂CH₂OCH₃, CH₂OCH₂CH₂OCH₃ and CH₂OCH₂CF₃. Particularly preferably, R¹ is selected from the group consisting of F, Cl, Br, NO₂, CH₃, CH(CH₃)₂, cyclopropyl, CF₃, OCH₃, OCF₃, SCF₃, SO₂CH₃, OCH₂CH₂OCH₃, CH₂OCH₂CH₂OCH₃ and CH₂OCH₂CF₃.

In an especially preferred embodiment of the compounds of the formula I, R¹ is halogen, CN, NO₂, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkylthio-C₁-C₄-alkyl, C₁-C₄-alkylthio-C₁-C₄-alkylthio-C₁-C₄-alkyl, C₁-C₄-alkoxy, C₁-C₄-haloalkoxy, C₃-C₄-alkenyloxy, C₃-C₄-alkynyloxy, C₁-C₄-alkoxy-C₁-C₄-alkoxy, C₁-C₄-haloalkoxy-C₁-C₄-alkoxy, S(O)ₙ—C₁-C₄-alkyl or S(O)ₙ—C₁-C₄-haloalkyl. Particularly preferably, R¹ is selected from the group consisting of F, Cl, Br, NO₂, CH₃, CHF₂, CF₃, OCH₃, OCF₃, SCF₃, SO₂CH₃, OCH₂CH₂OCH₃, CH₂OCH₂CH₂OCH₃ and CH₂OCH₂CF₃. Particularly preferably, R¹ is selected from the group consisting of F, Cl, Br, NO₂, CH₃, CF₃, OCH₃, OCF₃, SCF₃, SO₂CH₃, OCH₂CH₂OCH₃, CH₂OCH₂CH₂OCH₃ and CH₂OCH₂CF₃.

In a further preferred embodiment of the compounds of the formula I, A is C—R². These compounds correspond to the formula I.1

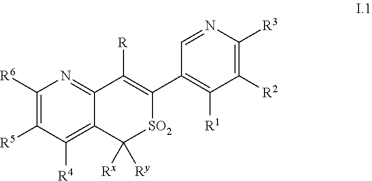

where the variables have the meanings defined at the outset and preferably the meanings mentioned as preferred.

Preferably, in the compounds of the formula I.1, the group
R¹ is halogen, C₁-C₄-alkyl, C₃-C₆-cycloalkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl, in particular F, Cl, Br, I, NO₂, CH₃, CH(CH₃)₂, cyclopropyl, CF₃, OCH₃, OCF₃, OCHF₂, SCF₃, SCHF₂, SO₂CH₃, CH₂OCH₂CH₂OCH₃ or OCH₂CH₂OCH₃; and/or
R³ is H, halogen, CN, NO₂, C₁-C₄-alkyl, C₁-C₄-haloalkyl, C₁-C₄-alkoxy, C₁-C₄-alkylthio, or C₁-C₄-alkylsulfonyl, in particular H, F, Cl, Br, CN, NO₂, CH₃, CH₂CH₃, CF₃, CHF₂, OCH₃, OCF₃, OCHF₂, SCH₃, SO₂CH₃ or SO₂CH₂CH₃.

More preferably, in the compounds of the formula I.1, the group
R¹ is halogen, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl, preferably F, Cl, Br, I, NO₂, CH₃, CF₃, OCH₃, OCF₃, OCHF₂, SCF₃, SCHF₂, SO₂CH₃ or CH₂OCH₂CH₂OCH₃ and in particular F, Cl, Br, NO₂, CH₃, CF₃, OCH₃, OCF₃, OCHF₂, SCF₃, SCHF₂, SO₂CH₃, or CH₂OCH₂CH₂OCH₃; and/or
R³ is H, halogen, CN, NO₂, C₁-C₄-alkyl, C₁-C₄-haloalkyl, C₁-C₄-alkoxy, C₁-C₄-alkylthio or C₁-C₄-alkylsulfonyl, in particular H, F, Cl, Br, CN, NO₂, CH₃, CH₂CH₃, CF₃, CHF₂, OCH₃, OCF₃, OCHF₂, SCH₃, SO₂CH₃ or SO₂CH₂CH₃.

In a preferred embodiment of the compounds of the formula I.1, R² is Z-heterocyclyl where heterocyclyl is a 5- or 6-membered monocyclic or 9- or 10-membered bicyclic, saturated, partially unsaturated or aromatic heterocycle which contains 1, 2, 3 or 4 heteroatoms selected from the group consisting of O, N and S, cyclic groups being unsubstituted or partially or fully substituted by R^{b}.

R² is in this case preferably a 3- to 7-membered monocyclic or 9- or 10-membered bicyclic saturated, partially unsaturated or aromatic heterocycle which is attached via Z¹ and contains 1, 2, 3 or 4 heteroatoms selected from the group consisting of O, N and S, where the cyclic groups are unsubstituted or partially or fully substituted by groups R^{c}.

In a further preferred aspect of the compounds of the formula I.1, R² is a 3- to 7-membered monocyclic or 9- or 10-membered bicyclic saturated, partially unsaturated or aromatic heterocycle which is attached directly or via C₁-C₄-alkyleneoxy, C₁-C₄-oxyalkylene or C₁-C₄-alkyleneoxy-C₁-C₄-alkylene, which contains 1, 2, 3 or 4 heteroatoms selected from the group consisting of O, N and S and which may be substituted as defined at the outset.

A preferred aspect of group R² relates to five- or six-membered saturated or partially unsatured heterocycles, such as, for example, isoxazoline, tetrazolone, 1,2-dihydrotetrazolone, 1,4-dihydrotetrazolone, tetrahydrofuran, dioxolane, piperidine, morpholine and piperazine. Particular preference is given to 3-isoxazoline, 5-isoxazoline, 1-tetrazolone, 2-tetrazolone, [1,3]dioxolane-2 and N-morpholine. Especially preferred are: 4,5-dihydroisoxazole-3, unsubstituted or substituted by 5-CH₃, 5-CH₂F or 5-CHF₂; 4,5-dihydroisoxazole-5, unsubstituted or substituted by 3-CH₃, 3-OCH₃, 3-CH₂OCH₃, 3-CH₂SCH₃; 1-methyl-5-oxo-1,5-dihydrotetrazole-2; 4-methyl-5-oxo-4,5-dihydrotetrazole-1 and N-morpholine.

A further preferred aspect of group R² relates to five- or six-membered aromatic heterocycles, such as, for example, isoxazole, pyrazole, thiazole, furyl, pyridine, pyrimidine and pyrazine. Particular preference is given to 3-isoxazole, 5-isoxazole, 3-pyrazole, 5-pyrazole, 2-thiazole, 2-oxazole, 2-furyl. Especially preferred are: 3-isoxazole, 5-methyl-3-isoxazole, 5-isoxazole, 3-methyl-5-isoxazole, 1-methyl-1H-pyrazole-3,2-methyl-2H-pyrazole-3 and thiazole-2.

In a preferred aspect of the compounds of the formula I, the groups R^{c} independently of one another are Z—CN, Z—OH, Z—NO₂, Z-halogen, oxo (═O), ═N—R^{d}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{d}, NRⁱRⁱⁱ, Z-(tri-C₁-C₄-alkyl)silyl, Z-phenyl or S(O)ₙR^{b}.

In a preferred aspect of heterocyclic groups R², the groups R^{c} independently of one another are preferably C₁-C₄-alkyl, C₁-C₄-haloalkyl, C₁-C₄-alkoxy, C₁-C₄-alkoxy-C₁-C₄-alkyl, C₁-C₄-alkylthio-C₁-C₄-alkyl, C₁-C₄-alkylthio or C₁-C₄-alkylsulfonyl. Especially preferred are CH₃, C₂H₅, CH₂F, CF₂H, CF₃, OCH₃, CH₂OCH₃, CH₂SCH₃, SCH₃ and SO₂CH₃.

The group R^{b} is preferably C₁-C₈-alkyl.

In a preferred aspect, the group Z¹ is a covalent bond.

In a further preferred aspect, the group Z¹ is C₁-C₄-alkyleneoxy, in particular OCH₂ or OCH₂CH₂.

In a further preferred aspect, the group Z¹ is C₁-C₄-oxyalkylene, in particular CH₂O or CH₂CH₂O.

In a further preferred aspect, the group Z¹ is C₁-C₄-alkyleneoxy-C₁-C₄-alkylene, in particular OCH₂OCH₂ or OCH₂CH₂OCH₂.

Particularly preferred aspects of heterocycles attached via Z¹ include tetrahydrofuran-2-ylmethoxymethyl and [1,3]dioxolan-2-ylmethoxy.

In a further preferred embodiment of the compounds of the formula I.1, R² is phenyl which is attached via Z¹ or oxygen and is unsubstituted or substituted by C₁-C₄-alkyl, C₁-C₄-alkoxy, C₁-C₄-haloalkyl, C₁-C₄-alkoxy-C₁-C₄-alkyl or C₁-C₄-alkoxy-C₁-C₄-alkoxy.

Particular preference is given in this case to a phenyl group which may be partially or fully substituted—preferably mono-, di- or trisubstituted, in particular monosubstituted—by groups R^{c}. Groups R^{c} preferred for this aspect include: C₁-C₂-alkyl, C₁-C₄-alkoxy, C₁-C₂-haloalkyl, C₁-C₂-alkoxy-C₁-C₂-alkyl or C₁-C₂-alkoxy-C₁-C₂-alkoxy. Particular preference is given to CH₃, C₂H₅, OCH₃, OC₂H₅, CHF₂, CF₃, OCHF₂, OCF₃, OCH₂OCH₃ and OCH₂CH₂OCH₃. Special preference is given to C₁-C₄-alkoxy, such as OCH₃ or OC₂H₅. A group R^{c} is preferably in position 4. A particularly preferred phenyl group R² is a group P:

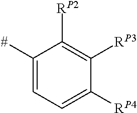

wherein # denotes the bond via which the group R² is attached and the substituents are selected from R^{c} and are in particular:

R^{P2} H or F;

R^{P3} H, F, Cl or OCH₃; and

R^{P4} H, F, Cl, CH₃, CF₃, OCH₃, OCH₂OCH₃ or OCH₂CH₂OCH₃.

In another preferred embodiment of the compounds of the formula I.1, R² is an aliphatic group selected from the group consisting of C₁-C₈-alkyl, C₂-C₆-haloalkyl, C₁-C₄-alkoxy-C₁-C₄-alkyl, C₂-C₈-alkenyl, C₂-C₈-alkynyl, C₂-C₈-haloalkenyl, C₂-C₈-haloalkynyl, C₁-C₆-alkoxy, Z—C₁-C₄-alkoxy-C₁-C₄-alkoxy, Z—C₁-C₄-haloalkoxy-C₁-C₄-alkoxy, Z—C₁-C₆-haloalkoxy, C₂-C₈-alkenyloxy, C₂-C₈-alkynyloxy, Z—C₁-C₄-alkythio, Z—C₁-C₆-haloalkylthio, Z—C(═O)—R^{d} or S(O)ₙR^{b}.

In yet another preferred embodiment of the compounds of the formula I.1, R² is aliphatic group selected from the group consisting of C₁-C₆-alkyl, C₁-C₄-alkoxy-C₁-C₄-alkyl, C₁-C₄-haloalkoxy-C₁-C₄-alkyl, C₂-C₆-alkenyl, C₂-C₆-alkynyl, C₂-C₄-alkoxy, C₂-C₄-haloalkoxy, C₃-C₆-alkenyloxy, C₃-C₆-alkynyloxy, C₃-C₆-haloalkenyloxy, C₃-C₆-haloalkynyloxy, C₁-C₄-alkoxycarbonyl, S(O)₂—C₁-C₈-alkyl, S(O)₂—C₁-C₈-haloalkyl and N—(C₁-C₄-alkyl)amino-N-sulfonyl-C₁-C₄-alkyl.

In a particularly preferred aspect of these compounds of the formula I.1, R² is an aliphatic group selected from the group consisting of C₁-C₆-alkyl, C₁-C₄-alkoxy-C₁-C₄-alkyl, C₁-C₄-haloalkoxy-C₁-C₄-alkyl, C₂-C₆-alkenyl, C₂-C₆-alkynyl, C₂-C₄-alkoxy, C₁-C₄-haloalkoxy, C₃-C₆-alkenyloxy, C₃-C₆-alkynyloxy, C₃-C₆-haloalkenyloxy, C₃-C₆-haloalkynyloxy, C₁-C₄-alkoxycarbonyl, S(O)₂—C₁-C₄-alkyl and S(O)₂—C₁-C₆-haloalkyl.

Particularly preferred aliphatic groups R² include C₂-C₄-alkenyl, C₂-C₄-alkynyl, C₁-C₂-haloalkoxy-C₁-C₂-alkyl, C₃-C₄-alkenyloxy, C₃-C₄-alkynyloxy, C₁-C₄-alkylsulfonyl, C₁-C₄-alkoxy, C₁-C₄-alkoxycarbonyl and S(O)₂—C₁-C₄-alkyl. Special preference is given to CH═CH₂, CH═CHCH₃, CH₂OCH₂CF₃, OC₂H₅, OCH₂CH═CH₂, OCH₂C≡CH, OCH₂CH₂OCH₃, COOCH₃, COOC₂H₅ and SO₂CH₃, SO₂C₂H₅ and SO₂CH(CH₃)₂.

In a further preferred aspect, R² together with the group attached to the adjacent carbon atom forms a five- to ten-membered saturated, partially unsaturated or fully unsaturated mono- or bicyclic ring which, in addition to carbon atoms, may contain 1, 2 or 3 heteroatoms selected from the group consisting of O, N and S and which may be substituted by further groups R^{c}. In a particularly preferred aspect, R² together with R¹ or R³ forms a five- to ten-membered mono- or bicyclic, saturated, partially unsaturated or fully unsaturated ring which contains 1, 2, 3 or 4 heteroatoms selected from the group consisting of O, N and S and which may be partially or fully substituted by groups R^{c}. Suitable are, for example, the following: 4-dihydro-2H-thiopyrano[2,3-b]pyridine 1,1-dioxide, 3,4-dihydro-2H-thiopyrano[3,2-b]pyridine 1,1-dioxide, 2,3-dihydro-[1,4]dithiino[2,3-b]pyridine 1,1,4,4-tetraoxide, 1H-thiazolo[5,4-b]pyridin-2-one, 2,3-dihydrothieno[2,3-b]pyridine 1,1-dioxide, 1,8-naphthyridine, 1,5-naphthyridine, 1,7-naphthyridine and isothiazolo[5,4-b]pyridine.

Preferably, R² together with R¹ or R³ forms a five- or six-membered monocyclic, saturated, partially unsaturated or fully unsaturated ring.

In a further preferred embodiment of the compounds of the formula I (in particular of the formula I.1), R³ is hydrogen, cyano, halogen, nitro, C₁-C₄-alkyl, C₁-C₄-haloalkyl, C₁-C₄-alkoxy, C₁-C₄-haloalkoxy, C₂-C₄-alkenyl, C₂-C₄-alkynyl, C₂-C₄-alkenyloxy, C₂-C₄-alkynyloxy or S(O)ₙR^{b}. In a particularly preferred embodiment of the compounds of the formula I (in particular of the formula I.1), R³ is hydrogen, halogen, CN, NO₂, C₁-C₄-alkyl, C₁-C₄-haloalkyl, C₁-C₄-alkoxy, C₁-C₄-haloalkoxy, S(O)ₙ—C₁-C₄-alkyl and S(O)ₙ—C₁-C₄-haloalkyl, where n is preferably 0 or 2. Particularly preferably, R³ is selected from the group consisting of H, F, Cl, Br, CN, NO₂, CH₃, CF₃, CHF₂, OCH₃, OCF₃, OCHF₂, SCH₃, SCF₃, SCHF₂, SO₂CH₃ and SO₂CH₂CH₃.

In further preferred aspects of the formula I.1, the groups R¹, R² and R³ together form a substitution pattern selected from the group consisting of:

R¹═Cl, R²═H, R³═Cl;

R¹═Cl, R²═H, R³═CF₃;

R¹═Cl, R²═H, R³═SO₂CH₃;

R¹═Cl, R²═H, R³═OCH₃;

R¹═Cl, R²═H, R³═CH₃;

R¹═CH₃, R²═H, R³═Cl;

R¹═CH₃, R²═H, R³═CF₃;

R¹═CH₃, R²═H, R³═SO₂CH₃;

R¹═CH₃, R²═H, R³═OCH₃;

R¹═CF₃, R²═H, R³═CH₃;

R¹═CF₃, R²═H, R³═Cl;

R¹═CF₃, R²═H, R³═CF₃;

R¹═CF₃, R²═H, R³═SO₂CH₃;

R¹═CF₃, R²═H, R³═OCH₃;

R¹═SO₂CH₃, R²═H, R³═CH₃,

R¹═SO₂CH₃, R²═H, R³═Cl;

R¹═SO₂CH₃, R²═H, R³═CF₃;

R¹═SO₂CH₃, R²═H, R³═SO₂CH₃;

R¹═SO₂CH₃, R²═H, R³═OCH₃; and

R¹═SO₂CH₃, R²═H, R³═CH₃.

In a further preferred embodiment of the compounds of the formula I, A is N. These compounds correspond to formula I.2

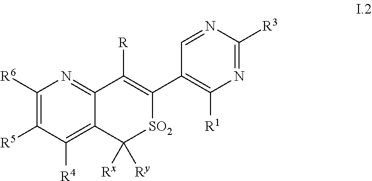

in which the variables have the meanings defined at the outset and preferably those mentioned above. In one embodiment, R¹ and R³ are not halogen.

Especially preferably, in compounds of the formula I.2 the group
R¹ is halogen, nitro, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl, in particular NO₂, CH₃, CF₃, CH₂OCH₂CH₂OCH₃, OCH₃, OCF₃, OCHF₂, SCF₃, SCHF₂ or SO₂CH₃;
R³ is H, halogen, CN, NO₂, C₁-C₄-alkyl, O—C₃-C₆-cycloalkyl, C₁-C₄-haloalkyl, C₁-C₄-alkoxy, C₁-C₄-alkylthio, C₁-C₄-alkylsulfonyl, NRⁱRⁱⁱ wherein Rⁱ and Rⁱⁱ are, independently from one another, hydrogen or Z—C(═O)—R^{d} wherein Z is a covalent bond and R^{d} is C₁-C₈-alkyl, or Z—NRⁱSO₂Rⁱⁱ wherein Z is a covalent bond, Rⁱ is hydrogen or C₁-C₄-alkyl and Rⁱⁱ is C₁-C₄-alkyl, in particular H, CN, NO₂, CH₃, CH₂CH₃, O-cyclobutyl, CF₃, CHF₂, OCH₃, OCH(CH₃)₂, OCF₃, OCHF₂, SCH₃, SO₂CH₃, SO₂CH₂CH₃. NH₂, NHCOCH₂CH(CH₃)₂, NHSO₂CH₃ or N(CH₃)SO₂CH₃.

Especially preferably, in compounds of the formula I.2 the group
R¹ is halogen, nitro, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl, in particular NO₂, CH₃, CF₃, CH₂OCH₂CH₂OCH₃, OCH₃, OCF₃, OCHF₂, SCF₃, SCHF₂ or SO₂CH₃;
R³ is H, halogen, CN, NO₂, C₁-C₄-alkyl, C₁-C₄-haloalkyl, C₁-C₄-alkoxy, C₁-C₄-alkylthio or C₁-C₄-alkylsulfonyl, in particular H, CN, NO₂, CH₃, CH₂CH₃, CF₃, CHF₂, OCH₃, OCF₃, OCHF₂, SCH₃, SO₂CH₃ or SO₂CH₂CH₃.

In another preferred embodiment of the compounds of the formula I, R⁴ is hydrogen, C₁-C₄-alkyl, C₁-C₄-haloalkyl, C₁-C₄-alkoxy, C₁-C₄-alkylthio, C₁-C₄-haloalkoxy or C₁-C₄-haloalkylthio, particularly preferably H, CH₃, CF₃, CHF₂, OCH₃, OCF₃, OCHF₂, SCH₃, SCF₃ or SCHF₂, in particular H.

In further preferred embodiments:

R⁵ is H, OH, CN, halogen, C₁-C₄-alkyl, C₁-C₄-alkoxy or C₁-C₄-haloalkyl, preferably H or halogen, especially H or F and in particular H; and

R⁶ is H, OH, CN, halogen, C₁-C₄-alkyl, C₁-C₄-alkoxy or C₁-C₄-haloalkyl, in particular H.

In another preferred embodiment of the compounds of the formula I, at least one of the groups R⁵ and R⁶ is hydrogen.

In another preferred embodiment of the compounds of the formula I, at least one of the groups R⁴ and R⁶ is hydrogen.

In another preferred embodiment of the compounds of the formula I, R⁵ and R⁶ are both hydrogen.

In another preferred embodiment of the compounds of the formula I, R⁴ and R⁶ are both hydrogen.

In another preferred embodiment of the compounds of the formula I, R⁴ and R⁶ are both hydrogen and R⁵ is halogen, in particular fluorine.

R^{x} and R^{y}, independently of one another, are preferably H, C₁-C₅-alkyl, such as CH₃, C₂H₅, n-C₃H₇, CH(CH₃)₂, n-C₃H₉, or C(CH₃)₃; C₃-C₅-alkenyl, C₃-C₅-alkynyl, such as CH₂CH═CH₂, CH₂C(CH₃)═CH₂, CH₂CH₂H═CH₂, CH₂CH₂C(CH₃)—CH₂, CH₂CH₂CH₂CH═CH₂, CH₂C≡CH, or C₁-C₅-haloalkyl, such as CH₂CF₃, CH₂CHF₂. More preferably, R^{x} and R^{y} are, independently of one another, H, C₁-C₅-alkyl or C₁-C₄-haloalkyl. Particularly preferably, R^{x} and R^{y}, independently of one another, are H or C₁-C₅-alkyl.

In another preferred embodiment of the compounds of the formula I, R⁴, R⁵ and R⁶ are hydrogen. These compounds correspond to formula I.3

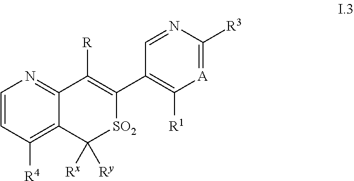

in which the variables have the meanings defined at the outset and preferably those mentioned above.

Preferably, in compounds of the formula I.3 the group
R is hydroxy or O—R^{A} wherein R^{A} is C₁-C₆-alkylcarbonyl, preferably hydroxy, prop-2-ylcarbonyloxy, cyclopropylcarbonyloxy or 2-methyl-prop-2-ylcarbonyloxy and more preferably hydroxy or 2-methyl-prop-2-ylcarbonyloxy;
R¹ is halogen, C₁-C₄-alkyl, C₃-C₆-cyclopropyl, C₁-C₄-haloalkyl, C₁-C₄-alkoxy, C₁-C₄-alkoxy-C₁-C₄-alkoxy, or —SO₂—C₁-C₄-alkyl, in particular chloro, methyl, cyclopropyl, isopropyl, trifluoromethyl, methoxy, OCH₂CH₂—OCH₃ or methylsulfonyl;
A is N or C—R²;
R² is hydrogen, chlorine, N(CH₃)SO₂CH₃ or methylsulfonyl, preferably hydrogen or N(CH₃)SO₂CH₃ and in particular hydrogen;
R³ is halogen, C₁-C₄-alkyl, C₁-C₄-alkoxy, —O—C₃-C₆-cycloalkyl, C₁-C₄-haloalkyl, C₁-C₄-alkylthio, NRⁱRⁱⁱ wherein Rⁱ and Rⁱⁱ are, independently from one another, hydrogen or Z—C(═O)—R^{d} wherein Z is a covalent bond and R^{d} is C₁-C₈-alkyl, Z—NRⁱSO₂Rⁱⁱ wherein Z is a covalent bond, Rⁱ is hydrogen or C₁-C₄-alkyl and Rⁱⁱ is C₁-C₄-alkyl, or —SO₂—C₁-C₄-alkyl (in particular halogen, C₁-C₄-alkyl, C₁-C₄-alkoxy, —O—C₃-C₆-cycloalkyl, C₁-C₄-haloalkyl, C₁-C₄-alkylthio, NRⁱRⁱⁱ wherein Rⁱ and Rⁱⁱ are, independently from one another, hydrogen or Z—C(═O)—R^{d} wherein Z is a covalent bond and R^{d} is C₁-C₈-alkyl, or Z—NRⁱSO₂Rⁱⁱ wherein Z is a covalent bond, Rⁱ is hydrogen or C₁-C₄-alkyl and Rⁱⁱ is C₁-C₄-alkyl), preferably chloro, bromo, methyl, isopropyl, methoxy, isopropoxy, O-cyclobutyl, trifluoromethyl, methylthio, —NH₂, NHCOCH₂CH(CH₃)₂, NHSO₂CH₃, N(CH₃)SO₂CH₃ or methylsulfonyl (in particular chloro, bromo, methyl, isopropyl, methoxy, isopropoxy, O-cyclobutyl, trifluoromethyl, methylthio, —NH₂, NHCOCH₂CH(CH₃)₂, NHSO₂CH₃ or N(CH₃)SO₂CH₃);
R^{x} is H, C₁-C₅-alkyl or C₁-C₄-haloalkyl, preferably hydrogen or C₁-C₅-alkyl and in particular hydrogen or methyl; and
R^{y} is hydrogen, C₁-C₅-alkyl or C₁-C₄-haloalkyl, preferably hydrogen or C₁-C₅-alkyl and in particular hydrogen or methyl.

Especially preferably, in compounds of the formula I.3 the group
R is hydroxy or O—R^{A} wherein R^{A} is C₁-C₆-alkylcarbonyl, in particular hydroxy, prop-2-ylcarbonyloxy, cyclopropylcarbonyloxy or 2-methyl-prop-2-ylcarbonyloxy;
R¹ is halogen, C₁-C₄-alkyl, C₁-C₄-haloalkyl or —SO₂—C₁-C₄-alkyl, in particular chloro, methyl or trifluoromethyl;
A is N or C—R²;
R² is hydrogen, chlorine or methylsulfonyl, in particular hydrogen;
R³ is halogen, C₁-C₄-alkyl, C₁-C₄-alkoxy, C₁-C₄-haloalkyl or —SO₂—C₁-C₄-alkyl, in particular chloro, methoxy, trifluoromethyl or methylsulfonyl;
R^{x} is H, C₁-C₅-alkyl or C₁-C₄-haloalkyl, preferably hydrogen or C₁-C₅-alkyl and in particular hydrogen or methyl; and
R^{y} is hydrogen, C₁-C₅-alkyl or C₁-C₄-haloalkyl, preferably hydrogen or C₁-C₅-alkyl and in particular hydrogen or methyl.

A further embodiment relates to the N-oxides of the compounds of the formula I.

A further embodiment relates to salts of the compounds of the formula I, in particular those which are obtainable by quaternization of a pyridine nitrogen atom, which may preferably take place by alkylation or arylation of the compounds of the formula I. Preferred salts of the compounds are thus the N-alkyl salts, in particular the N-methyl salts, and the N-phenyl salts.

In particular with a view to their use, preference is given to the compounds of the formula I compiled in the tables below, which compounds correspond to the formula I.a. The groups mentioned for a substituent in the tables are furthermore per se, independently of the combination in which they are mentioned, a particularly preferred aspect of the substituent in question.
Table 1

Compounds of the formula I, in which R^{x} is H, R^{y} is H, R is OH and the combination of R¹ to R³ for a compound corresponds in each case to one row of table A
Table 2

Compounds of the formula I, in which R^{x} is H, R^{y} is CH₃, R is OH and the combination of R¹ to R³ for a compound corresponds in each case to one row of table A
Table 3

Compounds of the formula I, in which R^{x} is CH₃, R^{y} is CH₃, R is OH and the combination of R¹ to R³ for a compound corresponds in each case to one row of table A
Table 4

Compounds of the formula I, in which R^{x} is H, R^{y} is CH₂CH₃, R is OH and the combination of R¹ to R³ for a compound corresponds in each case to one row of table A

TABLE A
Compounds of the formula I which correspond to the formula I.a
I.a

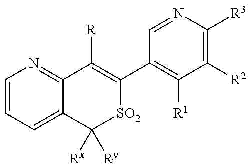

| No. | R¹ | R² | R³ |
|---|---|---|---|
| I.a.1 | Cl | H | H |
| I.a.2 | Br | H | H |
| I.a.3 | I | H | H |
| I.a.4 | CH₃ | H | H |
| I.a.5 | CF₃ | H | H |
| I.a.6 | SO₂CH₃ | H | H |
| I.a.7 | Cl | Cl | H |
| I.a.8 | Br | Cl | H |
| I.a.9 | I | Cl | H |
| I.a.10 | CH₃ | Cl | H |
| I.a.11 | CF₃ | Cl | H |
| I.a.12 | SO₂CH₃ | Cl | H |
| I.a.13 | Cl | 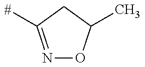 | H |
| I.a.14 | Br | 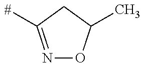 | H |
| I.a.15 | I | 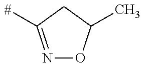 | H |
| I.a.16 | CH₃ | 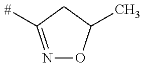 | H |
| I.a.17 | CF₃ | 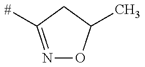 | H |
| I.a.18 | SO₂CH₃ | 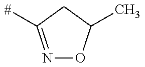 | H |
| I.a.19 | Cl | 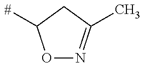 | H |
| I.a.20 | Br | 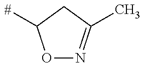 | H |
| I.a.21 | I | 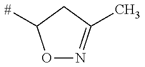 | H |
| I.a.22 | CH₃ | 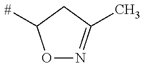 | H |
| I.a.23 | CF₃ | 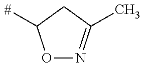 | H |
| I.a.24 | SO₂CH₃ | 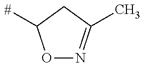 | H |
| I.a.25 | Cl | N(CH₃)SO₂CH₃ | H |
| I.a.26 | Br | N(CH₃)SO₂CH₃ | H |
| I.a.27 | I | N(CH₃)SO₂CH₃ | H |
| I.a.28 | CH₃ | N(CH₃)SO₂CH₃ | H |
| I.a.29 | CF₃ | N(CH₃)SO₂CH₃ | H |
| I.a.30 | SO₂CH₃ | N(CH₃)SO₂CH₃ | H |
| I.a.31 | Cl | CH₂OCH₂CH₂OCH₃ | H |
| I.a.32 | Br | CH₂OCH₂CH₂OCH₃ | H |
| I.a.33 | I | CH₂OCH₂CH₂OCH₃ | H |
| I.a.34 | CH₃ | CH₂OCH₂CH₂OCH₃ | H |
| I.a.35 | CF₃ | CH₂OCH₂CH₂OCH₃ | H |
| I.a.36 | SO₂CH₃ | CH₂OCH₂CH₂OCH₃ | H |
| I.a.37 | Cl | CH₂OCH₂CF₃ | H |
| I.a.38 | Br | CH₂OCH₂CF₃ | H |
| I.a.39 | I | CH₂OCH₂CF₃ | H |
| I.a.40 | CH₃ | CH₂OCH₂CF₃ | H |
| I.a.41 | CF₃ | CH₂OCH₂CF₃ | H |
| I.a.42 | SO₂CH₃ | CH₂OCH₂CF₃ | H |
| I.a.43 | Cl | 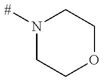 | H |
| I.a.44 | Br | 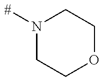 | H |
| I.a.45 | I | 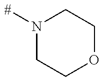 | H |
| I.a.46 | CH₃ | 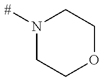 | H |
| I.a.47 | CF₃ | 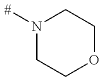 | H |
| I.a.48 | SO₂CH₃ | 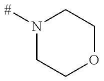 | H |
| I.a.49 | Cl | SO₂CH₃ | H |
| I.a.50 | Br | SO₂CH₃ | H |
| I.a.51 | I | SO₂CH₃ | H |
| I.a.52 | CH₃ | SO₂CH₃ | H |
| I.a.53 | CF₃ | SO₂CH₃ | H |
| I.a.54 | SO₂CH₃ | SO₂CH₃ | H |
| I.a.55 | Cl | H | F |
| I.a.56 | Br | H | F |
| I.a.57 | I | H | F |
| I.a.58 | CH₃ | H | F |
| I.a.59 | CF₃ | H | F |
| I.a.60 | SO₂CH₃ | H | F |
| I.a.61 | Cl | Cl | F |
| I.a.62 | Br | Cl | F |
| I.a.63 | I | Cl | F |
| I.a.64 | CH₃ | Cl | F |
| I.a.65 | CF₃ | Cl | F |
| I.a.66 | SO₂CH₃ | Cl | F |
| I.a.67 | Cl | 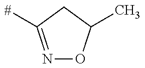 | F |
| I.a.68 | Br | 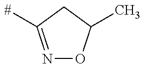 | F |
| I.a.69 | I | 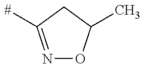 | F |
| I.a.70 | CH₃ | 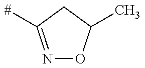 | F |
| I.a.71 | CF₃ | 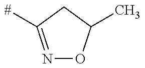 | F |
| I.a.72 | SO₂CH₃ | 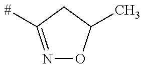 | F |
| I.a.73 | Cl | 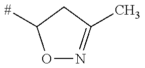 | F |
| I.a.74 | Br | 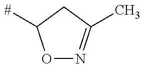 | F |
| I.a.75 | I | 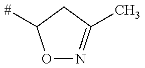 | F |
| I.a.76 | CH₃ | 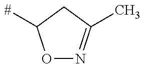 | F |
| I.a.77 | CF₃ | 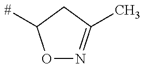 | F |
| I.a.78 | SO₂CH₃ | 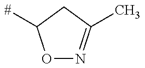 | F |
| I.a.79 | Cl | N(CH₃)SO₂CH₃ | F |
| I.a.80 | Br | N(CH₃)SO₂CH₃ | F |
| I.a.81 | I | N(CH₃)SO₂CH₃ | F |
| I.a.82 | CH₃ | N(CH₃)SO₂CH₃ | F |
| I.a.83 | CF₃ | N(CH₃)SO₂CH₃ | F |
| I.a.84 | SO₂CH₃ | N(CH₃)SO₂CH₃ | F |
| I.a.85 | Cl | CH₂OCH₂CH₂OCH₃ | F |
| I.a.86 | Br | CH₂OCH₂CH₂OCH₃ | F |
| I.a.87 | I | CH₂OCH₂CH₂OCH₃ | F |
| I.a.88 | CH₃ | CH₂OCH₂CH₂OCH₃ | F |
| I.a.89 | CF₃ | CH₂OCH₂CH₂OCH₃ | F |
| I.a.90 | SO₂CH₃ | CH₂OCH₂CH₂OCH₃ | F |
| I.a.91 | Cl | CH₂OCH₂CF₃ | F |
| I.a.92 | Br | CH₂OCH₂CF₃ | F |
| I.a.93 | I | CH₂OCH₂CF₃ | F |
| I.a.94 | CH₃ | CH₂OCH₂CF₃ | F |
| I.a.95 | CF₃ | CH₂OCH₂CF₃ | F |
| I.a.96 | SO₂CH₃ | CH₂OCH₂CF₃ | F |
| I.a.97 | Cl | 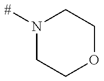 | F |
| I.a.98 | Br | 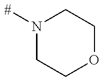 | F |
| I.a.99 | I | 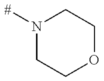 | F |
| I.a.100 | CH₃ | 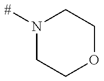 | F |
| I.a.101 | CF₃ | 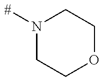 | F |
| I.a.102 | SO₂CH₃ | 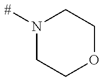 | F |
| I.a.103 | Cl | SO₂CH₃ | F |
| I.a.104 | Br | SO₂CH₃ | F |
| I.a.105 | I | SO₂CH₃ | F |
| I.a.106 | CH₃ | SO₂CH₃ | F |
| I.a.107 | CF₃ | SO₂CH₃ | F |
| I.a.108 | SO₂CH₃ | SO₂CH₃ | F |
| I.a.109 | Cl | H | Cl |
| I.a.110 | Br | H | Cl |
| I.a.111 | I | H | Cl |
| I.a.112 | CH₃ | H | Cl |
| I.a.113 | CF₃ | H | Cl |
| I.a.114 | SO₂CH₃ | H | Cl |
| I.a.115 | Cl | Cl | Cl |
| I.a.116 | Br | Cl | Cl |
| I.a.117 | I | Cl | Cl |
| I.a.118 | CH₃ | Cl | Cl |
| I.a.119 | CF₃ | Cl | Cl |
| I.a.120 | SO₂CH₃ | Cl | Cl |
| I.a.121 | Cl | 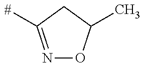 | Cl |
| I.a.122 | Br | 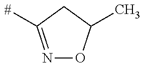 | Cl |
| I.a.123 | I | 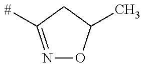 | Cl |
| I.a.124 | CH₃ | 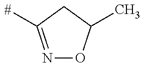 | Cl |
| I.a.125 | CF₃ | 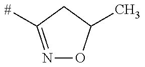 | Cl |
| I.a.126 | SO₂CH₃ | 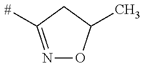 | Cl |
| I.a.127 | Cl | 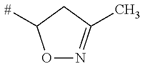 | Cl |
| I.a.128 | Br | 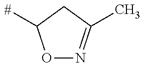 | Cl |
| I.a.129 | I | 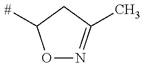 | Cl |
| I.a.130 | CH₃ | 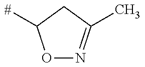 | Cl |
| I.a.131 | CF₃ | 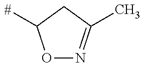 | Cl |
| I.a.132 | SO₂CH₃ | 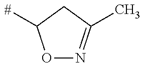 | Cl |
| I.a.133 | Cl | N(CH₃)SO₂CH₃ | Cl |
| I.a.134 | Br | N(CH₃)SO₂CH₃ | Cl |
| I.a.135 | I | N(CH₃)SO₂CH₃ | Cl |
| I.a.136 | CH₃ | N(CH₃)SO₂CH₃ | Cl |
| I.a.137 | CF₃ | N(CH₃)SO₂CH₃ | Cl |
| I.a.138 | SO₂CH₃ | N(CH₃)SO₂CH₃ | Cl |
| I.a.139 | Cl | CH₂OCH₂CH₂OCH₃ | Cl |
| I.a.140 | Br | CH₂OCH₂CH₂OCH₃ | Cl |
| I.a.141 | I | CH₂OCH₂CH₂OCH₃ | Cl |
| I.a.142 | CH₃ | CH₂OCH₂CH₂OCH₃ | Cl |
| I.a.143 | CF₃ | CH₂OCH₂CH₂OCH₃ | Cl |
| I.a.144 | SO₂CH₃ | CH₂OCH₂CH₂OCH₃ | Cl |
| I.a.145 | Cl | CH₂OCH₂CF₃ | Cl |
| I.a.146 | Br | CH₂OCH₂CF₃ | Cl |
| I.a.147 | I | CH₂OCH₂CF₃ | Cl |
| I.a.148 | CH₃ | CH₂OCH₂CF₃ | Cl |
| I.a.149 | CF₃ | CH₂OCH₂CF₃ | Cl |
| I.a.150 | SO₂CH₃ | CH₂OCH₂CF₃ | Cl |
| I.a.151 | Cl | 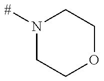 | Cl |
| I.a.152 | Br | 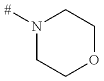 | Cl |
| I.a.153 | I | 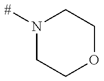 | Cl |
| I.a.154 | CH₃ | 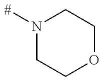 | Cl |
| I.a.155 | CF₃ | 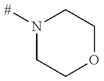 | Cl |
| I.a.156 | SO₂CH₃ | 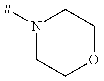 | Cl |
| I.a.157 | Cl | SO₂CH₃ | Cl |
| I.a.158 | Br | SO₂CH₃ | Cl |
| I.a.159 | I | SO₂CH₃ | Cl |
| I.a.160 | CH₃ | SO₂CH₃ | Cl |
| I.a.161 | CF₃ | SO₂CH₃ | Cl |
| I.a.162 | SO₂CH₃ | SO₂CH₃ | Cl |
| I.a.163 | Cl | H | Br |
| I.a.164 | Br | H | Br |
| I.a.165 | I | H | Br |
| I.a.166 | CH₃ | H | Br |
| I.a.167 | CF₃ | H | Br |
| I.a.168 | SO₂CH₃ | H | Br |
| I.a.169 | Cl | Cl | Br |
| I.a.170 | Br | Cl | Br |
| I.a.171 | I | Cl | Br |
| I.a.172 | CH₃ | Cl | Br |
| I.a.173 | CF₃ | Cl | Br |
| I.a.174 | SO₂CH₃ | Cl | Br |
| I.a.175 | Cl | 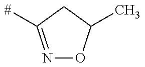 | Br |
| I.a.176 | Br | 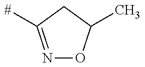 | Br |
| I.a.177 | I | 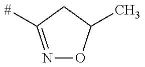 | Br |
| I.a.178 | CH₃ | 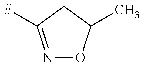 | Br |
| I.a.179 | CF₃ | 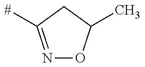 | Br |
| I.a.180 | SO₂CH₃ | 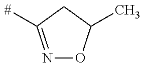 | Br |
| I.a.181 | Cl | 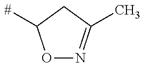 | Br |
| I.a.182 | Br | 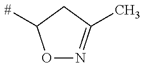 | Br |
| I.a.183 | I | 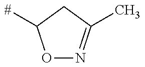 | Br |
| I.a.184 | CH₃ | 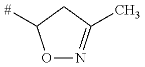 | Br |
| I.a.185 | CF₃ | 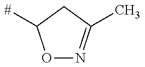 | Br |
| I.a.186 | SO₂CH₃ | 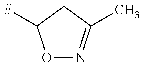 | Br |
| I.a.187 | Cl | N(CH₃)SO₂CH₃ | Br |
| I.a.188 | Br | N(CH₃)SO₂CH₃ | Br |
| I.a.189 | I | N(CH₃)SO₂CH₃ | Br |
| I.a.190 | CH₃ | N(CH₃)SO₂CH₃ | Br |
| I.a.191 | CF₃ | N(CH₃)SO₂CH₃ | Br |
| I.a.192 | SO₂CH₃ | N(CH₃)SO₂CH₃ | Br |
| I.a.193 | Cl | CH₂OCH₂CH₂OCH₃ | Br |
| I.a.194 | Br | CH₂OCH₂CH₂OCH₃ | Br |
| I.a.195 | I | CH₂OCH₂CH₂OCH₃ | Br |
| I.a.196 | CH₃ | CH₂OCH₂CH₂OCH₃ | Br |
| I.a.197 | CF₃ | CH₂OCH₂CH₂OCH₃ | Br |
| I.a.198 | SO₂CH₃ | CH₂OCH₂CH₂OCH₃ | Br |
| I.a.199 | Cl | CH₂OCH₂CF₃ | Br |
| I.a.200 | Br | CH₂OCH₂CF₃ | Br |
| I.a.201 | I | CH₂OCH₂CF₃ | Br |
| I.a.202 | CH₃ | CH₂OCH₂CF₃ | Br |
| I.a.203 | CF₃ | CH₂OCH₂CF₃ | Br |
| I.a.204 | SO₂CH₃ | CH₂OCH₂CF₃ | Br |
| I.a.205 | Cl | 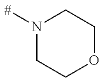 | Br |
| I.a.206 | Br | 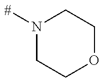 | Br |
| I.a.207 | I | 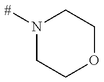 | Br |
| I.a.208 | CH₃ | 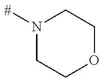 | Br |
| I.a.209 | CF₃ | 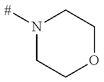 | Br |
| I.a.210 | SO₂CH₃ | 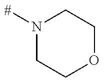 | Br |
| I.a.211 | Cl | SO₂CH₃ | Br |
| I.a.212 | Br | SO₂CH₃ | Br |
| I.a.213 | I | SO₂CH₃ | Br |
| I.a.214 | CH₃ | SO₂CH₃ | Br |
| I.a.215 | CF₃ | SO₂CH₃ | Br |
| I.a.216 | SO₂CH₃ | SO₂CH₃ | Br |
| I.a.217 | Cl | H | I |
| I.a.218 | Br | H | I |
| I.a.219 | I | H | I |
| I.a.220 | CH₃ | H | I |
| I.a.221 | CF₃ | H | I |
| I.a.222 | SO₂CH₃ | H | I |
| I.a.223 | Cl | Cl | I |
| I.a.224 | Br | Cl | I |
| I.a.225 | I | Cl | I |
| I.a.226 | CH₃ | Cl | I |
| I.a.227 | CF₃ | Cl | I |
| I.a.228 | SO₂CH₃ | Cl | I |
| I.a.229 | Cl | 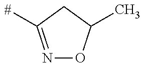 | I |
| I.a.230 | Br | 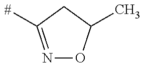 | I |
| I.a.231 | I | 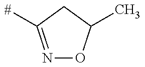 | I |
| I.a.232 | CH₃ | 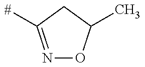 | I |
| I.a.233 | CF₃ | 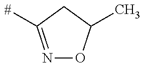 | I |
| I.a.234 | SO₂CH₃ | 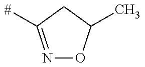 | I |
| I.a.235 | Cl | 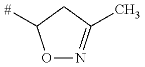 | I |
| I.a.236 | Br | 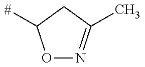 | I |
| I.a.237 | I | 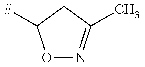 | I |
| I.a.238 | CH₃ | 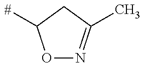 | I |
| I.a.239 | CF₃ | 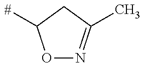 | I |
| I.a.240 | SO₂CH₃ | 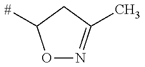 | I |
| I.a.241 | Cl | N(CH₃)SO₂CH₃ | I |
| I.a.242 | Br | N(CH₃)SO₂CH₃ | I |
| I.a.243 | I | N(CH₃)SO₂CH₃ | I |
| I.a.244 | CH₃ | N(CH₃)SO₂CH₃ | I |
| I.a.245 | CF₃ | N(CH₃)SO₂CH₃ | I |
| I.a.246 | SO₂CH₃ | N(CH₃)SO₂CH₃ | I |
| I.a.247 | Cl | CH₂OCH₂CH₂OCH₃ | I |
| I.a.248 | Br | CH₂OCH₂CH₂OCH₃ | I |
| I.a.249 | I | CH₂OCH₂CH₂OCH₃ | I |
| I.a.250 | CH₃ | CH₂OCH₂CH₂OCH₃ | I |
| I.a.251 | CF₃ | CH₂OCH₂CH₂OCH₃ | I |
| I.a.252 | SO₂CH₃ | CH₂OCH₂CH₂OCH₃ | I |
| I.a.253 | Cl | CH₂OCH₂CF₃ | I |
| I.a.254 | Br | CH₂OCH₂CF₃ | I |
| I.a.255 | I | CH₂OCH₂CF₃ | I |
| I.a.256 | CH₃ | CH₂OCH₂CF₃ | I |
| I.a.257 | CF₃ | CH₂OCH₂CF₃ | I |
| I.a.258 | SO₂CH₃ | CH₂OCH₂CF₃ | I |
| I.a.259 | Cl | 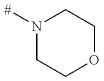 | 1 |
| I.a.260 | Br | 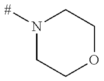 | I |
| I.a.261 | I | 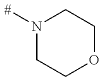 | I |
| I.a.262 | CH₃ | 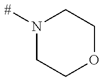 | I |
| I.a.263 | CF₃ | 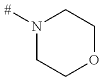 | I |
| I.a.264 | SO₂CH₃ | 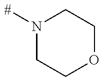 | I |
| I.a.265 | Cl | SO₂CH₃ | I |
| I.a.266 | Br | SO₂CH₃ | I |
| I.a.267 | I | SO₂CH₃ | I |
| I.a.268 | CH₃ | SO₂CH₃ | I |
| I.a.269 | CF₃ | SO₂CH₃ | I |
| I.a.270 | SO₂CH₃ | SO₂CH₃ | I |
| I.a.271 | Cl | H | CF₃ |
| I.a.272 | Br | H | CF₃ |
| I.a.273 | I | H | CF₃ |
| I.a.274 | CH₃ | H | CF₃ |
| I.a.275 | CF₃ | H | CF₃ |
| I.a.276 | SO₂CH₃ | H | CF₃ |
| I.a.277 | Cl | Cl | CF₃ |
| I.a.278 | Br | Cl | CF₃ |
| I.a.279 | I | Cl | CF₃ |
| I.a.280 | CH₃ | Cl | CF₃ |
| I.a.281 | CF₃ | Cl | CF₃ |
| I.a.282 | SO₂CH₃ | Cl | CF₃ |
| I.a.283 | Cl | 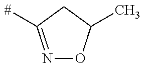 | CF₃ |
| I.a.284 | Br | 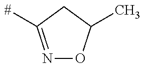 | CF₃ |
| I.a.285 | I | 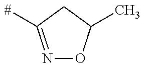 | CF₃ |
| I.a.286 | CH₃ | 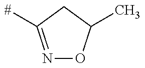 | CF₃ |
| I.a.287 | CF₃ | 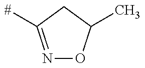 | CF₃ |
| I.a.288 | SO₂CH₃ | 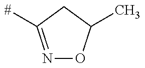 | CF₃ |
| I.a.289 | Cl | 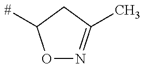 | CF₃ |
| I.a.290 | Br | 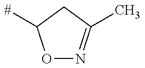 | CF₃ |
| I.a.291 | I | 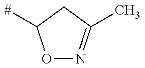 | CF₃ |
| I.a.292 | CH₃ | 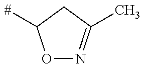 | CF₃ |
| I.a.293 | CF₃ | 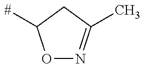 | CF₃ |
| I.a.294 | SO₂CH₃ | 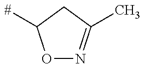 | CF₃ |
| I.a.295 | Cl | N(CH₃)SO₂CH₃ | CF₃ |
| I.a.296 | Br | N(CH₃)SO₂CH₃ | CF₃ |
| I.a.297 | I | N(CH₃)SO₂CH₃ | CF₃ |
| I.a.298 | CH₃ | N(CH₃)SO₂CH₃ | CF₃ |
| I.a.299 | CF₃ | N(CH₃)SO₂CH₃ | CF₃ |
| I.a.300 | SO₂CH₃ | N(CH₃)SO₂CH₃ | CF₃ |
| I.a.301 | Cl | CH₂OCH₂CH₂OCH₃ | CF₃ |
| I.a.302 | Br | CH₂OCH₂CH₂OCH₃ | CF₃ |
| I.a.303 | I | CH₂OCH₂CH₂OCH₃ | CF₃ |
| I.a.304 | CH₃ | CH₂OCH₂CH₂OCH₃ | CF₃ |
| I.a.305 | CF₃ | CH₂OCH₂CH₂OCH₃ | CF₃ |
| I.a.306 | SO₂CH₃ | CH₂OCH₂CH₂OCH₃ | CF₃ |
| I.a.307 | Cl | CH₂OCH₂CF₃ | CF₃ |
| I.a.308 | Br | CH₂OCH₂CF₃ | CF₃ |
| I.a.309 | I | CH₂OCH₂CF₃ | CF₃ |
| I.a.310 | CH₃ | CH₂OCH₂CF₃ | CF₃ |
| I.a.311 | CF₃ | CH₂OCH₂CF₃ | CF₃ |
| I.a.312 | SO₂CH₃ | CH₂OCH₂CF₃ | CF₃ |
| I.a.313 | Cl | 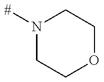 | CF₃ |
| I.a.314 | Br | 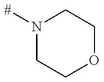 | CF₃ |
| I.a.315 | I | 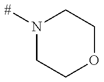 | CF₃ |
| I.a.316 | CH₃ | 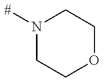 | CF₃ |
| I.a.317 | CF₃ | 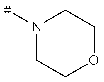 | CF₃ |
| I.a.318 | SO₂CH₃ | 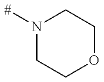 | CF₃ |
| I.a.319 | Cl | SO₂CH₃ | CF₃ |
| I.a.320 | Br | SO₂CH₃ | CF₃ |
| I.a.321 | I | SO₂CH₃ | CF₃ |
| I.a.322 | CH₃ | SO₂CH₃ | CF₃ |
| I.a.323 | CF₃ | SO₂CH₃ | CF₃ |
| I.a.324 | SO₂CH₃ | SO₂CH₃ | CF₃ |
| I.a.325 | Cl | H | OCH₃ |
| I.a.326 | Br | H | OCH₃ |
| I.a.327 | I | H | OCH₃ |
| I.a.328 | CH₃ | H | OCH₃ |
| I.a.329 | CF₃ | H | OCH₃ |
| I.a.330 | SO₂CH₃ | H | OCH₃ |
| I.a.331 | Cl | Cl | OCH₃ |
| I.a.332 | Br | Cl | OCH₃ |
| I.a.333 | I | Cl | OCH₃ |
| I.a.334 | CH₃ | Cl | OCH₃ |
| I.a.335 | CF₃ | Cl | OCH₃ |
| I.a.336 | SO₂CH₃ | Cl | OCH₃ |
| I.a.337 | Cl | 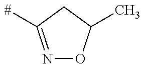 | OCH₃ |
| I.a.338 | Br | 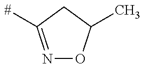 | OCH₃ |
| I.a.339 | I | 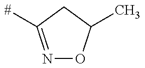 | OCH₃ |
| I.a.340 | CH₃ | 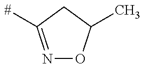 | OCH₃ |
| I.a.341 | CF₃ | 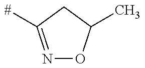 | OCH₃ |
| I.a.342 | SO₂CH₃ | 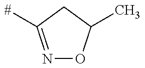 | OCH₃ |
| I.a.343 | Cl | 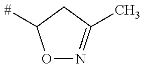 | OCH₃ |
| I.a.344 | Br | 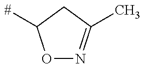 | OCH₃ |
| I.a.345 | I | 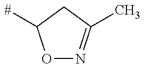 | OCH₃ |
| I.a.346 | CH₃ | 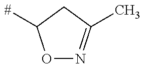 | OCH₃ |
| I.a.347 | CF₃ | 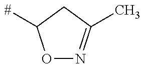 | OCH₃ |
| I.a.348 | SO₂CH₃ | 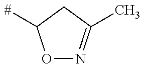 | OCH₃ |
| I.a.349 | Cl | N(CH₃)SO₂CH₃ | OCH₃ |
| I.a.350 | Br | N(CH₃)SO₂CH₃ | OCH₃ |
| I.a.351 | I | N(CH₃)SO₂CH₃ | OCH₃ |
| I.a.352 | CH₃ | N(CH₃)SO₂CH₃ | OCH₃ |
| I.a.353 | CF₃ | N(CH₃)SO₂CH₃ | OCH₃ |
| I.a.354 | SO₂CH₃ | N(CH₃)SO₂CH₃ | OCH₃ |
| I.a.355 | Cl | CH₂OCH₂CH₂OCH₃ | OCH₃ |
| I.a.356 | Br | CH₂OCH₂CH₂OCH₃ | OCH₃ |
| I.a.357 | I | CH₂OCH₂CH₂OCH₃ | OCH₃ |
| I.a.358 | CH₃ | CH₂OCH₂CH₂OCH₃ | OCH₃ |
| I.a.359 | CF₃ | CH₂OCH₂CH₂OCH₃ | OCH₃ |
| I.a.360 | SO₂CH₃ | CH₂OCH₂CH₂OCH₃ | OCH₃ |
| I.a.361 | Cl | CH₂OCH₂CF₃ | OCH₃ |
| I.a.362 | Br | CH₂OCH₂CF₃ | OCH₃ |
| I.a.363 | I | CH₂OCH₂CF₃ | OCH₃ |
| I.a.364 | CH₃ | CH₂OCH₂CF₃ | OCH₃ |
| I.a.365 | CF | CH₂OCH₂CF₃ | OCH₃ |
| I.a.366 | SO₂CH₃ | CH₂OCH₂CF₃ | OCH₃ |
| I.a.367 | Cl | 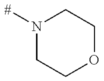 | OCH₃ |
| I.a.368 | Br | 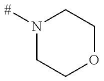 | OCH₃ |
| I.a.369 | I | 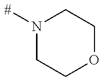 | OCH₃ |
| I.a.370 | CH₃ | 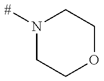 | OCH₃ |
| I.a.371 | CF₃ | 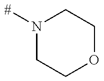 | OCH₃ |
| I.a.372 | SO₂CH₃ | 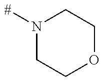 | OCH₃ |
| I.a.373 | Cl | SO₂CH₃ | OCH₃ |
| I.a.374 | Br | SO₂CH₃ | OCH₃ |
| I.a.375 | I | SO₂CH₃ | OCH₃ |
| I.a.376 | CH₃ | SO₂CH₃ | OCH₃ |
| I.a.377 | CF₃ | SO₂CH₃ | OCH₃ |
| I.a.378 | SO₂CH₃ | SO₂CH₃ | OCH₃ |
| I.a.379 | Cl | H | SO₂CH₃ |
| I.a.380 | Br | H | SO₂CH₃ |
| I.a.381 | I | H | SO₂CH₃ |
| I.a.382 | CH₃ | H | SO₂CH₃ |
| I.a.383 | CF₃ | H | SO₂CH₃ |
| I.a.384 | SO₂CH₃ | H | SO₂CH₃ |
| I.a.385 | Cl | Cl | SO₂CH₃ |
| I.a.386 | Br | Cl | SO₂CH₃ |
| I.a.387 | I | Cl | SO₂CH₃ |
| I.a.388 | CH₃ | Cl | SO₂CH₃ |
| I.a.389 | CF₃ | Cl | SO₂CH₃ |
| I.a.390 | SO₂CH₃ | Cl | SO₂CH₃ |
| I.a.391 | Cl | 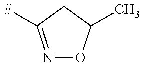 | SO₂CH₃ |
| I.a.392 | Br | 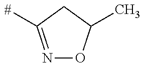 | SO₂CH₃ |
| I.a.393 | I | 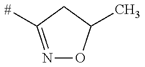 | SO₂CH₃ |
| I.a.394 | CH₃ | 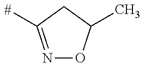 | SO₂CH₃ |
| I.a.395 | CF₃ | 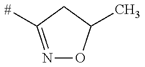 | SO₂CH₃ |
| I.a.396 | SO₂CH₃ | 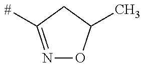 | SO₂CH₃ |
| I.a.397 | Cl | 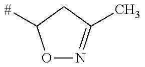 | SO₂CH₃ |
| I.a.398 | Br | 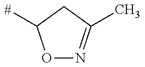 | SO₂CH₃ |
| I.a.399 | I | 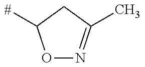 | SO₂CH₃ |
| I.a.400 | CH₃ | 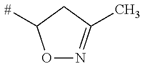 | SO₂CH₃ |
| I.a.401 | CF₃ | 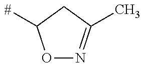 | SO₂CH₃ |
| I.a.402 | SO₂CH₃ | 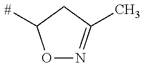 | SO₂CH₃ |
| I.a.403 | Cl | N(CH₃)SO₂CH₃ | SO₂CH₃ |
| I.a.404 | Br | N(CH₃)SO₂CH₃ | SO₂CH₃ |
| I.a.405 | I | N(CH₃)SO₂CH₃ | SO₂CH₃ |
| I.a.406 | CH₃ | N(CH₃)SO₂CH₃ | SO₂CH₃ |
| I.a.407 | CF₃ | N(CH₃)SO₂CH₃ | SO₂CH₃ |
| I.a.408 | SO₂CH₃ | N(CH₃)SO₂CH₃ | SO₂CH₃ |
| I.a.409 | Cl | CH₂OCH₂CH₂OCH₃ | SO₂CH₃ |
| I.a.410 | Br | CH₂OCH₂CH₂OCH₃ | SO₂CH₃ |
| I.a.411 | I | CH₂OCH₂CH₂OCH₃ | SO₂CH₃ |
| I.a.412 | CH₃ | CH₂OCH₂CH₂OCH₃ | SO₂CH₃ |
| I.a.413 | CF₃ | CH₂OCH₂CH₂OCH₃ | SO₂CH₃ |
| I.a.414 | SO₂CH₃ | CH₂OCH₂CH₂OCH₃ | SO₂CH₃ |
| I.a.415 | Cl | CH₂OCH₂CF₃ | SO₂CH₃ |
| I.a.416 | Br | CH₂OCH₂CF₃ | SO₂CH₃ |
| I.a.417 | I | CH₂OCH₂CF₃ | SO₂CH₃ |
| I.a.418 | CH₃ | CH₂OCH₂CF₃ | SO₂CH₃ |
| I.a.419 | CF₃ | CH₂OCH₂CF₃ | SO₂CH₃ |
| I.a.420 | SO₂CH₃ | CH₂OCH₂CF₃ | SO₂CH₃ |
| I.a.421 | Cl | 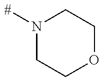 | SO₂CH₃ |
| I.a.422 | Br | 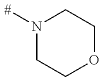 | SO₂CH₃ |
| I.a.423 | I | 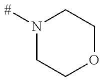 | SO₂CH₃ |
| I.a.424 | CH₃ | 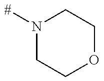 | SO₂CH₃ |
| I.a.425 | CF₃ | 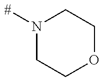 | SO₂CH₃ |
| I.a.426 | SO₂CH₃ | 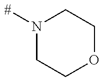 | SO₂CH₃ |
| I.a.427 | Cl | SO₂CH₃ | SO₂CH₃ |
| I.a.428 | Br | SO₂CH₃ | SO₂CH₃ |
| I.a.429 | I | SO₂CH₃ | SO₂CH₃ |
| I.a.430 | CH₃ | SO₂CH₃ | SO₂CH₃ |
| I.a.431 | CF₃ | SO₂CH₃ | SO₂CH₃ |
| I.a.432 | SO₂CH₃ | SO₂CH₃ | SO₂CH₃ |
| I.a.433 | Cl | H | CH₃ |
| I.a.434 | Br | H | CH₃ |
| I.a.435 | I | H | CH₃ |
| I.a.436 | CH₃ | H | CH₃ |
| I.a.437 | CF₃ | H | CH₃ |
| I.a.438 | SO₂CH₃ | H | CH₃ |
| I.a.439 | Cl | Cl | CH₃ |
| I.a.440 | Br | Cl | CH₃ |
| I.a.441 | I | Cl | CH₃ |
| I.a.442 | CH₃ | Cl | CH₃ |
| I.a.443 | CF₃ | Cl | CH₃ |
| I.a.444 | SO₂CH₃ | Cl | CH₃ |
| I.a.445 | Cl | 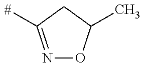 | CH₃ |
| I.a.446 | Br | 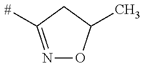 | CH₃ |
| I.a.447 | I | 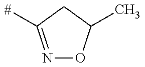 | CH₃ |
| I.a.448 | CH₃ | 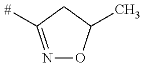 | CH₃ |
| I.a.449 | CF₃ | 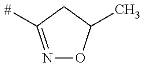 | CH₃ |
| I.a.450 | SO₂CH₃ | 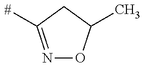 | CH₃ |
| I.a.451 | Cl | 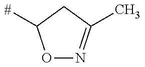 | CH₃ |
| I.a.452 | Br | 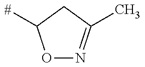 | CH₃ |
| I.a.453 | I | 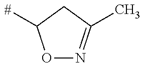 | CH₃ |
| I.a.454 | CH₃ | 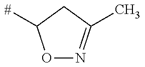 | CH₃ |
| I.a.455 | CF₃ | 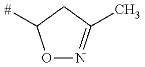 | CH₃ |
| I.a.456 | SO₂CH₃ | 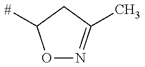 | CH₃ |
| I.a.457 | Cl | N(CH₃)SO₂CH₃ | CH₃ |
| I.a.458 | Br | N(CH₃)SO₂CH₃ | CH₃ |
| I.a.459 | I | N(CH₃)SO₂CH₃ | CH₃ |
| I.a.460 | CH | N(CH₃)SO₂CH₃ | CH₃ |
| I.a.461 | CF₃ | N(CH₃)SO₂CH₃ | CH₃ |
| I.a.462 | SO₂CH₃ | N(CH₃)SO₂CH₃ | CH₃ |
| I.a.463 | Cl | CH₂OCH₂CH₂OCH₃ | CH₃ |
| I.a.464 | Br | CH₂OCH₂CH₂OCH₃ | CH₃ |
| I.a.465 | I | CH₂OCH₂CH₂OCH₃ | CH₃ |
| I.a.466 | CH₃ | CH₂OCH₂CH₂OCH₃ | CH₃ |
| I.a.467 | CF₃ | CH₂OCH₂CH₂OCH₃ | CH₃ |
| I.a.468 | SO₂CH₃ | CH₂OCH₂CH₂OCH₃ | CH₃ |
| I.a.469 | Cl | CH₂OCH₂CF₃ | CH₃ |
| I.a.470 | Br | CH₂OCH₂CF₃ | CH₃ |
| I.a.471 | I | CH₂OCH₂CF₃ | CH₃ |
| I.a.472 | CH₃ | CH₂OCH₂CF₃ | CH₃ |
| I.a.473 | CF₃ | CH₂OCH₂CF₃ | CH₃ |
| I.a.474 | SO₂CH₃ | CH₂OCH₂CF₃ | CH₃ |
| I.a.475 | Cl | 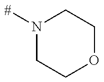 | CH₃ |
| I.a.476 | Br | 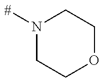 | CH₃ |
| I.a.477 | I | 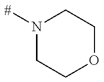 | CH₃ |
| I.a.478 | CH₃ | 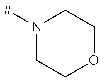 | CH₃ |
| I.a.479 | CF₃ | 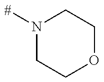 | CH₃ |
| I.a.480 | SO₂CH₃ | 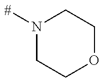 | CH₃ |
| I.a.481 | Cl | SO₂CH₃ | CH₃ |
| I.a.482 | Br | SO₂CH₃ | CH₃ |
| I.a.483 | I | SO₂CH₃ | CH₃ |
| I.a.484 | CH₃ | SO₂CH₃ | CH₃ |
| I.a.485 | CF₃ | SO₂CH₃ | CH₃ |
| I.a.486 | SO₂CH₃ | SO₂CH₃ | CH₃ |

The substituted pyridine compounds of formula I according to the invention can be prepared by standard processes of organic chemistry, for example by the following processes:

Picolinic acid derivatives of the formula II can be reacted with a thiol compound of the formula III to yield thioether compounds of the formula IV. In the formulae II and III, the variables have the meaning given for the compounds of formula I. The group X is a halogen atom, in particular Cl or Br. Y is a methyl or ethyl group.

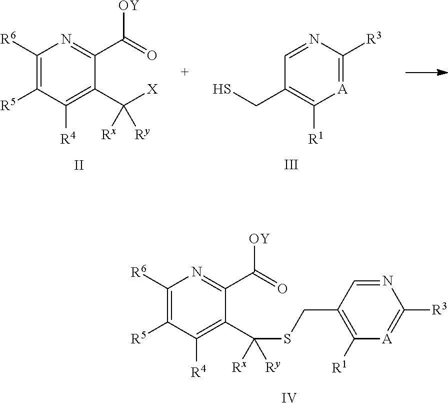

The reaction of the picolinic acid derivative II with the thiol compound III can be carried out according to literature procedures [cf. Journal of the Chemical Society, Perkin Transactions 1: Organic and Bio-Organic Chemistry (1972-1999) (1984), (7), 1501-1505] in an organic solvent, such as, for example acetonitrile or dimethylformamide (DMF), at temperatures between −78° C. and reflux of the solvent, preferably in a temperature range of from 10° C. to 50° C. It is also possible to use mixtures of the solvents mentioned. The starting materials II and III are generally reacted with one another in equimolar amounts.

The picolinic acid derivatives II can be prepared according to literature procedures (cf. Journal of Medicinal Chemistry, 32(4), 827-33; 1989).

The thiol compound III can be prepared from e.g. the corresponding thioacetate by cleavage with an alkali metal hydroxide like sodium hydroxide, potassium hydroxide or lithium hydroxide in water at a temperature of from 0° C. to 100° C., preferably at a temperature of from 10° C. to 30° C. Many benzylthiols can also be acquired from commercial sources. The thioacetate can be prepared from correspondingly substituted benzoic acids or halobenzenes on the basis of syntheses known in the literature [cf. Journal of Medicinal Chemistry 49(12), 3563-3580 (2006); Journal of Medicinal Chemistry 28(10), 1533-6 (1985); US 2004/077901; US 2004/068141; Chemistry—A European Journal 14(26), 7969-7977 (2008); Journal of Enzyme Inhibition and Medicinal Chemistry 17(3), 187-196 (2002)]. Suitably substituted benzoic acids and halobenzenes are known, for example from: WO 2002/006211, WO 2009/058237, WO 98/52926, WO 96/26193, EP-A 352 543, WO 98/52926, WO 97/30986, WO 98/12180.

The thioether compound IV can be reacted with an oxidizing agent to give the sulfone compound V.

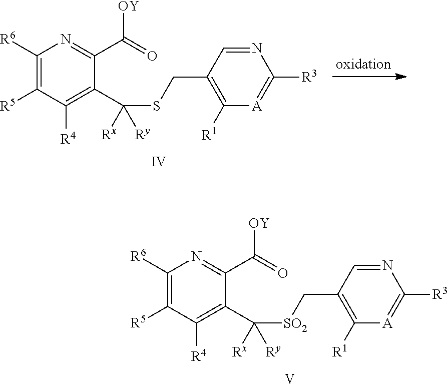

Suitable oxidizing agents include, for example, 3-chloroperoxybenzoic acid or hydrogen peroxide. The oxidation of the thioether compound IV to the sulfone compound V is usually carried out in an organic solvent, such as, for example methylene chloride, at a temperature of from 0° C. to reflux of the solvent, preferably at a temperature of from 10° C. to 25° C. The amount of the oxidizing agent is generally at least 2 molar equivalents relative to the thioether compound IV.

The sulfone compound V can be reacted with a base to give compounds of the formula I.4 (which correspond to compounds of the formula I with R=hydroxy).

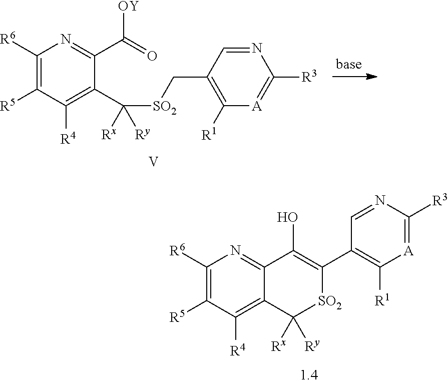

The cyclization reaction is usually carried out at a temperature of from −78° C. to 0° C., preferably at a temperature of from −60° C. to 0° C. in an inert organic solvent in the presence of a base (analogous to the procedure as described in WO 2010/000892).

Suitable inert organic solvents are tetrahydrofurane (THF), diethyl ether, diisopropyl ether and tert-butyl methyl ether, preferably tetrahydrofurane. It is also possible to use mixtures of the solvents mentioned. Suitable bases are lithiumdiisopropylamide, sodium tert-butoxide, potassium tert-butoxide, lithium tert-butoxide, sodium methoxide, potassium methoxide, lithium methoxide, triethylamine and tributylamine, preferably lithiumdiisopropylamide. The bases are generally employed in equimolar amounts; however, they can also be used in excess or, if appropriate, as solvents.

The hydroxy compound I.4 can be reacted with a base and an electrophile like an alkyl or acyl halide R^{A}—X (wherein X denotes a halogen atom, in particular Cl or Br) to give compounds of the formula I.5 (which correspond to compounds of the formula I with R═O—R^{A}).

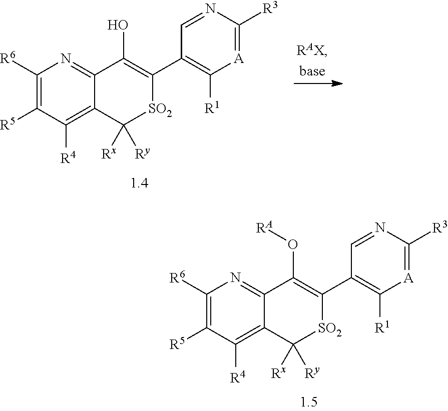

The reaction is usually carried out at a temperature of from −78° C. to 80° C., preferably at a temperature of from −60° C. to 0° C. in an inert organic solvent in the presence of a base. Suitable inert organic solvents are tetrahydrofurane (THF), diethyl ether, diisopropyl ether and tert-butyl methyl ether, preferably tetrahydrofurane. It is also possible to use mixtures of the solvents mentioned. Suitable bases are lithiumdiisopropylamide, sodium tert-butoxide, potassium tert-butoxide, lithium tert-butoxide, sodium methoxide, potassium methoxide, lithium methoxide, triethylamine and tributylamine, preferably lithiumdiisopropylamide. The bases are generally employed in equimolar amounts; however, they can also be used in excess or, if appropriate, as solvents.

In case the preparation of compounds I with R^{x} and/or R^{y}=halogen is desired, the compounds I.4 or I.5 which are substituted by hydrogen in the R^{x} and/or R^{y} positions can be deprotonated with a base, preferably lithiumdiisopropylamide, in an organic solvent like tetrahydrofurane, methl-tert-butylether or diethylether at a temperature of from −78° C. to 0° C., preferably at a temperature of from −60° C. to 0° C., and subsequently reacted with a halogenating agent like N-bromosuccinimide or N-fluorodi(benzenesulfonyl)amine at a temperature of from −78° C. to 0° C., preferably at a temperature or from −60° C. to 0° C.

In case the preparation of compounds I with R^{x} and/or R^{y}=alkyl or cycloalkyl is desired, the compounds I.4 or I.5 which are substituted by hydrogen in the R^{x} and/or R^{y} positions can be deprotonated with a base, preferably potassium-tertbutanolate, in an organic solvent like tetrahydrofurane, methl-tert-butylether or diethylether at a temperature of from −78° C. to 0° C., preferably at a temperature of from −60° C. to 0° C., and subsequently reacted with an alkylating agent like bromomethane or dibromoethane at a temperature of from −78° C. to 0° C., preferably at a temperature or from −60° C. to 0° C.

With respect to the variables, preferred embodiments of the intermediates II, III, IV and V correspond to those described above for the variables of the compound of formula I.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, the purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after application, for example in the case of the treatment of plants in the treated plant or in the harmful plant to be controlled.

As shown above the thioether compounds of formula IV are novel thioether compounds and suitable intermediates for the preparation of the compounds of formula I according to the present invention.

Therefore the present invention also provides novel thioether compounds of formula IV

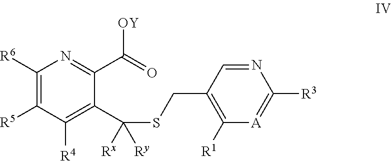

wherein the variables R¹, R³, R⁴, R⁵, R⁶, R^{x}, R^{y} and A have the same meaning given for the compound of formula I and Y is methyl or ethyl.

With respect to the variables R¹, R³, R⁴, R⁵, R⁶, R^{x}, R^{y} and A, preferred embodiments of the intermediate IV correspond to those described above for the variables of the compound of formula I.

As shown above the sulfone compounds of formula V are novel compounds and suitable intermediates for the preparation of the compounds of formula I according to the present invention.

Therefore the present invention also provides novel sulfone compounds of formula V

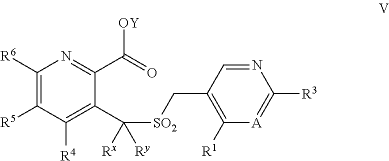

wherein the variables R¹, R³, R⁴, R⁵, R⁶, R^{x}, R^{y} and A have the same meaning given for the compound of formula I and Y is methyl or ethyl.

With respect to the variables R¹, R³, R⁴, R⁵, R⁶, R^{x}, R^{y} and A, preferred embodiments of the intermediate V correspond to those described above for the variables of the compound of formula I.

As shown above the compounds of formula VI are novel compounds and suitable intermediates for the preparation of the compounds of formula I according to the present invention.

The substituted pyridine compounds of formula I are suitable as herbicides. They are suitable as such or as an appropriately formulated composition (agrochemical composition). As used in this application, the terms “formulated composition” and “herbicidal composition”/“composition” are synonyms.

The herbicidal compositions comprising the compounds of formula I control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds I or compositions comprising them can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are the following: Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera and Zea mays.

The compounds of formula I according to the invention can also be used in genetically modified plants. The term “genetically modified plants” is to be understood as plants whose genetic material has been modified by the use of recombinant DNA techniques to include an inserted sequence of DNA that is not native to that plant species' genome or to exhibit a deletion of DNA that was native to that species' genome, wherein the modification(s) cannot readily be obtained by cross breeding, mutagenesis or natural recombination alone. Often, a particular genetically modified plant will be one that has obtained its genetic modification(s) by inheritance through a natural breeding or propagation process from an ancestral plant whose genome was the one directly treated by use of a recombinant DNA technique. Typically, one or more genes have been integrated into the genetic material of a genetically modified plant in order to improve certain properties of the plant. Such genetic modifications also include but are not limited to targeted post-translational modification of protein(s), oligo- or polypeptides. e.g., by inclusion therein of amino acid mutation(s) that permit, decrease, or promote glycosylation or polymer additions such as prenylation, acetylation farnesylation, or PEG moiety attachment.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxyphenylpyruvate dioxygenase (HPPD) inhibitors or phytoene desaturase (PDS) inhibitors; acetolactate synthase (ALS) inhibitors such as sulfonyl ureas or imidazolinones; enolpyruvyl shikimate 3-phosphate synthase (EPSP) inhibitors such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i.e. bromoxynil or ioxynil) herbicides as a result of conventional methods of breeding or genetic engineering; furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are, for example, described in Pest Management Science 61, 2005, 246; 61,2005, 258; 61,2005, 277; 61,2005, 269; 61,2005, 286; 64, 2008, 326; 64, 2008, 332; Weed Science 57, 2009, 108; Australian Journal of Agricultural Research 58, 2007, 708; Science 316, 2007, 1185; and references quoted therein. Several cultivated plants have been rendered tolerant to herbicides by mutgenesis and conventional methods of breeding, e. g., Clearfield® summer rape (Canola, BASF SE, Germany) being tolerant to imidazolinones, e. g., imazamox, or ExpressSun® sunflowers (DuPont, USA) being tolerant to sulfonyl ureas, e.g., tribenuron. Genetic engineering methods have been used to render cultivated plants such as soybean, cotton, corn, beets and rape, tolerant to herbicides such as glyphosate, imidazolinones and glufosinate, some of which are under development or commercially available under the brands or trade names RoundupReady® (glyphosate tolerant, Monsanto, USA), Cultivance® (imidazolinone tolerant, BASF SE, Germany) and LibertyLink® (glufosinate tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as delta-endotoxins, e.g., CryIA(b), CryIA(c), CryIF, CryIF(a2), CryIIA(b), CryIIIA, CryIIIB(b1) or Cry9c; vegetative insecticidal proteins (VIP), e.g., VIP1, VIP2, VIP3 or VIP3A; insecticidal proteins of bacteria colonizing nematodes, e.g., Photorhabdus spp. or Xenorhabdus spp.; toxins produced by animals, such as scorpion toxins, arachnid toxins, wasp toxins, or other insect-specific neurotoxins; toxins produced by fungi, such as Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as including pre-toxins, hybrid proteins, truncated or otherwise modified proteins. Hybrid proteins are characterized by a new combination of protein domains, (see, e.g., WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e.g., in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e.g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e.g., Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g., EP-A 392 225), plant disease resistance genes (e.g., potato culti-vars, which express resistance genes acting against Phytophthora infestans derived from the Mexican wild potato, Solanum bulbocastanum) or T4-lyso-zym (e.g., potato cultivars capable of synthesizing these proteins with increased resistance against bacteria such as Erwinia amylovora). The methods for producing such genetically modified plants are generally known to the person skilled in the art and are described, e.g., in the publications mentioned above.

Furthermore, plants are also covered that are by the use of recombinant DNA techniques capable to synthesize one or more proteins to increase the productivity (e.g., bio-mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of ingredients or new ingredients, specifically to improve human or animal nutrition, e.g., oil crops that produce health-promoting long-chain omega-3 fatty acids or unsaturated omega-9 fatty acids (e.g., Nexera® rape, Dow AgroSciences, Canada).

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

Furthermore, it has been found that the compounds of formula I are also suitable for the defoliation and/or desiccation of plant parts, for which crop plants such as cotton, potato, oilseed rape, sunflower, soybean or field beans, in particular cotton, are suitable. In this regard, compositions for the desiccation and/or defoliation of plants, processes for preparing these compositions and methods for desiccating and/or defoliating plants using the compounds of the formula I have been found.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

The invention also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling unwanted plants, especially for controlling unwanted plants in cultivated plants and which does not result in a substantial damage to the treated plants. Such an amount can vary in a broad range and is dependent on various factors, such as the plants to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for agrochemical composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further agrochemical compositions types are defined in the “Catalogue of pesticide formulation types and international coding system”, Technical Monograph No. 2, 6^{th} Ed. May 2008, CropLife International.

The agrochemical compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for agrochemical composition types and their preparation are:

i) Water-soluble concentrates (SL, LS)

10-60 wt % of a compound of formula I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible concentrates (DC)

5-25 wt % of a compound of formula I according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable concentrates (EC)

15-70 wt % of compound of formula I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of compound of formula I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound of formula I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-dispersible granules and water-soluble granules (WG, SG)

50-80 wt % of a compound of formula I according to the invention are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.

vii) Water-dispersible powders and water-soluble powders (WP, SP, WS)

50-80 wt % of a compound of formula I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound of formula I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

iv) Microemulsion (ME)

5-20 wt % of a compound of formula I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.

iv) Microcapsules (CS)

An oil phase comprising 5-50 wt % of a compound of formula I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition.

ix) Dustable powders (DP, DS)

1-10 wt % of a compound of formula I according to the invention are ground finely and mixed intimately with solid carrier (e.g. finely divided kaolin) ad 100 wt %.

x) Granules (GR, FG)

0.5-30 wt % of a compound of formula I according to the invention is ground finely and associated with solid carrier (e.g. silicate) ad 100 wt %. Granulation is achieved by extrusion, spray-drying or the fluidized bed.

xi) Ultra-low volume liquids (UL)

1-50 wt % of a compound of formula I according to the invention are dissolved in organic solvent (e.g. aromatic hydrocarbon) ad 100 wt %.

The agrochemical compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha and in particular from 0.1 to 0.75 kg per ha.

In another preferred embodiment of the invention, the rates of application of the compound of formula I according to the present invention (total amount of the compound of formula I) are from 0.1 g/ha to 3000 g/ha, preferably 10 g/ha to 1000 g/ha of active substance (a.s.), depending on the control target, the season, the target plants and the growth stage.

In another preferred embodiment of the invention, the application rates of the compound of formula I are in the range from 0.1 g/ha to 5000 g/ha and preferably in the range from 1 g/ha to 2500 g/ha or from 5 g/ha to 2000 g/ha of active substance (a.s.).

In another preferred embodiment of the invention, the application rate of the compound of formula I is 0.1 to 1000 g/ha, preferably 1 to 750 g/ha, more preferably 5 to 500 g/ha, of active substance.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

In another preferred embodiment of the invention, to treat the seed, the compounds I are generally employed in amounts of from 0.001 to 10 kg per 100 kg of seed.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the agrochemical composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the agrochemical composition according to the invention such as parts of a kit or parts of a binary or ternary mixture may be mixed by the user himself in a spray tank and further auxiliaries may be added, if appropriate.

In a further embodiment, either individual components of the agrochemical composition according to the invention or partially premixed components, e.g. components comprising compounds I and/or active substances from the groups b1) to b15), may be mixed by the user in a spray tank and further auxiliaries and additives may be added, if appropriate.

In a further embodiment, either individual components of the agrochemical composition according to the invention or partially premixed components, e.g. components comprising compounds I and/or active substances from the groups b1) to b15), can be applied jointly (e.g. after tank mix) or consecutively.

The compounds of formula I or the herbicidal compositions comprising them can be applied pre-, post-emergence or pre-plant, or together with the seed of a crop plant. It is also possible to apply the herbicidal composition or active compounds by applying seed, pretreated with the herbicidal compositions or active compounds, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the compounds of formula I or the herbicidal compositions can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of formula I according to the invention or the compositions prepared therefrom. Here, the herbicidal compositions can be applied diluted or undiluted.

The term “seed” comprises seed of all types, such as, for example, corns, seeds, fruits, tubers, seedlings and similar forms. Here, preferably, the term seed describes corns and seeds. The seed used can be seed of the useful plants mentioned above, but also the seed of transgenic plants or plants obtained by customary breeding methods.

To widen the spectrum of action and to achieve synergistic effects, the compounds of formula I may be mixed with a large number of representatives of other herbicidal or growth-regulating active ingredient groups and then applied concomitantly. Suitable components for mixtures are, for example, herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

It may furthermore be beneficial to apply the compounds of formula I alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

Moreover, it may be useful to apply the compounds of formula I in combination with safeners. Safeners are chemical compounds which prevent or reduce damage on useful plants without having a major impact on the herbicidal action of the compounds of the formula I towards unwanted plants. They can be applied either before sowings (e.g. on seed treatments, shoots or seedlings) or in the pre-emergence application or post-emergence application of the useful plant. The safeners and the compounds of formula I can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

In one embodiment of the present invention the compositions according to the present invention comprise at least one compound of formula I and at least one further active compound B (herbicide B).

The further active compound B is preferably selected from the herbicides of class b1) to b15):

b1) lipid biosynthesis inhibitors;

b2) acetolactate synthase inhibitors (ALS inhibitors);

b3) photosynthesis inhibitors;

b4) protoporphyrinogen-IX oxidase inhibitors,

b5) bleacher herbicides;

b6) enolpyruvyl shikimate 3-phosphate synthase inhibitors (EPSP inhibitors);

b7) glutamine synthetase inhibitors;

b8) 7,8-dihydropteroate synthase inhibitors (DHP inhibitors);

b9) mitosis inhibitors;

b10) inhibitors of the synthesis of very long chain fatty acids (VLCFA inhibitors);

b11) cellulose biosynthesis inhibitors;

b12) decoupler herbicides;
b13) auxinic herbicides;
b14) auxin transport inhibitors; and

b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters; including their agriculturally acceptable salts or derivatives.

According to another embodiment of the invention the compositions contain at least one inhibitor of the lipid biosynthesis (herbicide b1). These are compounds that inhibit lipid biosynthesis. Inhibition of the lipid biosynthesis can be affected either through inhibition of acetylCoA carboxylase (hereinafter termed ACC herbicides) or through a different mode of action (hereinafter termed non-ACC herbicides). The ACC herbicides belong to the group A of the HRAC classification system whereas the non-ACC herbicides belong to the group N of the HRAC classification.

According to another embodiment of the invention the compositions contain at least one ALS inhibitor (herbicide b2). The herbicidal activity of these compounds is based on the inhibition of acetolactate synthase and thus on the inhibition of the branched chain amino acid biosynthesis. These inhibitors belong to the group B of the HRAC classification system.

According to another embodiment of the invention the compositions contain at least one inhibitor of photosynthesis (herbicide b3). The herbicidal activity of these compounds is based either on the inhibition of the photosystem II in plants (so-called PSII inhibitors, groups C₁, C₂ and C₃ of HRAC classification) or on diverting the electron transfer in photosystem I in plants (so-called PSI inhibitors, group D of HRAC classification) and thus on an inhibition of photosynthesis. Amongst these, PSII inhibitors are preferred.

According to another embodiment of the invention the compositions contain at least one inhibitor of protoporphyrinogen-IX-oxidase (herbicide b4). The herbicidal activity of these compounds is based on the inhibition of the protoporphyrinogen-IX-oxidase. These inhibitors belong to the group E of the HRAC classification system.

According to another embodiment of the invention the compositions contain at least one bleacher-herbicide (herbicide b5). The herbicidal activity of these compounds is based on the inhibition of the carotenoid biosynthesis. These include compounds which inhibit carotenoid biosynthesis by inhibition of phytoene desaturase (so-called PDS inhibitors, group F1 of HRAC classification), compounds that inhibit the 4-hydroxyphenylpyruvate-dioxygenase (HPPD inhibitors, group F2 of HRAC classification), compounds that inhibit DOXsynthase (group F4 of HRAC class) and compounds which inhibit carotenoid biosynthesis by an unknown mode of action (bleacher—unknown target, group F3 of HRAC classification).

According to another embodiment of the invention the compositions contain at least one EPSP synthase inhibitor (herbicide b6). The herbicidal activity of these compounds is based on the inhibition of enolpyruvyl shikimate 3-phosphate synthase, and thus on the inhibition of the amino acid biosynthesis in plants. These inhibitors belong to the group G of the HRAC classification system.

According to another embodiment of the invention the compositions contain at least one glutamine synthetase inhibitor (herbicide b7). The herbicidal activity of these compounds is based on the inhibition of glutamine synthetase, and thus on the inhibition of the amino acid biosynthesis in plants. These inhibitors belong to the group H of the HRAC classification system.

According to another embodiment of the invention the compositions contain at least one DHP synthase inhibitor (herbicide b8). The herbicidal activity of these compounds is based on the inhibition of 7,8-dihydropteroate synthase. These inhibitors belong to the group I of the HRAC classification system.

According to another embodiment of the invention the compositions contain at least one mitosis inhibitor (herbicide b9). The herbicidal activity of these compounds is based on the disturbance or inhibition of microtubule formation or organization, and thus on the inhibition of mitosis. These inhibitors belong to the groups K1 and K2 of the HRAC classification system. Among these, compounds of the group K1, in particular dinitroanilines, are preferred.

According to another embodiment of the invention the compositions contain at least one VLCFA inhibitor (herbicide b10). The herbicidal activity of these compounds is based on the inhibition of the synthesis of very long chain fatty acids and thus on the disturbance or inhibition of cell division in plants. These inhibitors belong to the group K3 of the HRAC classification system.

According to another embodiment of the invention the compositions contain at least one cellulose biosynthesis inhibitor (herbicide b11). The herbicidal activity of these compounds is based on the inhibition of the biosynthesis of cellulose and thus on the inhibition of the synthesis of cell walls in plants. These inhibitors belong to the group L of the HRAC classification system.

According to another embodiment of the invention the compositions contain at least one decoupler herbicide (herbicide b12). The herbicidal activity of these compounds is based on the disruption of the cell membrane. These inhibitors belong to the group M of the HRAC classification system.

According to another embodiment of the invention the compositions contain at least one auxinic herbicide (herbicide b13). These include compounds that mimic auxins, i.e. plant hormones, and affect the growth of the plants. These compounds belong to the group O of the HRAC classification system.

According to another embodiment of the invention the compositions contain at least one auxin transport inhibitor (herbicide b14). The herbicidal activity of these compounds is based on the inhibition of the auxin transport in plants. These compounds belong to the group P of the HRAC classification system.

As to the given mechanisms of action and classification of the active substances, see e.g. “HRAC, Classification of Herbicides According to Mode of Action”, http://www.plantprotection.org/hrac/MOA.html).

Preference is given to those compositions according to the present invention comprising at least one herbicide B selected from herbicides of class b2, b3, b4, b5, b6, b9 and b10.

Specific preference is given to those compositions according to the present invention which comprise at least one herbicide B selected from the herbicides of class b4, b6 b9 and b10.

Particular preference is given to those compositions according to the present invention which comprise at least one herbicide B selected from the herbicides of class b4, b6 and b10.

Examples of herbicides B which can be used in combination with the compounds of formula I according to the present invention are:

b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron,

imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,

pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),

sulfonylaminocarbonyl-triazolinone herbicides such as flucarbazone, flucarbazone-sodium, propoxycarbazone, propoxycarbazone-sodium, thiencarbazone and thiencarbazone-methyl; and triafamone;

among these, a preferred embodiment of the invention relates to those compositions comprising at least one imidazolinone herbicide;

b3) from the group of the photosynthesis inhibitors:

amicarbazone, inhibitors of the photosystem II, e.g. triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, ethyl[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-11-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-11-carboxamide (CAS 452099-05-7), N-tetrahydrofurfuryl-3-(2-chloro-6-fluoro-4-trifluoromethylphenoxy)-5-methyl-1H-pyrazole-11-carboxamide (CAS 45100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione, 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione, 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione and (Z)-4-[2-Chloro-5-(4-chloro-5-difluoromethoxy-1-methyl-1H-pyrazol-3-yl)-4-fluoro-phenoxy]-3-methyl-but-2-enoic acid methyl ester;

b5) from the group of the bleacher herbicides:

PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, clomazone, isoxaflutole, mesotrione, pyrasulfotole, pyrazolynate, pyrazoxyfen, sulcotrione, tefuryltrione, tembotrione, topramezone and bicyclopyrone, bleacher, unknown target: aclonifen, amitrole and flumeturon;

b6) from the group of the EPSP synthase inhibitors:

glyphosate, glyphosate-isopropylammonium, glyposate-potassium and glyphosate-trimesium (sulfosate);

b7) from the group of the glutamine synthase inhibitors:

bilanaphos (bialaphos), bilanaphos-sodium, glufosinate, glufosinate-P and glufosinate-ammonium;

b8) from the group of the DHP synthase inhibitors:

asulam;

b9) from the group of the mitosis inhibitors:

compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: chlorpropham, propham and carbetamide, among these, compounds of group K1, in particular dinitroanilines are preferred;

b10) from the group of the VLCFA inhibitors:

chloroacetamides such as acetochlor, alachlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide and napropamide, tetrazolinones such fentrazamide, and other herbicides such as anilofos, cafenstrole, fenoxasulfone, ipfencarbazone, piperophos, pyroxasulfone and isoxazoline compounds of the formulae II.1, II.2, II.3, II.4, II.5, II.6, II.7, II.8 and II.9

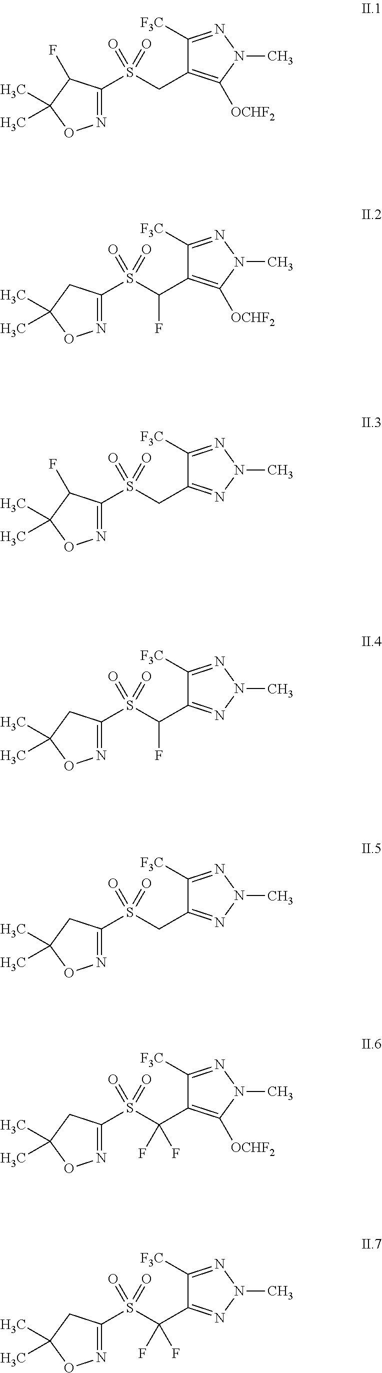

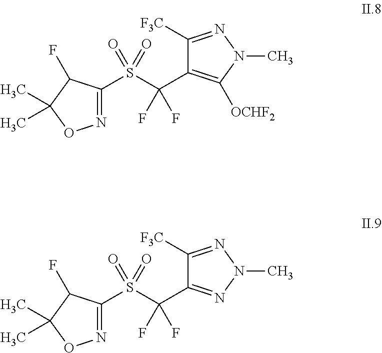

the isoxazoline compounds of the formulae II.1 to II.9 as shown above are known in the art, e.g. from WO 2006/024820, WO 2006/037945, WO 2007/071900 and WO 2007/096576;

among the VLCFA inhibitors, preference is given to chloroacetamides and oxyacetamides;

b11) from the group of the cellulose biosynthesis inhibitors:

chlorthiamid, dichlobenil, flupoxam, isoxaben and 1-Cyclohexyl-5-pentafluorphenyloxy-1⁴-[1,2,4,6]thiatriazin-3-ylamine;

b12) from the group of the decoupler herbicides:

dinoseb, dinoterb and DNOC and its salts;

b13) from the group of the auxinic herbicides:

2,4-D and its salts and esters, 2,4-DB and its salts and esters, aminopyralid and its salts such as aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, aminocyclopyrachlor and its salts and esters, and 4-Amino-3-chloro-6-(4-chloro-2-fluoro-3-methoxyphenyl)-2-pyridinecarboxylic acid (CAS 943832-60-8);

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters.

Preferred herbicides B that can be used in combination with the compounds of the formula I according to the present invention are:

b1) from the group of the lipid biosynthesis inhibitors:

clethodim, clodinafop-propargyl, cycloxydim, cyhalofop-butyl, diclofop-methyl, fenoxaprop-P-ethyl, fluazifop-P-butyl, haloxyfop-P-methyl, metamifop, pinoxaden, profoxydim, propaquizafop, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, dimepiperate, EPTC, esprocarb, ethofumesate, molinate, orbencarb, prosulfocarb, thiobencarb and triallate;

b2) from the group of the ALS inhibitors:

amidosulfuron, azimsulfuron, bensulfuron-methyl, bispyribac-sodium, chlorimuron-ethyl, chlorsulfuron, cloransulam-methyl, cyclosulfamuron, diclosulam, ethametsulfuron-methyl, ethoxysulfuron, flazasulfuron, florasulam, flucarbazone-sodium, flucetosulfuron, flumetsulam, flupyrsulfuron-methyl-sodium, foramsulfuron, halosulfuron-methyl, imazamethabenz-methyl, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, metosulam, metsulfuron-methyl, nicosulfuron, orthosulfamuron, oxasulfuron, penoxsulam, primisulfuron-methyl, propoxycarbazon-sodium, propyrisulfuron, prosulfuron, pyrazosulfuron-ethyl, pyribenzoxim, pyrimisulfan, pyriftalid, pyriminobac-methyl, pyrithiobac-sodium, pyroxsulam, rimsulfuron, sulfometuron-methyl, sulfosulfuron, thiencarbazone-methyl, thifensulfuron-methyl, triasulfuron, tribenuron-methyl, trifloxysulfuron, triflusulfuron-methyl, tritosulfuron and triafamone;

b3) from the group of the photosynthesis inhibitors:

ametryn, amicarbazone, atrazine, bentazone, bentazone-sodium, bromoxynil and its salts and esters, chloridazone, chlorotoluron, cyanazine, desmedipham, diquat-dibromide, diuron, fluometuron, hexazinone, ioxynil and its salts and esters, isoproturon, lenacil, linuron, metamitron, methabenzthiazuron, metribuzin, paraquat, paraquat-dichloride, phenmedipham, propanil, pyridate, simazine, terbutryn, terbuthylazine and thidiazuron;

b4) from the group of the protoporphyrinogen-IX oxidase inhibitors:

acifluorfen-sodium, bencarbazone, benzfendizone, butafenacil, carfentrazone-ethyl, cinidon-ethyl, flufenpyr-ethyl, flumiclorac-pentyl, flumioxazin, fluoroglycofen-ethyl, fomesafen, lactofen, oxadiargyl, oxadiazon, oxyfluorfen, pentoxazone, pyraflufen-ethyl, saflufenacil, sulfentrazone, ethyl[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-11-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-11-carboxamide (CAS 452099-05-7), N-tetrahydrofurfuryl-3-(2-chloro-6-fluoro-4-trifluoromethylphenoxy)-5-methyl-1H-pyrazole-11-carboxamide (CAS 45100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione, 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione and 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione;

b5) from the group of the bleacher herbicides:

aclonifen, beflubutamid, benzobicyclon, clomazone, diflufenican, flurochloridone, flurtamone, isoxaflutole, mesotrione, norflurazon, picolinafen, pyrasulfotole, pyrazolynate, sulcotrione, tefuryltrione, tembotrione, topramezone, bicyclopyrone, 4-(3-trifluoromethylphenoxy)-2-(4-trifluoromethylphenyl)pyrimidine (CAS 180608-33-7), amitrole and flumeturon;

b6) from the group of the EPSP synthase inhibitors:

glyphosate, glyphosate-isopropylammonium, glyphosate-potassium and glyphosate-trimesium (sulfosate);

b7) from the group of the glutamine synthase inhibitors:

glufosinate, glufosinate-P, glufosinate-ammonium;

b8) from the group of the DHP synthase inhibitors: asulam;

b9) from the group of the mitosis inhibitors:

benfluralin, dithiopyr, ethalfluralin, oryzalin, pendimethalin, thiazopyr and trifluralin;

b10) from the group of the VLCFA inhibitors:

acetochlor, alachlor, anilofos, butachlor, cafenstrole, dimethenamid, dimethenamid-P, fentrazamide, flufenacet, mefenacet, metazachlor, metolachlor, S-metolachlor, naproanilide, napropamide, pretilachlor, fenoxasulfone, ipfencarbazone, pyroxasulfone thenylchlor and isoxazoline compounds of the formulae II.1, II.2, II.3, II.4, II.5, II.6, II.7, II.8 and II.9 as mentioned above;

b11) from the group of the cellulose biosynthesis inhibitors: dichlobenil, flupoxam, isoxaben and 1-Cyclohexyl-5-pentafluorphenyloxy-1⁴-[1,2,4,6]thiatriazin-3-ylamine;

b13) from the group of the auxinic herbicides:

2,4-D and its salts and esters, aminopyralid and its salts such as aminopyralid-tris(2-hydroxypropyl)ammonium and its esters, clopyralid and its salts and esters, dicamba and its salts and esters, dichlorprop-P and its salts and esters, fluroxypyr-meptyl, MCPA and its salts and esters, MCPB and its salts and esters, mecoprop-P and its salts and esters, picloram and its salts and esters, quinclorac, quinmerac, triclopyr and its salts and esters, and aminocyclopyrachlor and its salts and esters;

b14) from the group of the auxin transport inhibitors: diflufenzopyr and diflufenzopyr-sodium;

b15) from the group of the other herbicides: bromobutide, cinmethylin, cumyluron, dalapon, difenzoquat, difenzoquat-metilsulfate, DSMA, dymron (=daimuron), flamprop, flamprop-isopropyl, flamprop-methyl, flamprop-M-isopropyl, flamprop-M-methyl, indanofan, indaziflam, metam, methylbromide, MSMA, oxaziclomefone, pyributicarb, triaziflam, tridiphane and 6-chloro-3-(2-cyclopropyl-6-methylphenoxy)-4-pyridazinol (CAS 499223-49-3) and its salts and esters.

Particularly preferred herbicides B that can be used in combination with the compounds of the formula I according to the present invention are:

b1) from the group of the lipid biosynthesis inhibitors: clodinafop-propargyl, cycloxydim, cyhalofop-butyl, fenoxaprop-P-ethyl, pinoxaden, profoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); esprocarb, prosulfocarb, thiobencarb and triallate;

b2) from the group of the ALS inhibitors: bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, nicosulfuron, penoxsulam, propoxycarbazon-sodium, propyrisulfuron, pyrazosulfuron-ethyl, pyroxsulam, rimsulfuron, sulfosulfuron, thiencarbazon-methyl, tritosulfuron and triafamone;

b3) from the group of the photosynthesis inhibitors: ametryn, atrazine, diuron, fluometuron, hexazinone, isoproturon, linuron, metribuzin, paraquat, paraquat-dichloride, propanil, terbutryn and terbuthylazine;

b4) from the group of the protoporphyrinogen-IX oxidase inhibitors: flumioxazin, oxyfluorfen, saflufenacil, sulfentrazone, ethyl[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione and 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione, and 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione;

b5) from the group of the bleacher herbicides: clomazone, diflufenican, flurochloridone, isoxaflutole, mesotrione, picolinafen, sulcotrione, tefuryltrione, tembotrione, topramezone, bicyclopyrone, amitrole and flumeturon;

b6) from the group of the EPSP synthase inhibitors: glyphosate, glyphosate-isopropylammonium and glyphosate-trimesium (sulfosate);

b7) from the group of the glutamine synthase inhibitors: glufosinate, glufosinate-P and glufosinate-ammonium;

b9) from the group of the mitosis inhibitors: pendimethalin and trifluralin;

b10) from the group of the VLCFA inhibitors: acetochlor, cafenstrole, dimethenamid-P, fentrazamide, flufenacet, mefenacet, metazachlor, metolachlor, S-metolachlor, fenoxasulfone, ipfencarbazone and pyroxasulfone; likewise, preference is given to isoxazoline compounds of the formulae II.1, II.2, II.3, II.4, II.5, II.6, II.7, II.8 and II.9 as mentioned above;

b11) from the group of the cellulose biosynthesis inhibitors: isoxaben;

b13) from the group of the auxinic herbicides: 2,4-D and its salts and esters, aminopyralid and its salts and its esters, clopyralid and its salts and esters, dicamba and its salts and esters, fluroxypyr-meptyl, quinclorac, quinmerac and aminocyclopyrachlor and its salts and esters;

b14) from the group of the auxin transport inhibitors: diflufenzopyr and diflufenzopyr-sodium,

b15) from the group of the other herbicides: dymron (=daimuron), indanofan, indaziflam, oxaziclomefone and triaziflam.

In another embodiment of the present invention the compositions according to the present invention comprise at least one compound of formula I and at least one safener C.

Safeners are chemical compounds which prevent or reduce damage on useful plants without having a major impact on the herbicidal action of the herbicidal active components of the present compositions towards unwanted plants. They can be applied either before sowings (e.g. on seed treatments, shoots or seedlings) or in the pre-emergence application or post-emergence application of the useful plant. The safeners and the compounds of formula I and/or the herbicides B can be applied simultaneously or in succession.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

Particularly preferred safeners C are benoxacor, cloquintocet, cyprosulfamide, dichlormid, fenchlorazole, fenclorim, furilazole, isoxadifen, mefenpyr, naphtalic anhydride, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

The active compounds B of groups b1) to b15) and the active compounds C are known herbicides and safeners, see, for example, The Compendium of Pesticide Common Names (http://www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

If the herbicides B and/or the safeners C are capable of forming geometrical isomers, for example E/Z isomers, both the pure isomers and mixtures thereof may be used in the compositions according to the invention.

If the herbicides B and/or the safeners C have one of more centers of chirality and are thus present as enantiomers or diastereomers, both the pure enantiomers and diastereomers and mixtures thereof may be used in the compositions according to the invention.

If the herbicides B and/or the safeners C have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Active compounds B and C having a carboxyl group can be employed in the form of the acid, in the form of an agriculturally suitable salt as mentioned above or else in the form of an agriculturally acceptable derivative in the compositions according to the invention, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

In the case of dicamba, suitable salts include those, where the counterion is an agriculturally acceptable cation. For example, suitable salts of dicamba are dicamba-sodium, dicamba-potassium, dicamba-methylammonium, dicamba-dimethylammonium, dicamba-isopropylammonium, dicamba-diglycolamine, dicamba-olamine, dicamba-diolamine, dicamba-trolamine, dicamba-N,N-bis-(3-aminopropyl)methylamine and dicamba-diethylenetriamine. Examples of a suitable ester are dicamba-methyl and dicamba-butotyl.

Suitable salts of 2,4-D are 2,4-D-ammonium, 2,4-D-dimethylammonium, 2,4-D-diethylammonium, 2,4-D-diethanolammonium (2,4-D-diolamine), 2,4-D-triethanolammonium, 2,4-D-isopropylammonium, 2,4-D-triisopropanolammonium, 2,4-D-heptylammonium, 2,4-D-dodecylammonium, 2,4-D-tetradecylammonium, 2,4-D-triethylammonium, 2,4-D-tris(2-hydroxypropyl)ammonium, 2,4-D-tris(isopropyl)ammonium, 2,4-D-trolamine, 2,4-D-lithium, 2,4-D-sodium. Examples of suitable esters of 2,4-D are 2,4-D-butotyl, 2,4-D-2-butoxypropyl, 2,4-D-3-butoxypropyl, 2,4-D-butyl, 2,4-D-ethyl, 2,4-D-ethylhexyl, 2,4-D-isobutyl, 2,4-D-isooctyl, 2,4-Disopropyl, 2,4-D-meptyl, 2,4-D-methyl, 2,4-D-octyl, 2,4-D-pentyl, 2,4-D-propyl, 2,4-D-tefuryl. Suitable salts of 2,4-DB are for example 2,4-DB-sodium, 2,4-DB-potassium and 2,4-DB-dimethylammonium. Suitable esters of 2,4-DB are for example 2,4-DB-butyl and 2,4-DB-isoctyl. Suitable salts of dichlorprop are for example dichlorprop-potassium and dichlorprop-dimethylammonium. Examples of suitable esters of dichlorprop are dichlorprop-butotyl and dichlorprop-isoctyl.

Suitable salts and esters of MCPA include MCPA-butotyl, MCPA-butyl, MCPA-dimethylammonium, MCPA-diolamine, MCPA-ethyl, MCPA-thioethyl, MCPA-2-ethylhexyl, MCPA-isobutyl, MCPA-isoctyl, MCPA-isopropyl, MCPA-isopropylammonium, MCPA-methyl, MCPA-olamine, MCPA-potassium, MCPA-sodium and MCPA-trolamine.

A suitable salt of MCPB is MCPB sodium. A suitable ester of MCPB is MCPB-ethyl.

Suitable salts of clopyralid are clopyralid-potassium, clopyralid-olamine and clopyralid-tris-(2-hydroxypropyl)ammonium. Example of suitable esters of clopyralid is clopyralid-methyl.

Examples of a suitable ester of fluroxypyr are fluroxypyr-meptyl and fluroxypyr-2-butoxy-1-methylethyl, wherein fluroxypyr-meptyl is preferred.

Suitable salts of picloram are picloram-dimethylammonium, picloram-potassium, picloram-triisopropanolammonium, picloram-triisopropylammonium and picloram-trolamine. A suitable ester of picloram is picloram-isoctyl.

A suitable salt of triclopyr is triclopyr-triethylammonium. Suitable esters of triclopyr are for example triclopyr-ethyl and triclopyr-butotyl.

Suitable salts and esters of chloramben include chloramben-ammonium, chloramben-diolamine, chloramben-methyl, chloramben-methylammonium and chloramben-sodium. Suitable salts and esters of 2,3,6-TBA include 2,3,6-TBA-dimethylammonium, 2,3,6-TBA-lithium, 2,3,6-TBA-potassium and 2,3,6-TBA-sodium.

Suitable salts and esters of aminopyralid include aminopyralid-potassium and aminopyralid-tris(2-hydroxypropyl)ammonium.

Suitable salts of glyphosate are for example glyphosate-ammonium, glyphosate-diammonium, glyphoste-dimethylammonium, glyphosate-isopropylammonium, glyphosate-potassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is for example glufosinate-ammonium.

A suitable salt of glufosinate-P is for example glufosinate-P-ammonium.

Suitable salts and esters of bromoxynil are for example bromoxynil-butyrate, bromoxynil-heptanoate, bromoxynil-octanoate, bromoxynil-potassium and bromoxynil-sodium.

Suitable salts and esters of ioxonil are for example ioxonil-octanoate, ioxonil-potassium and ioxonil-sodium.

Suitable salts and esters of mecoprop include mecoprop-butotyl, mecoprop-dimethylammonium, mecoprop-diolamine, mecoprop-ethadyl, mecoprop-2-ethylhexyl, mecoprop-isoctyl, mecopropmethyl, mecoprop-potassium, mecoprop-sodium and mecoprop-trolamine.

Suitable salts of mecoprop-P are for example mecoprop-P-butotyl, mecoprop-P-dimethylammonium, mecoprop-P-2-ethylhexyl, mecoprop-P-isobutyl, mecoprop-P-potassium and mecoprop-P-sodium.

A suitable salt of diflufenzopyr is for example diflufenzopyr-sodium.

A suitable salt of naptalam is for example naptalam-sodium.

Suitable salts and esters of aminocyclopyrachlor are for example aminocyclopyrachlor-dimethylammonium, aminocyclopyrachlor-methyl, aminocyclopyrachlor-triisopropanolammonium, aminocyclopyrachlor-sodium and aminocyclopyrachlor-potassium.

A suitable salt of quinclorac is for example quinclorac-dimethylammonium.

A suitable salt of quinmerac is for example quinclorac-dimethylammonium.

A suitable salt of imazamox is for example imazamox-ammonium.

Suitable salts of imazapic are for example imazapic-ammonium and imazapic-isopropylammonium.

Suitable salts of imazapyr are for example imazapyr-ammonium and imazapyr-isopropylammonium.

A suitable salt of imazaquin is for example imazaquin-ammonium.

Suitable salts of imazethapyr are for example imazethapyr-ammonium and imazethapyr-isopropylammonium.

A suitable salt of topramezone is for example topramezone-sodium.

According to a preferred embodiment of the invention, the composition comprises as herbicidal active compound B or component B, at least one, preferably exactly one herbicide B.

According to another preferred embodiment of the invention, the composition comprises as herbicidal active compound B or component B, at least two, preferably exactly two herbicides B different from each other.

According to another preferred embodiment of the invention, the composition comprises as herbicidal active compound B or component B, at least three, preferably exactly three herbicides B different from each other.

According to another preferred embodiment of the invention, the composition comprises as safening component C at least one, preferably exactly one safener C.

According to another preferred embodiment of the invention, the composition comprises as component B, at least one, preferably exactly one herbicide B, and at least one, preferably exactly one, safener C.

According to another preferred embodiment of the invention, the composition comprises as component B, preferably exactly two herbicides B different from each other, and at least one, preferably exactly one, safener C.

According to another preferred embodiment of the invention, the composition comprises as component B, at least three, preferably exactly three herbicides B different from each other, and at least one, preferably exactly one, safener C.

According to another preferred embodiment of the invention, the composition comprises as component A at least one, preferably exactly one compound of formula I, preferably of formula I.1, I.2 or I.3, especially preferred the compound I.a, and as component B, at least one, preferably exactly one, herbicide B.

According to another preferred embodiment of the invention, the composition comprises as component A at least one, preferably exactly one compound of formula I, preferably of formula I.1, I.2 or I.3, especially preferred the compound I.a, and as component B, at least two, preferably exactly two, herbicides B different from each other.

According to another preferred embodiment of the invention, the composition comprises as component A at least one, preferably exactly one compound of formula I, preferably of formula I.1, I.2 or I.3, especially preferred the compound I.a, and as component B, at least three, preferably exactly three herbicides, B different from each other.

According to another preferred embodiment of the invention, the composition comprises as active components at least one, preferably exactly one compound of formula I, preferably of formula I.1, I.2 or I.3, especially preferred the compound I.a, and at least one, preferably exactly one, herbicide B.

According to another preferred embodiment of the invention, the composition comprises as active components at least one, preferably exactly one compound of formula I, preferably of formula I.1, I.2 or I.3, especially preferred the compound I.a, and at least two, preferably exactly two, herbicides B different from each other.

According to another preferred embodiment of the invention, the composition comprises as active components at least one, preferably exactly one compound of formula I, preferably of formula I.1, I.2 or I.3, especially preferred the compound I.a, and at least three, preferably exactly three, herbicides B different from each other.

According to another preferred embodiment of the invention, the composition comprises as active components at least one, preferably exactly one compound of formula I, preferably of formula I.1, I.2 or I.3, especially preferred the compound I.a, and at least one, preferably exactly one, safener C.

According to another preferred embodiment of the invention, the composition comprises as active components at least one, preferably exactly one compound of formula I, preferably of formula I.1, I.2 or I.3, especially preferred the compound I.a, at least one, preferably exactly one, herbicide B, and at least one, preferably exactly one safener C.

According to another preferred embodiment of the invention, the composition comprises as active components at least one, preferably exactly one compound of formula I, preferably of formula I.1, I.2 or I.3, especially preferred the compound I.a, at least two, preferably exactly two herbicides B different from each other, and at least one, preferably exactly one, safener C.

According to another preferred embodiment of the invention, the composition comprises as active components at least one, preferably exactly one compound of formula I, preferably of formula I.1, I.2 or I.3, especially preferred the compound I.a, at least three, preferably exactly three herbicides B different from each other, and at least one, preferably exactly one, safener C.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b1), in particular selected from the group consisting of clodinafop-propargyl, cycloxydim, cyhalofop-butyl, fenoxaprop-P-ethyl, pinoxaden, profoxydim, tepraloxydim, tralkoxydim, esprocarb, prosulfocarb, thiobencarb and triallate.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b2), in particular selected from the group consisting of bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, mesosulfuron, metazosulfuron, nicosulfuron, penoxsulam, propoxycarbazon-sodium, pyrazosulfuron-ethyl, pyroxsulam, rimsulfuron, sulfosulfuron, thiencarbazon-methyl and tritosulfuron.

According to another preferred embodiment of the invention, the composition comprises, in addition to a compound of formula I, especially an active compound from the group consisting I.1, I.2 and I.3, in particular the compound I.a, at least one and especially exactly one herbicidally active compound from group b3), in particular selected from the group consisting of ametryn, atrazine, diuron, fluometuron, hexazinone, isoproturon, linuron, metribuzin, paraquat, paraquat-dichloride, propanil, terbutryn and terbuthylazine.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b4), in particular selected from the group consisting of flumioxazin, oxyfluorfen, saflufenacil, sulfentrazone, ethyl[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione, 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione and 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b5), in particular selected from the group consisting of clomazone, diflufenican, flurochloridone, isoxaflutole, mesotrione, picolinafen, sulcotrione, tefuryltrione, tembotrione, topramezone, bicyclopyrone, amitrole and flumeturon.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b6), in particular selected from the group consisting of glyphosate, glyphosate-isopropylammonium and glyphosate-trimesium (sulfosate).

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b7), in particular selected from the group consisting of glufosinate, glufosinate-P and glufosinate-ammonium.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b9), in particular selected from the group consisting of pendimethalin and trifluralin.

According to another preferred embodiment of the invention, the composition comprises, in addition to a compound of formula I, especially an active compound from the group consisting I.1, I.2 and I.3, in particular the compound I.a, at least one and especially exactly one herbicidally active compound from group b10), in particular selected from the group consisting of acetochlor, cafenstrole, dimethenamid-P, fentrazamide, flufenacet, mefenacet, metazachlor, metolachlor, S-metolachlor, fenoxasulfone and pyroxasulfone. Likewise, preference is given to compositions comprising in addition to a compounds of formula I, especially an active compound from the group consisting of I.1, I.2 and I.3, in particular the compound I.a, at least one and especially exactly one herbicidally active compound from group b10), in particular selected from the group consisting of isoxazoline compounds of the formulae II.1, II.2, II.3, II.4, II.5, II.6, II.7, II.8 and II.9, as defined above.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b11), in particular isoxaben. Likewise, preference is given to compositions comprising in addition to a compound of formula I, especially an active compound from the group consisting of I.1, I.2 and I.3, in particular the compound I.a, at least one and especially exactly one herbicidally active compound from group b11), in particular selected from the group consisting of piperazine compounds of formula Ill as defined above.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b13), in particular selected from the group consisting of 2,4-D and its salts and esters, aminopyralid and its salts such as aminopyralid-tris(2-hydroxypropyl)ammonium and its esters, clopyralid and its salts and esters, dicamba and its salts and esters, fluroxypyr-meptyl, quinclorac, quinmerac and aminocyclopyrachlor and its salts and esters.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b14), in particular selected from the group consisting of diflufenzopyr and diflufenzopyr-sodium.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from group b15), in particular selected from the group consisting of dymron (=daimuron), indanofan, indaziflam, oxaziclomefone and triaziflam.

According to another preferred embodiment of the invention, the composition comprises, in addition to a compound of formula I, especially an active compound from the group consisting of I.1, I.2 and I.3, in particular the compound I.a, at least one and especially exactly one herbicidally active compound from the safeners C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Further preferred embodiments relate to ternary compositions which correspond to the binary compositions mentioned above and additionally comprise a safener C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Here and below, the term “binary compositions” includes compositions comprising one or more, for example 1, 2 or 3, active compounds of the formula I and either one or more, for example 1, 2 or 3, herbicides B or one or more safeners. Correspondingly, the term “ternary compositions” includes compositions comprising one or more, for example 1, 2 or 3, active compounds of the formula I, one or more, for example 1, 2 or 3, herbicides B and one or more, for example 1, 2 or 3, safeners C.

In binary compositions comprising at least one compound of the formula I as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one compound of the formula I as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising both at least one compound of formula I as component A, at least one herbicide B and at least one safener C, the relative proportions by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1, the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1, and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. The weight ratio of components A+B to component C is preferably in the range of from 1:500 to 500:1, in particular in the range of from 1:250 to 250:1 and particularly preferably in the range of from 1:75 to 75:1.

Particularly preferred herbicides B are the herbicides B as defined above; in particular the herbicides B.1-B.187 listed below in table B:

TABLE B

| | Herbicide B |
|---|---|
| B.1 | clethodim |
| B.2 | clodinafop-propargyl |
| B.3 | cycloxydim |
| B.4 | cyhalofop-butyl |
| B.5 | fenoxaprop-ethyl |
| B.6 | fenoxaprop-P-ethyl |
| B.7 | metamifop |
| B.8 | pinoxaden |
| B.9 | profoxydim |
| B.10 | sethoxydim |
| B.11 | tepraloxydim |
| B.12 | tralkoxydim |
| B.13 | esprocarb |
| B.14 | ethofumesate |
| B.15 | molinate |
| B.16 | prosulfocarb |
| B.17 | thiobencarb |
| B.18 | triallate |
| B.19 | bensulfuron-methyl |
| B.20 | bispyribac-sodium |
| B.21 | cloransulam-methyl |
| B.22 | chlorsulfuron |
| B.23 | clorimuron |
| B.24 | cyclosulfamuron |
| B.25 | diclosulam |
| B.26 | florasulam |
| B.27 | flumetsulam |
| B.28 | flupyrsulfuron-methyl-sodium |
| B.29 | foramsulfuron |
| B.30 | imazamox |
| B.31 | imazamox-ammonium |
| B.32 | imazapic |
| B.33 | imazapic-ammonium |
| B.34 | imazapic-isopropylammonium |
| B.35 | imazapyr |
| B.36 | imazapyr-ammonium |
| B.37 | imazapyr-isopropylammonium |
| B.38 | imazaquin |
| B.39 | imazaquin-ammonium |
| B.40 | imazethapyr |
| B.41 | imazethapyr-ammonium |
| B.42 | imazethapyr-isopropylammonium |
| B.43 | imazosulfuron |
| B.44 | iodosulfuron-methyl-sodium |
| B.45 | iofensulfuron |
| B.46 | iofensulfuron-sodium |
| B.47 | mesosulfuron-methyl |
| B.48 | metazosulfuron |
| B.49 | metsulfuron-methyl |
| B.50 | metosulam |
| B.51 | nicosulfuron |
| B.52 | penoxsulam |
| B.53 | propoxycarbazon-sodium |
| B.54 | pyrazosulfuron-ethyl |
| B.55 | pyribenzoxim |
| B.56 | pyriftalid |
| B.57 | pyroxsulam |
| B.58 | propyrisulfuron |
| B.59 | rimsulfuron |
| B.60 | sulfosulfuron |
| B.61 | thiencarbazone-methyl |
| B.62 | thifensulfuron-methyl |
| B.63 | tribenuron-methyl |
| B.64 | tritosulfuron |
| B.65 | triafamone |
| B.66 | ametryne |
| B.67 | atrazine |
| B.68 | bentazon |
| B.69 | bromoxynil |
| B.70 | bromoxynil-octanoate |
| B.71 | bromoxynil-heptanoate |
| B.72 | bromoxynil-potassium |
| B.73 | diuron |
| B.74 | fluometuron |
| B.75 | hexazinone |
| B.76 | isoproturon |
| B.77 | linuron |
| B.78 | metamitron |
| B.79 | metribuzin |
| B.80 | propanil |
| B.81 | simazin |
| B.82 | terbuthylazine |
| B.83 | terbutryn |
| B.84 | paraquat-dichloride |
| B.85 | acifluorfen |
| B.86 | butafenacil |
| B.87 | carfentrazone-ethyl |
| B.88 | flumioxazin |
| B.89 | fomesafen |
| B.90 | oxadiargyl |
| B.91 | oxyfluorfen |
| B.92 | saflufenacil |
| B.93 | sulfentrazone |
| B.94 | ethyl [3-[2-chloro-4-fluoro-5-(1-methyl-6-trifluoromethyl-2,4-di- |
| | oxo-1,2,3,4-tetrahydropyrimidin-3-yl)phenoxy]-2-pyridyl- |
| | oxy]acetate (CAS 353292-31-6) |
| B.95 | 1,5-dimethyl-6-thioxo-3-(2,2,7-trifluoro-3-oxo-4-(prop-2-ynyl)- |
| | 3,4-dihydro-2H-benzo[b][1,4]-oxazin-6-yl)-1,3,5-triazinane- |
| | 2,4-dione |
| B.96 | benzobicyclon |
| B.97 | clomazone |
| B.98 | diflufenican |
| B.99 | flurochloridone |
| B.100 | isoxaflutole |
| B.101 | mesotrione |
| B.102 | norflurazone |
| B.103 | picolinafen |
| B.104 | sulcotrione |
| B.105 | tefuryltrione |
| B.106 | tembotrione |
| B.107 | topramezone |
| B.108 | topramezone-sodium |
| B.109 | bicyclopyrone |
| B.110 | amitrole |
| B.111 | fluometuron |
| B.112 | glyphosate |
| B.113 | glyphosate-ammonium |
| B.114 | glyphosate-dimethylammonium |
| B.115 | glyphosate-isopropylammonium |
| B.116 | glyphosate-trimesium (sulfosate) |
| B.117 | glyphosate-potassium |
| B.118 | glufosinate |
| B.119 | glufosinate-ammonium |
| B.120 | glufosinate-P |
| B.121 | glufosinate-P-ammonium |
| B.122 | pendimethalin |
| B.123 | trifluralin |
| B.124 | acetochlor |
| B.125 | butachlor |
| B.126 | cafenstrole |
| B.127 | dimethenamid-P |
| B.128 | fentrazamide |
| B.129 | flufenacet |
| B.130 | mefenacet |
| B.131 | metazachlor |
| B.132 | metolachlor |
| B.133 | S-metolachlor |
| B.134 | pretilachlor |
| B.135 | fenoxasulfone |
| B.136 | isoxaben |
| B.137 | ipfencarbazone |
| B.138 | pyroxasulfone |
| B.139 | 2,4-D |
| B.140 | 2,4-D-isobutyl |
| B.141 | 2,4-D-dimethylammonium |
| B.142 | 2,4-D-N,N,N-trimethylethanolammonium |
| B.143 | aminopyralid |
| B.144 | aminopyralid-methyl |
| B.145 | aminopyralid-tris(2-hydroxypropyl)ammonium |
| B.146 | clopyralid |
| B.147 | clopyralid-methyl |
| B.148 | clopyralid-olamine |
| B.149 | dicamba |
| B.150 | dicamba-butotyl |
| B.151 | dicamba-diglycolamine |
| B.152 | dicamba-dimethylammonium |
| B.153 | dicamba-diolamine |
| B.154 | dicamba-isopropylammonium |
| B.155 | dicamba-potassium |
| B.156 | dicamba-sodium |
| B.157 | dicamba-trolamine |
| B.158 | dicamba-N,N-bis-(3-aminopropyl)methylamine |
| B.159 | dicamba-diethylenetriamine |
| B.160 | fluroxypyr |
| B.161 | fluroxypyr-meptyl |
| B.162 | MCPA |
| B.163 | MCPA-2-ethylhexyl |
| B.164 | MCPA-dimethylammonium |
| B.165 | quinclorac |
| B.166 | quinclorac-dimethylammonium |
| B.167 | quinmerac |
| B.168 | quinmerac-dimethylammonium |
| B.169 | aminocyclopyrachlor |
| B.170 | aminocyclopyrachlor-potassium |
| B.171 | aminocyclopyrachlor-methyl |
| B.172 | diflufenzopyr |
| B.173 | diflufenzopyr-sodium |
| B.174 | dymron |
| B.175 | indanofan |
| B.176 | indaziflam |
| B.177 | oxaziclomefone |
| B.178 | triaziflam |
| B.179 | II.1 |
| B.180 | II.2 |
| B.181 | II.3 |
| B.182 | II.4 |
| B.183 | II.5 |
| B.184 | II.6 |
| B.185 | II.7 |
| B.186 | II.8 |
| B.187 | II.9 |

Particularly preferred safeners C, which, as component C, are constituent of the composition according to the invention are the safeners C as defined above; in particular the safeners C.1-C.17 listed below in table C:

TABLE C

| | Safener C |
|---|---|
| C.1 | benoxacor |
| C.2 | cloquintocet |
| C.3 | cloquintocet-mexyl |
| C.4 | cyprosulfamide |
| C.5 | dichlormid |
| C.6 | fenchlorazole |
| C.7 | fenchlorazole-ethyl |
| C.8 | fenclorim |
| C.9 | furilazole |
| C.10 | isoxadifen |
| C.11 | isoxadifen-ethyl |
| C.12 | mefenpyr |
| C.13 | mefenpyr-diethyl |
| C.14 | naphtalic acid anhydride |
| C.15 | 4-(dichloroacetyl)-1-oxa-4-azaspiro[4.5]decane |
| | (MON4660, CAS 71526-07-3) |
| C.16 | 2,2,5-trimethyl-3-(dichloroacetyl)-1,3-oxazolidine |
| | (R-29148, CAS 52836-31-4) |
| C.17 | N-(2-Methoxybenzoyl)-4-[(methylaminocarbonyl)ami- |
| | no]benzenesulfonamide (CAS 129531-12-0) |

The weight ratios of the individual components in the preferred mixtures mentioned below are within the limits given above, in particular within the preferred limits.

Particularly preferred are the compositions mentioned below comprising the compounds of formula I as defined and the substance(s) as defined in the respective row of table 1; especially preferred comprising as only herbicidal active compounds the compounds of formula I as defined and the substance(s) as defined in the respective row of table 1; most preferably comprising as only active compounds the compounds of formula I as defined and the substance(s) as defined in the respective row of table 1.

Particularly preferred are compositions 1.1 to 1.3383, comprising the compound I.a and the substance(s) as defined in the respective row of table 1:

TABLE 1
(compositions 1.1 to 1.3383):

| comp. | herbi- | safe- |
|---|---|---|
| no. | cide B | ner C |
| 1.1 | B.1 | — |
| 1.2 | B.2 | — |
| 1.3 | B.3 | — |
| 1.4 | B.4 | — |
| 1.5 | B.5 | — |
| 1.6 | B.6 | — |
| 1.7 | B.7 | — |
| 1.8 | B.8 | — |
| 1.9 | B.9 | — |
| 1.10 | B.10 | — |
| 1.11 | B.11 | — |
| 1.12 | B.12 | — |
| 1.13 | B.13 | — |
| 1.14 | B.14 | — |
| 1.15 | B.15 | — |
| 1.16 | B.16 | — |
| 1.17 | B.17 | — |
| 1.18 | B.18 | — |
| 1.19 | B.19 | — |
| 1.20 | B.20 | — |
| 1.21 | B.21 | — |
| 1.22 | B.22 | — |
| 1.23 | B.23 | — |
| 1.24 | B.24 | — |
| 1.25 | B.25 | — |
| 1.26 | B.26 | — |
| 1.27 | B.27 | — |
| 1.28 | B.28 | — |
| 1.29 | B.29 | — |
| 1.30 | B.30 | — |
| 1.31 | B.31 | — |
| 1.32 | B.32 | — |
| 1.33 | B.33 | — |
| 1.34 | B.34 | — |
| 1.35 | B.35 | — |
| 1.36 | B.36 | — |
| 1.37 | B.37 | — |
| 1.38 | B.38 | — |
| 1.39 | B.39 | — |
| 1.40 | B.40 | — |
| 1.41 | B.41 | — |
| 1.42 | B.42 | — |
| 1.43 | B.43 | — |
| 1.44 | B.44 | — |
| 1.45 | B.45 | — |
| 1.46 | B.46 | — |
| 1.47 | B.47 | — |
| 1.48 | B.48 | — |
| 1.49 | B.49 | — |
| 1.50 | B.50 | — |
| 1.51 | B.51 | — |
| 1.52 | B.52 | — |
| 1.53 | B.53 | — |
| 1.54 | B.54 | — |
| 1.55 | B.55 | — |
| 1.56 | B.56 | — |
| 1.57 | B.57 | — |
| 1.58 | B.58. | — |
| 1.59 | B.59 | — |
| 1.60 | B.60 | — |
| 1.61 | B.61 | — |
| 1.62 | B.62 | — |
| 1.63 | B.63 | — |
| 1.64 | B.64 | — |
| 1.65 | B.65 | — |
| 1.66 | B.66 | — |
| 1.67 | B.67 | — |
| 1.68 | B.68 | — |
| 1.69 | B.69 | — |
| 1.70 | B.70 | — |
| 1.71 | B.71 | — |
| 1.72 | B.72 | — |
| 1.73 | B.73 | — |
| 1.74 | B.74 | — |
| 1.75 | B.75 | — |
| 1.76 | B.76 | — |
| 1.77 | B.77 | — |
| 1.78 | B.78 | — |
| 1.79 | B.79 | — |
| 1.80 | B.80 | — |
| 1.81 | B.81 | — |
| 1.82 | B.82 | — |
| 1.83 | B.83 | — |
| 1.84 | B.84 | — |
| 1.85 | B.85 | — |
| 1.86 | B.86 | — |
| 1.87 | B.87 | — |
| 1.88 | B.88 | — |
| 1.89 | B.89 | — |
| 1.90 | B.90 | — |
| 1.91 | B.91 | — |
| 1.92 | B.92 | — |
| 1.93 | B.93 | — |
| 1.94 | B.94 | — |
| 1.95 | B.95 | — |
| 1.96 | B.96 | — |
| 1.97 | B.97 | — |
| 1.98 | B.98 | — |
| 1.99 | B.99 | — |
| 1.100 | B.100 | — |
| 1.101 | B.101 | — |
| 1.102 | B.102 | — |
| 1.103 | B.103 | — |
| 1.104 | B.104 | — |
| 1.105 | B.105 | — |
| 1.106 | B.106 | — |
| 1.107 | B.107 | — |
| 1.108 | B.108 | — |
| 1.109 | B.109 | — |
| 1.110 | B.110 | — |
| 1.111 | B.111 | — |
| 1.112 | B.112 | — |
| 1.113 | B.113 | — |
| 1.114 | B.114 | — |
| 1.115 | B.115 | — |
| 1.116 | B.116 | — |
| 1.117 | B.117 | — |
| 1.118 | B.118 | — |
| 1.119 | B.119 | — |
| 1.120 | B.120 | — |
| 1.121 | B.121 | — |
| 1.122 | B.122 | — |
| 1.123 | B.123 | — |
| 1.124 | B.124 | — |
| 1.125 | B.125 | — |
| 1.126 | B.126 | — |
| 1.127 | B.127 | — |
| 1.128 | B.128 | — |
| 1.129 | B.129 | — |
| 1.130 | B.130 | — |
| 1.131 | B.131 | — |
| 1.132 | B.132 | — |
| 1.133 | B.133 | — |
| 1.134 | B.134 | — |
| 1.135 | B.135 | — |
| 1.136 | B.136 | — |
| 1.137 | B.137 | — |
| 1.138 | B.138 | — |
| 1.139 | B.139 | — |
| 1.140 | B.140 | — |
| 1.141 | B.141 | — |
| 1.142 | B.142 | — |
| 1.143 | B.143 | — |
| 1.144 | B.144 | — |
| 1.145 | B.145 | — |
| 1.146 | B.146 | — |
| 1.147 | B.147 | — |
| 1.148 | B.148 | — |
| 1.149 | B.149 | — |
| 1.150 | B.150 | — |
| 1.151 | B.151 | — |
| 1.152 | B.152 | — |
| 1.153 | B.153 | — |
| 1.154 | B.154 | — |
| 1.155 | B.155 | — |
| 1.156 | B.156 | — |
| 1.157 | B.157 | — |
| 1.158 | B.158 | — |
| 1.159 | B.159 | — |
| 1.160 | B.160 | — |
| 1.161 | B.161 | — |
| 1.162 | B.162 | — |
| 1.163 | B.163 | — |
| 1.164 | B.164 | — |
| 1.165 | B.165 | — |
| 1.166 | B.166 | — |
| 1.167 | B.167 | — |
| 1.168 | B.168 | — |
| 1.169 | B.169 | — |
| 1.170 | B.170 | — |
| 1.171 | B.171 | — |
| 1.172 | B.172 | — |
| 1.173 | B.173 | — |
| 1.174 | B.174 | — |
| 1.175 | B.175 | — |
| 1.176 | B.176 | — |
| 1.177 | B.177 | — |
| 1.178 | B.178 | — |
| 1.179 | B.179 | — |
| 1.180 | B.180 | — |
| 1.181 | B.181 | — |
| 1.182 | B.182 | — |
| 1.183 | B.183 | — |
| 1.184 | B.184 | — |
| 1.185 | B.185 | — |
| 1.186 | B.186 | — |
| 1.187 | B.187 | — |
| 1.188 | B.1 | C.1 |
| 1.189 | B.2 | C.1 |
| 1.190 | B.3 | C.1 |
| 1.191 | B.4 | C.1 |
| 1.192 | B.5 | C.1 |
| 1.193 | B.6 | C.1 |
| 1.194 | B.7 | C.1 |
| 1.195 | B.8 | C.1 |
| 1.196 | B.9 | C.1 |
| 1.197 | B.10 | C.1 |
| 1.198 | B.11 | C.1 |
| 1.199 | B.12 | C.1 |
| 1.200 | B.13 | C.1 |
| 1.201 | B.14 | C.1 |
| 1.202 | B.15 | C.1 |
| 1.203 | B.16 | C.1 |
| 1.204 | B.17 | C.1 |
| 1.205 | B.18 | C.1 |
| 1.206 | B.19 | C.1 |
| 1.207 | B.20 | C.1 |
| 1.208 | B.21 | C.1 |
| 1.209 | B.22 | C.1 |
| 1.210 | B.23 | C.1 |
| 1.211 | B.24 | C.1 |
| 1.212 | B.25 | C.1 |
| 1.213 | B.26 | C.1 |
| 1.214 | B.27 | C.1 |
| 1.215 | B.28 | C.1 |
| 1.216 | B.29 | C.1 |
| 1.217 | B.30 | C.1 |
| 1.218 | B.31 | C.1 |
| 1.219 | B.32 | C.1 |
| 1.220 | B.33 | C.1 |
| 1.221 | B.34 | C.1 |
| 1.222 | B.35 | C.1 |
| 1.223 | B.36 | C.1 |
| 1.224 | B.37 | C.1 |
| 1.225 | B.38 | C.1 |
| 1.226 | B.39 | C.1 |
| 1.227 | B.40 | C.1 |
| 1.228 | B.41 | C.1 |
| 1.229 | B.42 | C.1 |
| 1.230 | B.43 | C.1 |
| 1.231 | B.44 | C.1 |
| 1.232 | B.45 | C.1 |
| 1.233 | B.46 | C.1 |
| 1.234 | B.47 | C.1 |
| 1.235 | B.48 | C.1 |
| 1.236 | B.49 | C.1 |
| 1.237 | B.50 | C.1 |
| 1.238 | B.51 | C.1 |
| 1.239 | B.52 | C.1 |
| 1.240 | B.53 | C.1 |
| 1.241 | B.54 | C.1 |
| 1.242 | B.55 | C.1 |
| 1.243 | B.56 | C.1 |
| 1.244 | B.57 | C.1 |
| 1.245 | B.58. | C.1 |
| 1.246 | B.59 | C.1 |
| 1.247 | B.60 | C.1 |
| 1.248 | B.61 | C.1 |
| 1.249 | B.62 | C.1 |
| 1.250 | B.63 | C.1 |
| 1.251 | B.64 | C.1 |
| 1.252 | B.65 | C.1 |
| 1.253 | B.66 | C.1 |
| 1.254 | B.67 | C.1 |
| 1.255 | B.68 | C.1 |
| 1.256 | B.69 | C.1 |
| 1.257 | B.70 | C.1 |
| 1.258 | B.71 | C.1 |
| 1.259 | B.72 | C.1 |
| 1.260 | B.73 | C.1 |
| 1.261 | B.74 | C.1 |
| 1.262 | B.75 | C.1 |
| 1.263 | B.76 | C.1 |
| 1.264 | B.77 | C.1 |
| 1.265 | B.78 | C.1 |
| 1.266 | B.79 | C.1 |
| 1.267 | B.80 | C.1 |
| 1.268 | B.81 | C.1 |
| 1.269 | B.82 | C.1 |
| 1.270 | B.83 | C.1 |
| 1.271 | B.84 | C.1 |
| 1.272 | B.85 | C.1 |
| 1.273 | B.86 | C.1 |
| 1.274 | B.87 | C.1 |
| 1.275 | B.88 | C.1 |
| 1.276 | B.89 | C.1 |
| 1.277 | B.90 | C.1 |
| 1.278 | B.91 | C.1 |
| 1.279 | B.92 | C.1 |
| 1.280 | B.93 | C.1 |
| 1.281 | B.94 | C.1 |
| 1.282 | B.95 | C.1 |
| 1.283 | B.96 | C.1 |
| 1.284 | B.97 | C.1 |
| 1.285 | B.98 | C.1 |
| 1.286 | B.99 | C.1 |
| 1.287 | B.100 | C.1 |
| 1.288 | B.101 | C.1 |
| 1.289 | B.102 | C.1 |
| 1.290 | B.103 | C.1 |
| 1.291 | B.104 | C.1 |
| 1.292 | B.105 | C.1 |
| 1.293 | B.106 | C.1 |
| 1.294 | B.107 | C.1 |
| 1.295 | B.108 | C.1 |
| 1.296 | B.109 | C.1 |
| 1.297 | B.110 | C.1 |
| 1.298 | B.111 | C.1 |
| 1.299 | B.112 | C.1 |
| 1.300 | B.113 | C.1 |
| 1.301 | B.114 | C.1 |
| 1.302 | B.115 | C.1 |
| 1.303 | B.116 | C.1 |
| 1.304 | B.117 | C.1 |
| 1.305 | B.118 | C.1 |
| 1.306 | B.119 | C.1 |
| 1.307 | B.120 | C.1 |
| 1.308 | B.121 | C.1 |
| 1.309 | B.122 | C.1 |
| 1.310 | B.123 | C.1 |
| 1.311 | B.124 | C.1 |
| 1.312 | B.125 | C.1 |
| 1.313 | B.126 | C.1 |
| 1.314 | B.127 | C.1 |
| 1.315 | B.128 | C.1 |
| 1.316 | B.129 | C.1 |
| 1.317 | B.130 | C.1 |
| 1.318 | B.131 | C.1 |
| 1.319 | B.132 | C.1 |
| 1.320 | B.133 | C.1 |
| 1.321 | B.134 | C.1 |
| 1.322 | B.135 | C.1 |
| 1.323 | B.136 | C.1 |
| 1.324 | B.137 | C.1 |
| 1.325 | B.138 | C.1 |
| 1.326 | B.139 | C.1 |
| 1.327 | B.140 | C.1 |
| 1.328 | B.141 | C.1 |
| 1.329 | B.142 | C.1 |
| 1.330 | B.143 | C.1 |
| 1.331 | B.144 | C.1 |
| 1.332 | B.145 | C.1 |
| 1.333 | B.146 | C.1 |
| 1.334 | B.147 | C.1 |
| 1.335 | B.148 | C.1 |
| 1.336 | B.149 | C.1 |
| 1.337 | B.150 | C.1 |
| 1.338 | B.151 | C.1 |
| 1.339 | B.152 | C.1 |
| 1.340 | B.153 | C.1 |
| 1.341 | B.154 | C.1 |
| 1.342 | B.155 | C.1 |
| 1.343 | B.156 | C.1 |
| 1.344 | B.157 | C.1 |
| 1.345 | B.158 | C.1 |
| 1.346 | B.159 | C.1 |
| 1.347 | B.160 | C.1 |
| 1.348 | B.161 | C.1 |
| 1.349 | B.162 | C.1 |
| 1.350 | B.163 | C.1 |
| 1.351 | B.164 | C.1 |
| 1.352 | B.165 | C.1 |
| 1.353 | B.166 | C.1 |
| 1.354 | B.167 | C.1 |
| 1.355 | B.168 | C.1 |
| 1.356 | B.169 | C.1 |
| 1.357 | B.170 | C.1 |
| 1.358 | B.171 | C.1 |
| 1.359 | B.172 | C.1 |
| 1.360 | B.173 | C.1 |
| 1.361 | B.174 | C.1 |
| 1.362 | B.175 | C.1 |
| 1.363 | B.176 | C.1 |
| 1.364 | B.177 | C.1 |
| 1.365 | B.178 | C.1 |
| 1.366 | B.179 | C.1 |
| 1.367 | B.180 | C.1 |
| 1.368 | B.181 | C.1 |
| 1.369 | B.182 | C.1 |
| 1.370 | B.183 | C.1 |
| 1.371 | B.184 | C.1 |
| 1.372 | B.185 | C.1 |
| 1.373 | B.186 | C.1 |
| 1.374 | B.187 | C.1 |
| 1.375 | B.1 | C.2 |
| 1.376 | B.2 | C.2 |
| 1.377 | B.3 | C.2 |
| 1.378 | B.4 | C.2 |
| 1.379 | B.5 | C.2 |
| 1.380 | B.6 | C.2 |
| 1.381 | B.7 | C.2 |
| 1.382 | B.8 | C.2 |
| 1.383 | B.9 | C.2 |
| 1.384 | B.10 | C.2 |
| 1.385 | B.11 | C.2 |
| 1.386 | B.12 | C.2 |
| 1.387 | B.13 | C.2 |
| 1.388 | B.14 | C.2 |
| 1.389 | B.15 | C.2 |
| 1.390 | B.16 | C.2 |
| 1.391 | B.17 | C.2 |
| 1.392 | B.18 | C.2 |
| 1.393 | B.19 | C.2 |
| 1.394 | B.20 | C.2 |
| 1.395 | B.21 | C.2 |
| 1.396 | B.22 | C.2 |
| 1.397 | B.23 | C.2 |
| 1.398 | B.24 | C.2 |
| 1.399 | B.25 | C.2 |
| 1.400 | B.26 | C.2 |
| 1.401 | B.27 | C.2 |
| 1.402 | B.28 | C.2 |
| 1.403 | B.29 | C.2 |
| 1.404 | B.30 | C.2 |
| 1.405 | B.31 | C.2 |
| 1.406 | B.32 | C.2 |
| 1.407 | B.33 | C.2 |
| 1.408 | B.34 | C.2 |
| 1.409 | B.35 | C.2 |
| 1.410 | B.36 | C.2 |
| 1.411 | B.37 | C.2 |
| 1.412 | B.38 | C.2 |
| 1.413 | B.39 | C.2 |
| 1.414 | B.40 | C.2 |
| 1.415 | B.41 | C.2 |
| 1.416 | B.42 | C.2 |
| 1.417 | B.43 | C.2 |
| 1.418 | B.44 | C.2 |
| 1.419 | B.45 | C.2 |
| 1.420 | B.46 | C.2 |
| 1.421 | B.47 | C.2 |
| 1.422 | B.48 | C.2 |
| 1.423 | B.49 | C.2 |
| 1.424 | B.50 | C.2 |
| 1.425 | B.51 | C.2 |
| 1.426 | B.52 | C.2 |
| 1.427 | B.53 | C.2 |
| 1.428 | B.54 | C.2 |
| 1.429 | B.55 | C.2 |
| 1.430 | B.56 | C.2 |
| 1.431 | B.57 | C.2 |
| 1.432 | B.58. | C.2 |
| 1.433 | B.59 | C.2 |
| 1.434 | B.60 | C.2 |
| 1.435 | B.61 | C.2 |
| 1.436 | B.62 | C.2 |
| 1.437 | B.63 | C.2 |
| 1.438 | B.64 | C.2 |
| 1.439 | B.65 | C.2 |
| 1.440 | B.66 | C.2 |
| 1.441 | B.67 | C.2 |
| 1.442 | B.68 | C.2 |
| 1.443 | B.69 | C.2 |
| 1.444 | B.70 | C.2 |
| 1.445 | B.71 | C.2 |
| 1.446 | B.72 | C.2 |
| 1.447 | B.73 | C.2 |
| 1.448 | B.74 | C.2 |
| 1.449 | B.75 | C.2 |
| 1.450 | B.76 | C.2 |
| 1.451 | B.77 | C.2 |
| 1.452 | B.78 | C.2 |
| 1.453 | B.79 | C.2 |
| 1.454 | B.80 | C.2 |
| 1.455 | B.81 | C.2 |
| 1.456 | B.82 | C.2 |
| 1.457 | B.83 | C.2 |
| 1.458 | B.84 | C.2 |
| 1.459 | B.85 | C.2 |
| 1.460 | B.86 | C.2 |
| 1.461 | B.87 | C.2 |
| 1.462 | B.88 | C.2 |
| 1.463 | B.89 | C.2 |
| 1.464 | B.90 | C.2 |
| 1.465 | B.91 | C.2 |
| 1.466 | B.92 | C.2 |
| 1.467 | B.93 | C.2 |
| 1.468 | B.94 | C.2 |
| 1.469 | B.95 | C.2 |
| 1.470 | B.96 | C.2 |
| 1.471 | B.97 | C.2 |
| 1.472 | B.98 | C.2 |
| 1.473 | B.99 | C.2 |
| 1.474 | B.100 | C.2 |
| 1.475 | B.101 | C.2 |
| 1.476 | B.102 | C.2 |
| 1.477 | B.103 | C.2 |
| 1.478 | B.104 | C.2 |
| 1.479 | B.105 | C.2 |
| 1.480 | B.106 | C.2 |
| 1.481 | B.107 | C.2 |
| 1.482 | B.108 | C.2 |
| 1.483 | B.109 | C.2 |
| 1.484 | B.110 | C.2 |
| 1.485 | B.111 | C.2 |
| 1.486 | B.112 | C.2 |
| 1.487 | B.113 | C.2 |
| 1.488 | B.114 | C.2 |
| 1.489 | B.115 | C.2 |
| 1.490 | B.116 | C.2 |
| 1.491 | B.117 | C.2 |
| 1.492 | B.118 | C.2 |
| 1.493 | B.119 | C.2 |
| 1.494 | B.120 | C.2 |
| 1.495 | B.121 | C.2 |
| 1.496 | B.122 | C.2 |
| 1.497 | B.123 | C.2 |
| 1.498 | B.124 | C.2 |
| 1.499 | B.125 | C.2 |
| 1.500 | B.126 | C.2 |
| 1.501 | B.127 | C.2 |
| 1.502 | B.128 | C.2 |
| 1.503 | B.129 | C.2 |
| 1.504 | B.130 | C.2 |
| 1.505 | B.131 | C.2 |
| 1.506 | B.132 | C.2 |
| 1.507 | B.133 | C.2 |
| 1.508 | B.134 | C.2 |
| 1.509 | B.135 | C.2 |
| 1.510 | B.136 | C.2 |
| 1.511 | B.137 | C.2 |
| 1.512 | B.138 | C.2 |
| 1.513 | B.139 | C.2 |
| 1.514 | B.140 | C.2 |
| 1.515 | B.141 | C.2 |
| 1.516 | B.142 | C.2 |
| 1.517 | B.143 | C.2 |
| 1.518 | B.144 | C.2 |
| 1.519 | B.145 | C.2 |
| 1.520 | B.146 | C.2 |
| 1.521 | B.147 | C.2 |
| 1.522 | B.148 | C.2 |
| 1.523 | B.149 | C.2 |
| 1.524 | B.150 | C.2 |
| 1.525 | B.151 | C.2 |
| 1.526 | B.152 | C.2 |
| 1.527 | B.153 | C.2 |
| 1.528 | B.154 | C.2 |
| 1.529 | B.155 | C.2 |
| 1.530 | B.156 | C.2 |
| 1.531 | B.157 | C.2 |
| 1.532 | B.158 | C.2 |
| 1.533 | B.159 | C.2 |
| 1.534 | B.160 | C.2 |
| 1.535 | B.161 | C.2 |
| 1.536 | B.162 | C.2 |
| 1.537 | B.163 | C.2 |
| 1.538 | B.164 | C.2 |
| 1.539 | B.165 | C.2 |
| 1.540 | B.166 | C.2 |
| 1.541 | B.167 | C.2 |
| 1.542 | B.168 | C.2 |
| 1.543 | B.169 | C.2 |
| 1.544 | B.170 | C.2 |
| 1.545 | B.171 | C.2 |
| 1.546 | B.172 | C.2 |
| 1.547 | B.173 | C.2 |
| 1.548 | B.174 | C.2 |
| 1.549 | B.175 | C.2 |
| 1.550 | B.176 | C.2 |
| 1.551 | B.177 | C.2 |
| 1.552 | B.178 | C.2 |
| 1.553 | B.179 | C.2 |
| 1.554 | B.180 | C.2 |
| 1.555 | B.181 | C.2 |
| 1.556 | B.182 | C.2 |
| 1.557 | B.183 | C.2 |
| 1.558 | B.184 | C.2 |
| 1.559 | B.185 | C.2 |
| 1.560 | B.186 | C.2 |
| 1.561 | B.187 | C.2 |
| 1.562 | B.1 | C.3 |
| 1.563 | B.2 | C.3 |
| 1.564 | B.3 | C.3 |
| 1.565 | B.4 | C.3 |
| 1.566 | B.5 | C.3 |
| 1.567 | B.6 | C.3 |
| 1.568 | B.7 | C.3 |
| 1.569 | B.8 | C.3 |
| 1.570 | B.9 | C.3 |
| 1.571 | B.10 | C.3 |
| 1.572 | B.11 | C.3 |
| 1.573 | B.12 | C.3 |
| 1.574 | B.13 | C.3 |
| 1.575 | B.14 | C.3 |
| 1.576 | B.15 | C.3 |
| 1.577 | B.16 | C.3 |
| 1.578 | B.17 | C.3 |
| 1.579 | B.18 | C.3 |
| 1.580 | B.19 | C.3 |
| 1.581 | B.20 | C.3 |
| 1.582 | B.21 | C.3 |
| 1.583 | B.22 | C.3 |
| 1.584 | B.23 | C.3 |
| 1.585 | B.24 | C.3 |
| 1.586 | B.25 | C.3 |
| 1.587 | B.26 | C.3 |
| 1.588 | B.27 | C.3 |
| 1.589 | B.28 | C.3 |
| 1.590 | B.29 | C.3 |
| 1.591 | B.30 | C.3 |
| 1.592 | B.31 | C.3 |
| 1.593 | B.32 | C.3 |
| 1.594 | B.33 | C.3 |
| 1.595 | B.34 | C.3 |
| 1.596 | B.35 | C.3 |
| 1.597 | B.36 | C.3 |
| 1.598 | B.37 | C.3 |
| 1.599 | B.38 | C.3 |
| 1.600 | B.39 | C.3 |
| 1.601 | B.40 | C.3 |
| 1.602 | B.41 | C.3 |
| 1.603 | B.42 | C.3 |
| 1.604 | B.43 | C.3 |
| 1.605 | B.44 | C.3 |
| 1.606 | B.45 | C.3 |
| 1.607 | B.46 | C.3 |
| 1.608 | B.47 | C.3 |
| 1.609 | B.48 | C.3 |
| 1.610 | B.49 | C.3 |
| 1.611 | B.50 | C.3 |
| 1.612 | B.51 | C.3 |
| 1.613 | B.52 | C.3 |
| 1.614 | B.53 | C.3 |
| 1.615 | B.54 | C.3 |
| 1.616 | B.55 | C.3 |
| 1.617 | B.56 | C.3 |
| 1.618 | B.57 | C.3 |
| 1.619 | B.58. | C.3 |
| 1.620 | B.59 | C.3 |
| 1.621 | B.60 | C.3 |
| 1.622 | B.61 | C.3 |
| 1.623 | B.62 | C.3 |
| 1.624 | B.63 | C.3 |
| 1.625 | B.64 | C.3 |
| 1.626 | B.65 | C.3 |
| 1.627 | B.66 | C.3 |
| 1.628 | B.67 | C.3 |
| 1.629 | B.68 | C.3 |
| 1.630 | B.69 | C.3 |
| 1.631 | B.70 | C.3 |
| 1.632 | B.71 | C.3 |
| 1.633 | B.72 | C.3 |
| 1.634 | B.73 | C.3 |
| 1.635 | B.74 | C.3 |
| 1.636 | B.75 | C.3 |
| 1.637 | B.76 | C.3 |
| 1.638 | B.77 | C.3 |
| 1.639 | B.78 | C.3 |
| 1.640 | B.79 | C.3 |
| 1.641 | B.80 | C.3 |
| 1.642 | B.81 | C.3 |
| 1.643 | B.82 | C.3 |
| 1.644 | B.83 | C.3 |
| 1.645 | B.84 | C.3 |
| 1.646 | B.85 | C.3 |
| 1.647 | B.86 | C.3 |
| 1.648 | B.87 | C.3 |
| 1.649 | B.88 | C.3 |
| 1.650 | B.89 | C.3 |
| 1.651 | B.90 | C.3 |
| 1.652 | B.91 | C.3 |
| 1.653 | B.92 | C.3 |
| 1.654 | B.93 | C.3 |
| 1.655 | B.94 | C.3 |
| 1.656 | B.95 | C.3 |
| 1.657 | B.96 | C.3 |
| 1.658 | B.97 | C.3 |
| 1.659 | B.98 | C.3 |
| 1.660 | B.99 | C.3 |
| 1.661 | B.100 | C.3 |
| 1.662 | B.101 | C.3 |
| 1.663 | B.102 | C.3 |
| 1.664 | B.103 | C.3 |
| 1.665 | B.104 | C.3 |
| 1.666 | B.105 | C.3 |
| 1.667 | B.106 | C.3 |
| 1.668 | B.107 | C.3 |
| 1.669 | B.108 | C.3 |
| 1.670 | B.109 | C.3 |
| 1.671 | B.110 | C.3 |
| 1.672 | B.111 | C.3 |
| 1.673 | B.112 | C.3 |
| 1.674 | B.113 | C.3 |
| 1.675 | B.114 | C.3 |
| 1.676 | B.115 | C.3 |
| 1.677 | B.116 | C.3 |
| 1.678 | B.117 | C.3 |
| 1.679 | B.118 | C.3 |
| 1.680 | B.119 | C.3 |
| 1.681 | B.120 | C.3 |
| 1.682 | B.121 | C.3 |
| 1.683 | B.122 | C.3 |
| 1.684 | B.123 | C.3 |
| 1.685 | B.124 | C.3 |
| 1.686 | B.125 | C.3 |
| 1.687 | B.126 | C.3 |
| 1.688 | B.127 | C.3 |
| 1.689 | B.128 | C.3 |
| 1.690 | B.129 | C.3 |
| 1.691 | B.130 | C.3 |
| 1.692 | B.131 | C.3 |
| 1.693 | B.132 | C.3 |
| 1.694 | B.133 | C.3 |
| 1.695 | B.134 | C.3 |
| 1.696 | B.135 | C.3 |
| 1.697 | B.136 | C.3 |
| 1.698 | B.137 | C.3 |
| 1.699 | B.138 | C.3 |
| 1.700 | B.139 | C.3 |
| 1.701 | B.140 | C.3 |
| 1.702 | B.141 | C.3 |
| 1.703 | B.142 | C.3 |
| 1.704 | B.143 | C.3 |
| 1.705 | B.144 | C.3 |
| 1.706 | B.145 | C.3 |
| 1.707 | B.146 | C.3 |
| 1.708 | B.147 | C.3 |
| 1.709 | B.148 | C.3 |
| 1.710 | B.149 | C.3 |
| 1.711 | B.150 | C.3 |
| 1.712 | B.151 | C.3 |
| 1.713 | B.152 | C.3 |
| 1.714 | B.153 | C.3 |
| 1.715 | B.154 | C.3 |
| 1.716 | B.155 | C.3 |
| 1.717 | B.156 | C.3 |
| 1.718 | B.157 | C.3 |
| 1.719 | B.158 | C.3 |
| 1.720 | B.159 | C.3 |
| 1.721 | B.160 | C.3 |
| 1.722 | B.161 | C.3 |
| 1.723 | B.162 | C.3 |
| 1.724 | B.163 | C.3 |
| 1.725 | B.164 | C.3 |
| 1.726 | B.165 | C.3 |
| 1.727 | B.166 | C.3 |
| 1.728 | B.167 | C.3 |
| 1.729 | B.168 | C.3 |
| 1.730 | B.169 | C.3 |
| 1.731 | B.170 | C.3 |
| 1.732 | B.171 | C.3 |
| 1.733 | B.172 | C.3 |
| 1.734 | B.173 | C.3 |
| 1.735 | B.174 | C.3 |
| 1.736 | B.175 | C.3 |
| 1.737 | B.176 | C.3 |
| 1.738 | B.177 | C.3 |
| 1.739 | B.178 | C.3 |
| 1.740 | B.179 | C.3 |
| 1.741 | B.180 | C.3 |
| 1.742 | B.181 | C.3 |
| 1.743 | B.182 | C.3 |
| 1.744 | B.183 | C.3 |
| 1.745 | B.184 | C.3 |
| 1.746 | B.185 | C.3 |
| 1.747 | B.186 | C.3 |
| 1.748 | B.187 | C.3 |
| 1.749 | B.1 | C.4 |
| 1.750 | B.2 | C.4 |
| 1.751 | B.3 | C.4 |
| 1.752 | B.4 | C.4 |
| 1.753 | B.5 | C.4 |
| 1.754 | B.6 | C.4 |
| 1.755 | B.7 | C.4 |
| 1.756 | B.8 | C.4 |
| 1.757 | B.9 | C.4 |
| 1.758 | B.10 | C.4 |
| 1.759 | B.11 | C.4 |
| 1.760 | B.12 | C.4 |
| 1.761 | B.13 | C.4 |
| 1.762 | B.14 | C.4 |
| 1.763 | B.15 | C.4 |
| 1.764 | B.16 | C.4 |
| 1.765 | B.17 | C.4 |
| 1.766 | B.18 | C.4 |
| 1.767 | B.19 | C.4 |
| 1.768 | B.20 | C.4 |
| 1.769 | B.21 | C.4 |
| 1.770 | B.22 | C.4 |
| 1.771 | B.23 | C.4 |
| 1.772 | B.24 | C.4 |
| 1.773 | B.25 | C.4 |
| 1.774 | B.26 | C.4 |
| 1.775 | B.27 | C.4 |
| 1.776 | B.28 | C.4 |
| 1.777 | B.29 | C.4 |
| 1.778 | B.30 | C.4 |
| 1.779 | B.31 | C.4 |
| 1.780 | B.32 | C.4 |
| 1.781 | B.33 | C.4 |
| 1.782 | B.34 | C.4 |
| 1.783 | B.35 | C.4 |
| 1.784 | B.36 | C.4 |
| 1.785 | B.37 | C.4 |
| 1.786 | B.38 | C.4 |
| 1.787 | B.39 | C.4 |
| 1.788 | B.40 | C.4 |
| 1.789 | B.41 | C.4 |
| 1.790 | B.42 | C.4 |
| 1.791 | B.43 | C.4 |
| 1.792 | B.44 | C.4 |
| 1.793 | B.45 | C.4 |
| 1.794 | B.46 | C.4 |
| 1.795 | B.47 | C.4 |
| 1.796 | B.48 | C.4 |
| 1.797 | B.49 | C.4 |
| 1.798 | B.50 | C.4 |
| 1.799 | B.51 | C.4 |
| 1.800 | B.52 | C.4 |
| 1.801 | B.53 | C.4 |
| 1.802 | B.54 | C.4 |
| 1.803 | B.55 | C.4 |
| 1.804 | B.56 | C.4 |
| 1.805 | B.57 | C.4 |
| 1.806 | B.58. | C.4 |
| 1.807 | B.59 | C.4 |
| 1.808 | B.60 | C.4 |
| 1.809 | B.61 | C.4 |
| 1.810 | B.62 | C.4 |
| 1.811 | B.63 | C.4 |
| 1.812 | B.64 | C.4 |
| 1.813 | B.65 | C.4 |
| 1.814 | B.66 | C.4 |
| 1.815 | B.67 | C.4 |
| 1.816 | B.68 | C.4 |
| 1.817 | B.69 | C.4 |
| 1.818 | B.70 | C.4 |
| 1.819 | B.71 | C.4 |
| 1.820 | B.72 | C.4 |
| 1.821 | B.73 | C.4 |
| 1.822 | B.74 | C.4 |
| 1.823 | B.75 | C.4 |
| 1.824 | B.76 | C.4 |
| 1.825 | B.77 | C.4 |
| 1.826 | B.78 | C.4 |
| 1.827 | B.79 | C.4 |
| 1.828 | B.80 | C.4 |
| 1.829 | B.81 | C.4 |
| 1.830 | B.82 | C.4 |
| 1.831 | B.83 | C.4 |
| 1.832 | B.84 | C.4 |
| 1.833 | B.85 | C.4 |
| 1.834 | B.86 | C.4 |
| 1.835 | B.87 | C.4 |
| 1.836 | B.88 | C.4 |
| 1.837 | B.89 | C.4 |
| 1.838 | B.90 | C.4 |
| 1.839 | B.91 | C.4 |
| 1.840 | B.92 | C.4 |
| 1.841 | B.93 | C.4 |
| 1.842 | B.94 | C.4 |
| 1.843 | B.95 | C.4 |
| 1.844 | B.96 | C.4 |
| 1.845 | B.97 | C.4 |
| 1.846 | B.98 | C.4 |
| 1.847 | B.99 | C.4 |
| 1.848 | B.100 | C.4 |
| 1.849 | B.101 | C.4 |
| 1.850 | B.102 | C.4 |
| 1.851 | B.103 | C.4 |
| 1.852 | B.104 | C.4 |
| 1.853 | B.105 | C.4 |
| 1.854 | B.106 | C.4 |
| 1.855 | B.107 | C.4 |
| 1.856 | B.108 | C.4 |
| 1.857 | B.109 | C.4 |
| 1.858 | B.110 | C.4 |
| 1.859 | B.111 | C.4 |
| 1.860 | B.112 | C.4 |
| 1.861 | B.113 | C.4 |
| 1.862 | B.114 | C.4 |
| 1.863 | B.115 | C.4 |
| 1.864 | B.116 | C.4 |
| 1.865 | B.117 | C.4 |
| 1.866 | B.118 | C.4 |
| 1.867 | B.119 | C.4 |
| 1.868 | B.120 | C.4 |
| 1.869 | B.121 | C.4 |
| 1.870 | B.122 | C.4 |
| 1.871 | B.123 | C.4 |
| 1.872 | B.124 | C.4 |
| 1.873 | B.125 | C.4 |
| 1.874 | B.126 | C.4 |
| 1.875 | B.127 | C.4 |
| 1.876 | B.128 | C.4 |
| 1.877 | B.129 | C.4 |
| 1.878 | B.130 | C.4 |
| 1.879 | B.131 | C.4 |
| 1.880 | B.132 | C.4 |
| 1.881 | B.133 | C.4 |
| 1.882 | B.134 | C.4 |
| 1.883 | B.135 | C.4 |
| 1.884 | B.136 | C.4 |
| 1.885 | B.137 | C.4 |
| 1.886 | B.138 | C.4 |
| 1.887 | B.139 | C.4 |
| 1.888 | B.140 | C.4 |
| 1.889 | B.141 | C.4 |
| 1.890 | B.142 | C.4 |
| 1.891 | B.143 | C.4 |
| 1.892 | B.144 | C.4 |
| 1.893 | B.145 | C.4 |
| 1.894 | B.146 | C.4 |
| 1.895 | B.147 | C.4 |
| 1.896 | B.148 | C.4 |
| 1.897 | B.149 | C.4 |
| 1.898 | B.150 | C.4 |
| 1.899 | B.151 | C.4 |
| 1.900 | B.152 | C.4 |
| 1.901 | B.153 | C.4 |
| 1.902 | B.154 | C.4 |
| 1.903 | B.155 | C.4 |
| 1.904 | B.156 | C.4 |
| 1.905 | B.157 | C.4 |
| 1.906 | B.158 | C.4 |
| 1.907 | B.159 | C.4 |
| 1.908 | B.160 | C.4 |
| 1.909 | B.161 | C.4 |
| 1.910 | B.162 | C.4 |
| 1.911 | B.163 | C.4 |
| 1.912 | B.164 | C.4 |
| 1.913 | B.165 | C.4 |
| 1.914 | B.166 | C.4 |
| 1.915 | B.167 | C.4 |
| 1.916 | B.168 | C.4 |
| 1.917 | B.169 | C.4 |
| 1.918 | B.170 | C.4 |
| 1.919 | B.171 | C.4 |
| 1.920 | B.172 | C.4 |
| 1.921 | B.173 | C.4 |
| 1.922 | B.174 | C.4 |
| 1.923 | B.175 | C.4 |
| 1.924 | B.176 | C.4 |
| 1.925 | B.177 | C.4 |
| 1.926 | B.178 | C.4 |
| 1.927 | B.179 | C.4 |
| 1.928 | B.180 | C.4 |
| 1.929 | B.181 | C.4 |
| 1.930 | B.182 | C.4 |
| 1.931 | B.183 | C.4 |
| 1.932 | B.184 | C.4 |
| 1.933 | B.185 | C.4 |
| 1.934 | B.186 | C.4 |
| 1.935 | B.187 | C.4 |
| 1.936 | B.1 | C.5 |
| 1.937 | B.2 | C.5 |
| 1.938 | B.3 | C.5 |
| 1.939 | B.4 | C.5 |
| 1.940 | B.5 | C.5 |
| 1.941 | B.6 | C.5 |
| 1.942 | B.7 | C.5 |
| 1.943 | B.8 | C.5 |
| 1.944 | B.9 | C.5 |
| 1.945 | B.10 | C.5 |
| 1.946 | B.11 | C.5 |
| 1.947 | B.12 | C.5 |
| 1.948 | B.13 | C.5 |
| 1.949 | B.14 | C.5 |
| 1.950 | B.15 | C.5 |
| 1.951 | B.16 | C.5 |
| 1.952 | B.17 | C.5 |
| 1.953 | B.18 | C.5 |
| 1.954 | B.19 | C.5 |
| 1.955 | B.20 | C.5 |
| 1.956 | B.21 | C.5 |
| 1.957 | B.22 | C.5 |
| 1.958 | B.23 | C.5 |
| 1.959 | B.24 | C.5 |
| 1.960 | B.25 | C.5 |
| 1.961 | B.26 | C.5 |
| 1.962 | B.27 | C.5 |
| 1.963 | B.28 | C.5 |
| 1.964 | B.29 | C.5 |
| 1.965 | B.30 | C.5 |
| 1.966 | B.31 | C.5 |
| 1.967 | B.32 | C.5 |
| 1.968 | B.33 | C.5 |
| 1.969 | B.34 | C.5 |
| 1.970 | B.35 | C.5 |
| 1.971 | B.36 | C.5 |
| 1.972 | B.37 | C.5 |
| 1.973 | B.38 | C.5 |
| 1.974 | B.39 | C.5 |
| 1.975 | B.40 | C.5 |
| 1.976 | B.41 | C.5 |
| 1.977 | B.42 | C.5 |
| 1.978 | B.43 | C.5 |
| 1.979 | B.44 | C.5 |
| 1.980 | B.45 | C.5 |
| 1.981 | B.46 | C.5 |
| 1.982 | B.47 | C.5 |
| 1.983 | B.48 | C.5 |
| 1.984 | B.49 | C.5 |
| 1.985 | B.50 | C.5 |
| 1.986 | B.51 | C.5 |
| 1.987 | B.52 | C.5 |
| 1.988 | B.53 | C.5 |
| 1.989 | B.54 | C.5 |
| 1.990 | B.55 | C.5 |
| 1.991 | B.56 | C.5 |
| 1.992 | B.57 | C.5 |
| 1.993 | B.58. | C.5 |
| 1.994 | B.59 | C.5 |
| 1.995 | B.60 | C.5 |
| 1.996 | B.61 | C.5 |
| 1.997 | B.62 | C.5 |
| 1.998 | B.63 | C.5 |
| 1.999 | B.64 | C.5 |
| 1.1000 | B.65 | C.5 |
| 1.1001 | B.66 | C.5 |
| 1.1002 | B.67 | C.5 |
| 1.1003 | B.68 | C.5 |
| 1.1004 | B.69 | C.5 |
| 1.1005 | B.70 | C.5 |
| 1.1006 | B.71 | C.5 |
| 1.1007 | B.72 | C.5 |
| 1.1008 | B.73 | C.5 |
| 1.1009 | B.74 | C.5 |
| 1.1010 | B.75 | C.5 |
| 1.1011 | B.76 | C.5 |
| 1.1012 | B.77 | C.5 |
| 1.1013 | B.78 | C.5 |
| 1.1014 | B.79 | C.5 |
| 1.1015 | B.80 | C.5 |
| 1.1016 | B.81 | C.5 |
| 1.1017 | B.82 | C.5 |
| 1.1018 | B.83 | C.5 |
| 1.1019 | B.84 | C.5 |
| 1.1020 | B.85 | C.5 |
| 1.1021 | B.86 | C.5 |
| 1.1022 | B.87 | C.5 |
| 1.1023 | B.88 | C.5 |
| 1.1024 | B.89 | C.5 |
| 1.1025 | B.90 | C.5 |
| 1.1026 | B.91 | C.5 |
| 1.1027 | B.92 | C.5 |
| 1.1028 | B.93 | C.5 |
| 1.1029 | B.94 | C.5 |
| 1.1030 | B.95 | C.5 |
| 1.1031 | B.96 | C.5 |
| 1.1032 | B.97 | C.5 |
| 1.1033 | B.98 | C.5 |
| 1.1034 | B.99 | C.5 |
| 1.1035 | B.100 | C.5 |
| 1.1036 | B.101 | C.5 |
| 1.1037 | B.102 | C.5 |
| 1.1038 | B.103 | C.5 |
| 1.1039 | B.104 | C.5 |
| 1.1040 | B.105 | C.5 |
| 1.1041 | B.106 | C.5 |
| 1.1042 | B.107 | C.5 |
| 1.1043 | B.108 | C.5 |
| 1.1044 | B.109 | C.5 |
| 1.1045 | B.110 | C.5 |
| 1.1046 | B.111 | C.5 |
| 1.1047 | B.112 | C.5 |
| 1.1048 | B.113 | C.5 |
| 1.1049 | B.114 | C.5 |
| 1.1050 | B.115 | C.5 |
| 1.1051 | B.116 | C.5 |
| 1.1052 | B.117 | C.5 |
| 1.1053 | B.118 | C.5 |
| 1.1054 | B.119 | C.5 |
| 1.1055 | B.120 | C.5 |
| 1.1056 | B.121 | C.5 |
| 1.1057 | B.122 | C.5 |
| 1.1058 | B.123 | C.5 |
| 1.1059 | B.124 | C.5 |
| 1.1060 | B.125 | C.5 |
| 1.1061 | B.126 | C.5 |
| 1.1062 | B.127 | C.5 |
| 1.1063 | B.128 | C.5 |
| 1.1064 | B.129 | C.5 |
| 1.1065 | B.130 | C.5 |
| 1.1066 | B.131 | C.5 |
| 1.1067 | B.132 | C.5 |
| 1.1068 | B.133 | C.5 |
| 1.1069 | B.134 | C.5 |
| 1.1070 | B.135 | C.5 |
| 1.1071 | B.136 | C.5 |
| 1.1072 | B.137 | C.5 |
| 1.1073 | B.138 | C.5 |
| 1.1074 | B.139 | C.5 |
| 1.1075 | B.140 | C.5 |
| 1.1076 | B.141 | C.5 |
| 1.1077 | B.142 | C.5 |
| 1.1078 | B.143 | C.5 |
| 1.1079 | B.144 | C.5 |
| 1.1080 | B.145 | C.5 |
| 1.1081 | B.146 | C.5 |
| 1.1082 | B.147 | C.5 |
| 1.1083 | B.148 | C.5 |
| 1.1084 | B.149 | C.5 |
| 1.1085 | B.150 | C.5 |
| 1.1086 | B.151 | C.5 |
| 1.1087 | B.152 | C.5 |
| 1.1088 | B.153 | C.5 |
| 1.1089 | B.154 | C.5 |
| 1.1090 | B.155 | C.5 |
| 1.1091 | B.156 | C.5 |
| 1.1092 | B.157 | C.5 |
| 1.1093 | B.158 | C.5 |
| 1.1094 | B.159 | C.5 |
| 1.1095 | B.160 | C.5 |
| 1.1096 | B.161 | C.5 |
| 1.1097 | B.162 | C.5 |
| 1.1098 | B.163 | C.5 |
| 1.1099 | B.164 | C.5 |
| 1.1100 | B.165 | C.5 |
| 1.1101 | B.166 | C.5 |
| 1.1102 | B.167 | C.5 |
| 1.1103 | B.168 | C.5 |
| 1.1104 | B.169 | C.5 |
| 1.1105 | B.170 | C.5 |
| 1.1106 | B.171 | C.5 |
| 1.1107 | B.172 | C.5 |
| 1.1108 | B.173 | C.5 |
| 1.1109 | B.174 | C.5 |
| 1.1110 | B.175 | C.5 |
| 1.1111 | B.176 | C.5 |
| 1.1112 | B.177 | C.5 |
| 1.1113 | B.178 | C.5 |
| 1.1114 | B.179 | C.5 |
| 1.1115 | B.180 | C.5 |
| 1.1116 | B.181 | C.5 |
| 1.1117 | B.182 | C.5 |
| 1.1118 | B.183 | C.5 |
| 1.1119 | B.184 | C.5 |
| 1.1120 | B.185 | C.5 |
| 1.1121 | B.186 | C.5 |
| 1.1122 | B.187 | C.5 |
| 1.1123 | B.1 | C.6 |
| 1.1124 | B.2 | C.6 |
| 1.1125 | B.3 | C.6 |
| 1.1126 | B.4 | C.6 |
| 1.1127 | B.5 | C.6 |
| 1.1128 | B.6 | C.6 |
| 1.1129 | B.7 | C.6 |
| 1.1130 | B.8 | C.6 |
| 1.1131 | B.9 | C.6 |
| 1.1132 | B.10 | C.6 |
| 1.1133 | B.11 | C.6 |
| 1.1134 | B.12 | C.6 |
| 1.1135 | B.13 | C.6 |
| 1.1136 | B.14 | C.6 |
| 1.1137 | B.15 | C.6 |
| 1.1138 | B.16 | C.6 |
| 1.1139 | B.17 | C.6 |
| 1.1140 | B.18 | C.6 |
| 1.1141 | B.19 | C.6 |
| 1.1142 | B.20 | C.6 |
| 1.1143 | B.21 | C.6 |
| 1.1144 | B.22 | C.6 |
| 1.1145 | B.23 | C.6 |
| 1.1146 | B.24 | C.6 |
| 1.1147 | B.25 | C.6 |
| 1.1148 | B.26 | C.6 |
| 1.1149 | B.27 | C.6 |
| 1.1150 | B.28 | C.6 |
| 1.1151 | B.29 | C.6 |
| 1.1152 | B.30 | C.6 |
| 1.1153 | B.31 | C.6 |
| 1.1154 | B.32 | C.6 |
| 1.1155 | B.33 | C.6 |
| 1.1156 | B.34 | C.6 |
| 1.1157 | B.35 | C.6 |
| 1.1158 | B.36 | C.6 |
| 1.1159 | B.37 | C.6 |
| 1.1160 | B.38 | C.6 |
| 1.1161 | B.39 | C.6 |
| 1.1162 | B.40 | C.6 |
| 1.1163 | B.41 | C.6 |
| 1.1164 | B.42 | C.6 |
| 1.1165 | B.43 | C.6 |
| 1.1166 | B.44 | C.6 |
| 1.1167 | B.45 | C.6 |
| 1.1168 | B.46 | C.6 |
| 1.1169 | B.47 | C.6 |
| 1.1170 | B.48 | C.6 |
| 1.1171 | B.49 | C.6 |
| 1.1172 | B.50 | C.6 |
| 1.1173 | B.51 | C.6 |
| 1.1174 | B.52 | C.6 |
| 1.1175 | B.53 | C.6 |
| 1.1176 | B.54 | C.6 |
| 1.1177 | B.55 | C.6 |
| 1.1178 | B.56 | C.6 |
| 1.1179 | B.57 | C.6 |
| 1.1180 | B.58. | C.6 |
| 1.1181 | B.59 | C.6 |
| 1.1182 | B.60 | C.6 |
| 1.1183 | B.61 | C.6 |
| 1.1184 | B.62 | C.6 |
| 1.1185 | B.63 | C.6 |
| 1.1186 | B.64 | C.6 |
| 1.1187 | B.65 | C.6 |
| 1.1188 | B.66 | C.6 |
| 1.1189 | B.67 | C.6 |
| 1.1190 | B.68 | C.6 |
| 1.1191 | B.69 | C.6 |
| 1.1192 | B.70 | C.6 |
| 1.1193 | B.71 | C.6 |
| 1.1194 | B.72 | C.6 |
| 1.1195 | B.73 | C.6 |
| 1.1196 | B.74 | C.6 |
| 1.1197 | B.75 | C.6 |
| 1.1198 | B.76 | C.6 |
| 1.1199 | B.77 | C.6 |
| 1.1200 | B.78 | C.6 |
| 1.1201 | B.79 | C.6 |
| 1.1202 | B.80 | C.6 |
| 1.1203 | B.81 | C.6 |
| 1.1204 | B.82 | C.6 |
| 1.1205 | B.83 | C.6 |
| 1.1206 | B.84 | C.6 |
| 1.1207 | B.85 | C.6 |
| 1.1208 | B.86 | C.6 |
| 1.1209 | B.87 | C.6 |
| 1.1210 | B.88 | C.6 |
| 1.1211 | B.89 | C.6 |
| 1.1212 | B.90 | C.6 |
| 1.1213 | B.91 | C.6 |
| 1.1214 | B.92 | C.6 |
| 1.1215 | B.93 | C.6 |
| 1.1216 | B.94 | C.6 |
| 1.1217 | B.95 | C.6 |
| 1.1218 | B.96 | C.6 |
| 1.1219 | B.97 | C.6 |
| 1.1220 | B.98 | C.6 |
| 1.1221 | B.99 | C.6 |
| 1.1222 | B.100 | C.6 |
| 1.1223 | B.101 | C.6 |
| 1.1224 | B.102 | C.6 |
| 1.1225 | B.103 | C.6 |
| 1.1226 | B.104 | C.6 |
| 1.1227 | B.105 | C.6 |
| 1.1228 | B.106 | C.6 |
| 1.1229 | B.107 | C.6 |
| 1.1230 | B.108 | C.6 |
| 1.1231 | B.109 | C.6 |
| 1.1232 | B.110 | C.6 |
| 1.1233 | B.111 | C.6 |
| 1.1234 | B.112 | C.6 |
| 1.1235 | B.113 | C.6 |
| 1.1236 | B.114 | C.6 |
| 1.1237 | B.115 | C.6 |
| 1.1238 | B.116 | C.6 |
| 1.1239 | B.117 | C.6 |
| 1.1240 | B.118 | C.6 |
| 1.1241 | B.119 | C.6 |
| 1.1242 | B.120 | C.6 |
| 1.1243 | B.121 | C.6 |
| 1.1244 | B.122 | C.6 |
| 1.1245 | B.123 | C.6 |
| 1.1246 | B.124 | C.6 |
| 1.1247 | B.125 | C.6 |
| 1.1248 | B.126 | C.6 |
| 1.1249 | B.127 | C.6 |
| 1.1250 | B.128 | C.6 |
| 1.1251 | B.129 | C.6 |
| 1.1252 | B.130 | C.6 |
| 1.1253 | B.131 | C.6 |
| 1.1254 | B.132 | C.6 |
| 1.1255 | B.133 | C.6 |
| 1.1256 | B.134 | C.6 |
| 1.1257 | B.135 | C.6 |
| 1.1258 | B.136 | C.6 |
| 1.1259 | B.137 | C.6 |
| 1.1260 | B.138 | C.6 |
| 1.1261 | B.139 | C.6 |
| 1.1262 | B.140 | C.6 |
| 1.1263 | B.141 | C.6 |
| 1.1264 | B.142 | C.6 |
| 1.1265 | B.143 | C.6 |
| 1.1266 | B.144 | C.6 |
| 1.1267 | B.145 | C.6 |
| 1.1268 | B.146 | C.6 |
| 1.1269 | B.147 | C.6 |
| 1.1270 | B.148 | C.6 |
| 1.1271 | B.149 | C.6 |
| 1.1272 | B.150 | C.6 |
| 1.1273 | B.151 | C.6 |
| 1.1274 | B.152 | C.6 |
| 1.1275 | B.153 | C.6 |
| 1.1276 | B.154 | C.6 |
| 1.1277 | B.155 | C.6 |
| 1.1278 | B.156 | C.6 |
| 1.1279 | B.157 | C.6 |
| 1.1280 | B.158 | C.6 |
| 1.1281 | B.159 | C.6 |
| 1.1282 | B.160 | C.6 |
| 1.1283 | B.161 | C.6 |
| 1.1284 | B.162 | C.6 |
| 1.1285 | B.163 | C.6 |
| 1.1286 | B.164 | C.6 |
| 1.1287 | B.165 | C.6 |
| 1.1288 | B.166 | C.6 |
| 1.1289 | B.167 | C.6 |
| 1.1290 | B.168 | C.6 |
| 1.1291 | B.169 | C.6 |
| 1.1292 | B.170 | C.6 |
| 1.1293 | B.171 | C.6 |
| 1.1294 | B.172 | C.6 |
| 1.1295 | B.173 | C.6 |
| 1.1296 | B.174 | C.6 |
| 1.1297 | B.175 | C.6 |
| 1.1298 | B.176 | C.6 |
| 1.1299 | B.177 | C.6 |
| 1.1300 | B.178 | C.6 |
| 1.1301 | B.179 | C.6 |
| 1.1302 | B.180 | C.6 |
| 1.1303 | B.181 | C.6 |
| 1.1304 | B.182 | C.6 |
| 1.1305 | B.183 | C.6 |
| 1.1306 | B.184 | C.6 |
| 1.1307 | B.185 | C.6 |
| 1.1308 | B.186 | C.6 |
| 1.1309 | B.187 | C.6 |
| 1.1310 | B.1 | C.7 |
| 1.1311 | B.2 | C.7 |
| 1.1312 | B.3 | C.7 |
| 1.1313 | B.4 | C.7 |
| 1.1314 | B.5 | C.7 |
| 1.1315 | B.6 | C.7 |
| 1.1316 | B.7 | C.7 |
| 1.1317 | B.8 | C.7 |
| 1.1318 | B.9 | C.7 |
| 1.1319 | B.10 | C.7 |
| 1.1320 | B.11 | C.7 |
| 1.1321 | B.12 | C.7 |
| 1.1322 | B.13 | C.7 |
| 1.1323 | B.14 | C.7 |
| 1.1324 | B.15 | C.7 |
| 1.1325 | B.16 | C.7 |
| 1.1326 | B.17 | C.7 |
| 1.1327 | B.18 | C.7 |
| 1.1328 | B.19 | C.7 |
| 1.1329 | B.20 | C.7 |
| 1.1330 | B.21 | C.7 |
| 1.1331 | B.22 | C.7 |
| 1.1332 | B.23 | C.7 |
| 1.1333 | B.24 | C.7 |
| 1.1334 | B.25 | C.7 |
| 1.1335 | B.26 | C.7 |
| 1.1336 | B.27 | C.7 |
| 1.1337 | B.28 | C.7 |
| 1.1338 | B.29 | C.7 |
| 1.1339 | B.30 | C.7 |
| 1.1340 | B.31 | C.7 |
| 1.1341 | B.32 | C.7 |
| 1.1342 | B.33 | C.7 |
| 1.1343 | B.34 | C.7 |
| 1.1344 | B.35 | C.7 |
| 1.1345 | B.36 | C.7 |
| 1.1346 | B.37 | C.7 |
| 1.1347 | B.38 | C.7 |
| 1.1348 | B.39 | C.7 |
| 1.1349 | B.40 | C.7 |
| 1.1350 | B.41 | C.7 |
| 1.1351 | B.42 | C.7 |
| 1.1352 | B.43 | C.7 |
| 1.1353 | B.44 | C.7 |
| 1.1354 | B.45 | C.7 |
| 1.1355 | B.46 | C.7 |
| 1.1356 | B.47 | C.7 |
| 1.1357 | B.48 | C.7 |
| 1.1358 | B.49 | C.7 |
| 1.1359 | B.50 | C.7 |
| 1.1360 | B.51 | C.7 |
| 1.1361 | B.52 | C.7 |
| 1.1362 | B.53 | C.7 |
| 1.1363 | B.54 | C.7 |
| 1.1364 | B.55 | C.7 |
| 1.1365 | B.56 | C.7 |
| 1.1366 | B.57 | C.7 |
| 1.1367 | B.58. | C.7 |
| 1.1368 | B.59 | C.7 |
| 1.1369 | B.60 | C.7 |
| 1.1370 | B.61 | C.7 |
| 1.1371 | B.62 | C.7 |
| 1.1372 | B.63 | C.7 |
| 1.1373 | B.64 | C.7 |
| 1.1374 | B.65 | C.7 |
| 1.1375 | B.66 | C.7 |
| 1.1376 | B.67 | C.7 |
| 1.1377 | B.68 | C.7 |
| 1.1378 | B.69 | C.7 |
| 1.1379 | B.70 | C.7 |
| 1.1380 | B.71 | C.7 |
| 1.1381 | B.72 | C.7 |
| 1.1382 | B.73 | C.7 |
| 1.1383 | B.74 | C.7 |
| 1.1384 | B.75 | C.7 |
| 1.1385 | B.76 | C.7 |
| 1.1386 | B.77 | C.7 |
| 1.1387 | B.78 | C.7 |
| 1.1388 | B.79 | C.7 |
| 1.1389 | B.80 | C.7 |
| 1.1390 | B.81 | C.7 |
| 1.1391 | B.82 | C.7 |
| 1.1392 | B.83 | C.7 |
| 1.1393 | B.84 | C.7 |
| 1.1394 | B.85 | C.7 |
| 1.1395 | B.86 | C.7 |
| 1.1396 | B.87 | C.7 |
| 1.1397 | B.88 | C.7 |
| 1.1398 | B.89 | C.7 |
| 1.1399 | B.90 | C.7 |
| 1.1400 | B.91 | C.7 |
| 1.1401 | B.92 | C.7 |
| 1.1402 | B.93 | C.7 |
| 1.1403 | B.94 | C.7 |
| 1.1404 | B.95 | C.7 |
| 1.1405 | B.96 | C.7 |
| 1.1406 | B.97 | C.7 |
| 1.1407 | B.98 | C.7 |
| 1.1408 | B.99 | C.7 |
| 1.1409 | B.100 | C.7 |
| 1.1410 | B.101 | C.7 |
| 1.1411 | B.102 | C.7 |
| 1.1412 | B.103 | C.7 |
| 1.1413 | B.104 | C.7 |
| 1.1414 | B.105 | C.7 |
| 1.1415 | B.106 | C.7 |
| 1.1416 | B.107 | C.7 |
| 1.1417 | B.108 | C.7 |
| 1.1418 | B.109 | C.7 |
| 1.1419 | B.110 | C.7 |
| 1.1420 | B.111 | C.7 |
| 1.1421 | B.112 | C.7 |
| 1.1422 | B.113 | C.7 |
| 1.1423 | B.114 | C.7 |
| 1.1424 | B.115 | C.7 |
| 1.1425 | B.116 | C.7 |
| 1.1426 | B.117 | C.7 |
| 1.1427 | B.118 | C.7 |
| 1.1428 | B.119 | C.7 |
| 1.1429 | B.120 | C.7 |
| 1.1430 | B.121 | C.7 |
| 1.1431 | B.122 | C.7 |
| 1.1432 | B.123 | C.7 |
| 1.1433 | B.124 | C.7 |
| 1.1434 | B.125 | C.7 |
| 1.1435 | B.126 | C.7 |
| 1.1436 | B.127 | C.7 |
| 1.1437 | B.128 | C.7 |
| 1.1438 | B.129 | C.7 |
| 1.1439 | B.130 | C.7 |
| 1.1440 | B.131 | C.7 |
| 1.1441 | B.132 | C.7 |
| 1.1442 | B.133 | C.7 |
| 1.1443 | B.134 | C.7 |
| 1.1444 | B.135 | C.7 |
| 1.1445 | B.136 | C.7 |
| 1.1446 | B.137 | C.7 |
| 1.1447 | B.138 | C.7 |
| 1.1448 | B.139 | C.7 |
| 1.1449 | B.140 | C.7 |
| 1.1450 | B.141 | C.7 |
| 1.1451 | B.142 | C.7 |
| 1.1452 | B.143 | C.7 |
| 1.1453 | B.144 | C.7 |
| 1.1454 | B.145 | C.7 |
| 1.1455 | B.146 | C.7 |
| 1.1456 | B.147 | C.7 |
| 1.1457 | B.148 | C.7 |
| 1.1458 | B.149 | C.7 |
| 1.1459 | B.150 | C.7 |
| 1.1460 | B.151 | C.7 |
| 1.1461 | B.152 | C.7 |
| 1.1462 | B.153 | C.7 |
| 1.1463 | B.154 | C.7 |
| 1.1464 | B.155 | C.7 |
| 1.1465 | B.156 | C.7 |
| 1.1466 | B.157 | C.7 |
| 1.1467 | B.158 | C.7 |
| 1.1468 | B.159 | C.7 |
| 1.1469 | B.160 | C.7 |
| 1.1470 | B.161 | C.7 |
| 1.1471 | B.162 | C.7 |
| 1.1472 | B.163 | C.7 |
| 1.1473 | B.164 | C.7 |
| 1.1474 | B.165 | C.7 |
| 1.1475 | B.166 | C.7 |
| 1.1476 | B.167 | C.7 |
| 1.1477 | B.168 | C.7 |
| 1.1478 | B.169 | C.7 |
| 1.1479 | B.170 | C.7 |
| 1.1480 | B.171 | C.7 |
| 1.1481 | B.172 | C.7 |
| 1.1482 | B.173 | C.7 |
| 1.1483 | B.174 | C.7 |
| 1.1484 | B.175 | C.7 |
| 1.1485 | B.176 | C.7 |
| 1.1486 | B.177 | C.7 |
| 1.1487 | B.178 | C.7 |
| 1.1488 | B.179 | C.7 |
| 1.1489 | B.180 | C.7 |
| 1.1490 | B.181 | C.7 |
| 1.1491 | B.182 | C.7 |
| 1.1492 | B.183 | C.7 |
| 1.1493 | B.184 | C.7 |
| 1.1494 | B.185 | C.7 |
| 1.1495 | B.186 | C.7 |
| 1.1496 | B.187 | C.7 |
| 1.1497 | B.1 | C.8 |
| 1.1498 | B.2 | C.8 |
| 1.1499 | B.3 | C.8 |
| 1.1500 | B.4 | C.8 |
| 1.1501 | B.5 | C.8 |
| 1.1502 | B.6 | C.8 |
| 1.1503 | B.7 | C.8 |
| 1.1504 | B.8 | C.8 |
| 1.1505 | B.9 | C.8 |
| 1.1506 | B.10 | C.8 |
| 1.1507 | B.11 | C.8 |
| 1.1508 | B.12 | C.8 |
| 1.1509 | B.13 | C.8 |
| 1.1510 | B.14 | C.8 |
| 1.1511 | B.15 | C.8 |
| 1.1512 | B.16 | C.8 |
| 1.1513 | B.17 | C.8 |
| 1.1514 | B.18 | C.8 |
| 1.1515 | B.19 | C.8 |
| 1.1516 | B.20 | C.8 |
| 1.1517 | B.21 | C.8 |
| 1.1518 | B.22 | C.8 |
| 1.1519 | B.23 | C.8 |
| 1.1520 | B.24 | C.8 |
| 1.1521 | B.25 | C.8 |
| 1.1522 | B.26 | C.8 |
| 1.1523 | B.27 | C.8 |
| 1.1524 | B.28 | C.8 |
| 1.1525 | B.29 | C.8 |
| 1.1526 | B.30 | C.8 |
| 1.1527 | B.31 | C.8 |
| 1.1528 | B.32 | C.8 |
| 1.1529 | B.33 | C.8 |
| 1.1530 | B.34 | C.8 |
| 1.1531 | B.35 | C.8 |
| 1.1532 | B.36 | C.8 |
| 1.1533 | B.37 | C.8 |
| 1.1534 | B.38 | C.8 |
| 1.1535 | B.39 | C.8 |
| 1.1536 | B.40 | C.8 |
| 1.1537 | B.41 | C.8 |
| 1.1538 | B.42 | C.8 |
| 1.1539 | B.43 | C.8 |
| 1.1540 | B.44 | C.8 |
| 1.1541 | B.45 | C.8 |
| 1.1542 | B.46 | C.8 |
| 1.1543 | B.47 | C.8 |
| 1.1544 | B.48 | C.8 |
| 1.1545 | B.49 | C.8 |
| 1.1546 | B.50 | C.8 |
| 1.1547 | B.51 | C.8 |
| 1.1548 | B.52 | C.8 |
| 1.1549 | B.53 | C.8 |
| 1.1550 | B.54 | C.8 |
| 1.1551 | B.55 | C.8 |
| 1.1552 | B.56 | C.8 |
| 1.1553 | B.57 | C.8 |
| 1.1554 | B.58. | C.8 |
| 1.1555 | B.59 | C.8 |
| 1.1556 | B.60 | C.8 |
| 1.1557 | B.61 | C.8 |
| 1.1558 | B.62 | C.8 |
| 1.1559 | B.63 | C.8 |
| 1.1560 | B.64 | C.8 |
| 1.1561 | B.65 | C.8 |
| 1.1562 | B.66 | C.8 |
| 1.1563 | B.67 | C.8 |
| 1.1564 | B.68 | C.8 |
| 1.1565 | B.69 | C.8 |
| 1.1566 | B.70 | C.8 |
| 1.1567 | B.71 | C.8 |
| 1.1568 | B.72 | C.8 |
| 1.1569 | B.73 | C.8 |
| 1.1570 | B.74 | C.8 |
| 1.1571 | B.75 | C.8 |
| 1.1572 | B.76 | C.8 |
| 1.1573 | B.77 | C.8 |
| 1.1574 | B.78 | C.8 |
| 1.1575 | B.79 | C.8 |
| 1.1576 | B.80 | C.8 |
| 1.1577 | B.81 | C.8 |
| 1.1578 | B.82 | C.8 |
| 1.1579 | B.83 | C.8 |
| 1.1580 | B.84 | C.8 |
| 1.1581 | B.85 | C.8 |
| 1.1582 | B.86 | C.8 |
| 1.1583 | B.87 | C.8 |
| 1.1584 | B.88 | C.8 |
| 1.1585 | B.89 | C.8 |
| 1.1586 | B.90 | C.8 |
| 1.1587 | B.91 | C.8 |
| 1.1588 | B.92 | C.8 |
| 1.1589 | B.93 | C.8 |
| 1.1590 | B.94 | C.8 |
| 1.1591 | B.95 | C.8 |
| 1.1592 | B.96 | C.8 |
| 1.1593 | B.97 | C.8 |
| 1.1594 | B.98 | C.8 |
| 1.1595 | B.99 | C.8 |
| 1.1596 | B.100 | C.8 |
| 1.1597 | B.101 | C.8 |
| 1.1598 | B.102 | C.8 |
| 1.1599 | B.103 | C.8 |
| 1.1600 | B.104 | C.8 |
| 1.1601 | B.105 | C.8 |
| 1.1602 | B.106 | C.8 |
| 1.1603 | B.107 | C.8 |
| 1.1604 | B.108 | C.8 |
| 1.1605 | B.109 | C.8 |
| 1.1606 | B.110 | C.8 |
| 1.1607 | B.111 | C.8 |
| 1.1608 | B.112 | C.8 |
| 1.1609 | B.113 | C.8 |
| 1.1610 | B.114 | C.8 |
| 1.1611 | B.115 | C.8 |
| 1.1612 | B.116 | C.8 |
| 1.1613 | B.117 | C.8 |
| 1.1614 | B.118 | C.8 |
| 1.1615 | B.119 | C.8 |
| 1.1616 | B.120 | C.8 |
| 1.1617 | B.121 | C.8 |
| 1.1618 | B.122 | C.8 |
| 1.1619 | B.123 | C.8 |
| 1.1620 | B.124 | C.8 |
| 1.1621 | B.125 | C.8 |
| 1.1622 | B.126 | C.8 |
| 1.1623 | B.127 | C.8 |
| 1.1624 | B.128 | C.8 |
| 1.1625 | B.129 | C.8 |
| 1.1626 | B.130 | C.8 |
| 1.1627 | B.131 | C.8 |
| 1.1628 | B.132 | C.8 |
| 1.1629 | B.133 | C.8 |
| 1.1630 | B.134 | C.8 |
| 1.1631 | B.135 | C.8 |
| 1.1632 | B.136 | C.8 |
| 1.1633 | B.137 | C.8 |
| 1.1634 | B.138 | C.8 |
| 1.1635 | B.139 | C.8 |
| 1.1636 | B.140 | C.8 |
| 1.1637 | B.141 | C.8 |
| 1.1638 | B.142 | C.8 |
| 1.1639 | B.143 | C.8 |
| 1.1640 | B.144 | C.8 |
| 1.1641 | B.145 | C.8 |
| 1.1642 | B.146 | C.8 |
| 1.1643 | B.147 | C.8 |
| 1.1644 | B.148 | C.8 |
| 1.1645 | B.149 | C.8 |
| 1.1646 | B.150 | C.8 |
| 1.1647 | B.151 | C.8 |
| 1.1648 | B.152 | C.8 |
| 1.1649 | B.153 | C.8 |
| 1.1650 | B.154 | C.8 |
| 1.1651 | B.155 | C.8 |
| 1.1652 | B.156 | C.8 |
| 1.1653 | B.157 | C.8 |
| 1.1654 | B.158 | C.8 |
| 1.1655 | B.159 | C.8 |
| 1.1656 | B.160 | C.8 |
| 1.1657 | B.161 | C.8 |
| 1.1658 | B.162 | C.8 |
| 1.1659 | B.163 | C.8 |
| 1.1660 | B.164 | C.8 |
| 1.1661 | B.165 | C.8 |
| 1.1662 | B.166 | C.8 |
| 1.1663 | B.167 | C.8 |
| 1.1664 | B.168 | C.8 |
| 1.1665 | B.169 | C.8 |
| 1.1666 | B.170 | C.8 |
| 1.1667 | B.171 | C.8 |
| 1.1668 | B.172 | C.8 |
| 1.1669 | B.173 | C.8 |
| 1.1670 | B.174 | C.8 |
| 1.1671 | B.175 | C.8 |
| 1.1672 | B.176 | C.8 |
| 1.1673 | B.177 | C.8 |
| 1.1674 | B.178 | C.8 |
| 1.1675 | B.179 | C.8 |
| 1.1676 | B.180 | C.8 |
| 1.1677 | B.181 | C.8 |
| 1.1678 | B.182 | C.8 |
| 1.1679 | B.183 | C.8 |
| 1.1680 | B.184 | C.8 |
| 1.1681 | B.185 | C.8 |
| 1.1682 | B.186 | C.8 |
| 1.1683 | B.187 | C.8 |
| 1.1684 | B.1 | C.9 |
| 1.1685 | B.2 | C.9 |
| 1.1686 | B.3 | C.9 |
| 1.1687 | B.4 | C.9 |
| 1.1688 | B.5 | C.9 |
| 1.1689 | B.6 | C.9 |
| 1.1690 | B.7 | C.9 |
| 1.1691 | B.8 | C.9 |
| 1.1692 | B.9 | C.9 |
| 1.1693 | B.10 | C.9 |
| 1.1694 | B.11 | C.9 |
| 1.1695 | B.12 | C.9 |
| 1.1696 | B.13 | C.9 |
| 1.1697 | B.14 | C.9 |
| 1.1698 | B.15 | C.9 |
| 1.1699 | B.16 | C.9 |
| 1.1700 | B.17 | C.9 |
| 1.1701 | B.18 | C.9 |
| 1.1702 | B.19 | C.9 |
| 1.1703 | B.20 | C.9 |
| 1.1704 | B.21 | C.9 |
| 1.1705 | B.22 | C.9 |
| 1.1706 | B.23 | C.9 |
| 1.1707 | B.24 | C.9 |
| 1.1708 | B.25 | C.9 |
| 1.1709 | B.26 | C.9 |
| 1.1710 | B.27 | C.9 |
| 1.1711 | B.28 | C.9 |
| 1.1712 | B.29 | C.9 |
| 1.1713 | B.30 | C.9 |
| 1.1714 | B.31 | C.9 |
| 1.1715 | B.32 | C.9 |
| 1.1716 | B.33 | C.9 |
| 1.1717 | B.34 | C.9 |
| 1.1718 | B.35 | C.9 |
| 1.1719 | B.36 | C.9 |
| 1.1720 | B.37 | C.9 |
| 1.1721 | B.38 | C.9 |
| 1.1722 | B.39 | C.9 |
| 1.1723 | B.40 | C.9 |
| 1.1724 | B.41 | C.9 |
| 1.1725 | B.42 | C.9 |
| 1.1726 | B.43 | C.9 |
| 1.1727 | B.44 | C.9 |
| 1.1728 | B.45 | C.9 |
| 1.1729 | B.46 | C.9 |
| 1.1730 | B.47 | C.9 |
| 1.1731 | B.48 | C.9 |
| 1.1732 | B.49 | C.9 |
| 1.1733 | B.50 | C.9 |
| 1.1734 | B.51 | C.9 |
| 1.1735 | B.52 | C.9 |
| 1.1736 | B.53 | C.9 |
| 1.1737 | B.54 | C.9 |
| 1.1738 | B.55 | C.9 |
| 1.1739 | B.56 | C.9 |
| 1.1740 | B.57 | C.9 |
| 1.1741 | B.58. | C.9 |
| 1.1742 | B.59 | C.9 |
| 1.1743 | B.60 | C.9 |
| 1.1744 | B.61 | C.9 |
| 1.1745 | B.62 | C.9 |
| 1.1746 | B.63 | C.9 |
| 1.1747 | B.64 | C.9 |
| 1.1748 | B.65 | C.9 |
| 1.1749 | B.66 | C.9 |
| 1.1750 | B.67 | C.9 |
| 1.1751 | B.68 | C.9 |
| 1.1752 | B.69 | C.9 |
| 1.1753 | B.70 | C.9 |
| 1.1754 | B.71 | C.9 |
| 1.1755 | B.72 | C.9 |
| 1.1756 | B.73 | C.9 |
| 1.1757 | B.74 | C.9 |
| 1.1758 | B.75 | C.9 |
| 1.1759 | B.76 | C.9 |
| 1.1760 | B.77 | C.9 |
| 1.1761 | B.78 | C.9 |
| 1.1762 | B.79 | C.9 |
| 1.1763 | B.80 | C.9 |
| 1.1764 | B.81 | C.9 |
| 1.1765 | B.82 | C.9 |
| 1.1766 | B.83 | C.9 |
| 1.1767 | B.84 | C.9 |
| 1.1768 | B.85 | C.9 |
| 1.1769 | B.86 | C.9 |
| 1.1770 | B.87 | C.9 |
| 1.1771 | B.88 | C.9 |
| 1.1772 | B.89 | C.9 |
| 1.1773 | B.90 | C.9 |
| 1.1774 | B.91 | C.9 |
| 1.1775 | B.92 | C.9 |
| 1.1776 | B.93 | C.9 |
| 1.1777 | B.94 | C.9 |
| 1.1778 | B.95 | C.9 |
| 1.1779 | B.96 | C.9 |
| 1.1780 | B.97 | C.9 |
| 1.1781 | B.98 | C.9 |
| 1.1782 | B.99 | C.9 |
| 1.1783 | B.100 | C.9 |
| 1.1784 | B.101 | C.9 |
| 1.1785 | B.102 | C.9 |
| 1.1786 | B.103 | C.9 |
| 1.1787 | B.104 | C.9 |
| 1.1788 | B.105 | C.9 |
| 1.1789 | B.106 | C.9 |
| 1.1790 | B.107 | C.9 |
| 1.1791 | B.108 | C.9 |
| 1.1792 | B.109 | C.9 |
| 1.1793 | B.110 | C.9 |
| 1.1794 | B.111 | C.9 |
| 1.1795 | B.112 | C.9 |
| 1.1796 | B.113 | C.9 |
| 1.1797 | B.114 | C.9 |
| 1.1798 | B.115 | C.9 |
| 1.1799 | B.116 | C.9 |
| 1.1800 | B.117 | C.9 |
| 1.1801 | B.118 | C.9 |
| 1.1802 | B.119 | C.9 |
| 1.1803 | B.120 | C.9 |
| 1.1804 | B.121 | C.9 |
| 1.1805 | B.122 | C.9 |
| 1.1806 | B.123 | C.9 |
| 1.1807 | B.124 | C.9 |
| 1.1808 | B.125 | C.9 |
| 1.1809 | B.126 | C.9 |
| 1.1810 | B.127 | C.9 |
| 1.1811 | B.128 | C.9 |
| 1.1812 | B.129 | C.9 |
| 1.1813 | B.130 | C.9 |
| 1.1814 | B.131 | C.9 |
| 1.1815 | B.132 | C.9 |
| 1.1816 | B.133 | C.9 |
| 1.1817 | B.134 | C.9 |
| 1.1818 | B.135 | C.9 |
| 1.1819 | B.136 | C.9 |
| 1.1820 | B.137 | C.9 |
| 1.1821 | B.138 | C.9 |
| 1.1822 | B.139 | C.9 |
| 1.1823 | B.140 | C.9 |
| 1.1824 | B.141 | C.9 |
| 1.1825 | B.142 | C.9 |
| 1.1826 | B.143 | C.9 |
| 1.1827 | B.144 | C.9 |
| 1.1828 | B.145 | C.9 |
| 1.1829 | B.146 | C.9 |
| 1.1830 | B.147 | C.9 |
| 1.1831 | B.148 | C.9 |
| 1.1832 | B.149 | C.9 |
| 1.1833 | B.150 | C.9 |
| 1.1834 | B.151 | C.9 |
| 1.1835 | B.152 | C.9 |
| 1.1836 | B.153 | C.9 |
| 1.1837 | B.154 | C.9 |
| 1.1838 | B.155 | C.9 |
| 1.1839 | B.156 | C.9 |
| 1.1840 | B.157 | C.9 |
| 1.1841 | B.158 | C.9 |
| 1.1842 | B.159 | C.9 |
| 1.1843 | B.160 | C.9 |
| 1.1844 | B.161 | C.9 |
| 1.1845 | B.162 | C.9 |
| 1.1846 | B.163 | C.9 |
| 1.1847 | B.164 | C.9 |
| 1.1848 | B.165 | C.9 |
| 1.1849 | B.166 | C.9 |
| 1.1850 | B.167 | C.9 |
| 1.1851 | B.168 | C.9 |
| 1.1852 | B.169 | C.9 |
| 1.1853 | B.170 | C.9 |
| 1.1854 | B.171 | C.9 |
| 1.1855 | B.172 | C.9 |
| 1.1856 | B.173 | C.9 |
| 1.1857 | B.174 | C.9 |
| 1.1858 | B.175 | C.9 |
| 1.1859 | B.176 | C.9 |
| 1.1860 | B.177 | C.9 |
| 1.1861 | B.178 | C.9 |
| 1.1862 | B.179 | C.9 |
| 1.1863 | B.180 | C.9 |
| 1.1864 | B.181 | C.9 |
| 1.1865 | B.182 | C.9 |
| 1.1866 | B.183 | C.9 |
| 1.1867 | B.184 | C.9 |
| 1.1868 | B.185 | C.9 |
| 1.1869 | B.186 | C.9 |
| 1.1870 | B.187 | C.9 |
| 1.1871 | B.1 | C.10 |
| 1.1872 | B.2 | C.10 |
| 1.1873 | B.3 | C.10 |
| 1.1874 | B.4 | C.10 |
| 1.1875 | B.5 | C.10 |
| 1.1876 | B.6 | C.10 |
| 1.1877 | B.7 | C.10 |
| 1.1878 | B.8 | C.10 |
| 1.1879 | B.9 | C.10 |
| 1.1880 | B.10 | C.10 |
| 1.1881 | B.11 | C.10 |
| 1.1882 | B.12 | C.10 |
| 1.1883 | B.13 | C.10 |
| 1.1884 | B.14 | C.10 |
| 1.1885 | B.15 | C.10 |
| 1.1886 | B.16 | C.10 |
| 1.1887 | B.17 | C.10 |
| 1.1888 | B.18 | C.10 |
| 1.1889 | B.19 | C.10 |
| 1.1890 | B.20 | C.10 |
| 1.1891 | B.21 | C.10 |
| 1.1892 | B.22 | C.10 |
| 1.1893 | B.23 | C.10 |
| 1.1894 | B.24 | C.10 |
| 1.1895 | B.25 | C.10 |
| 1.1896 | B.26 | C.10 |
| 1.1897 | B.27 | C.10 |
| 1.1898 | B.28 | C.10 |
| 1.1899 | B.29 | C.10 |
| 1.1900 | B.30 | C.10 |
| 1.1901 | B.31 | C.10 |
| 1.1902 | B.32 | C.10 |
| 1.1903 | B.33 | C.10 |
| 1.1904 | B.34 | C.10 |
| 1.1905 | B.35 | C.10 |
| 1.1906 | B.36 | C.10 |
| 1.1907 | B.37 | C.10 |
| 1.1908 | B.38 | C.10 |
| 1.1909 | B.39 | C.10 |
| 1.1910 | B.40 | C.10 |
| 1.1911 | B.41 | C.10 |
| 1.1912 | B.42 | C.10 |
| 1.1913 | B.43 | C.10 |
| 1.1914 | B.44 | C.10 |
| 1.1915 | B.45 | C.10 |
| 1.1916 | B.46 | C.10 |
| 1.1917 | B.47 | C.10 |
| 1.1918 | B.48 | C.10 |
| 1.1919 | B.49 | C.10 |
| 1.1920 | B.50 | C.10 |
| 1.1921 | B.51 | C.10 |
| 1.1922 | B.52 | C.10 |
| 1.1923 | B.53 | C.10 |
| 1.1924 | B.54 | C.10 |
| 1.1925 | B.55 | C.10 |
| 1.1926 | B.56 | C.10 |
| 1.1927 | B.57 | C.10 |
| 1.1928 | B.58. | C.10 |
| 1.1929 | B.59 | C.10 |
| 1.1930 | B.60 | C.10 |
| 1.1931 | B.61 | C.10 |
| 1.1932 | B.62 | C.10 |
| 1.1933 | B.63 | C.10 |
| 1.1934 | B.64 | C.10 |
| 1.1935 | B.65 | C.10 |
| 1.1936 | B.66 | C.10 |
| 1.1937 | B.67 | C.10 |
| 1.1938 | B.68 | C.10 |
| 1.1939 | B.69 | C.10 |
| 1.1940 | B.70 | C.10 |
| 1.1941 | B.71 | C.10 |
| 1.1942 | B.72 | C.10 |
| 1.1943 | B.73 | C.10 |
| 1.1944 | B.74 | C.10 |
| 1.1945 | B.75 | C.10 |
| 1.1946 | B.76 | C.10 |
| 1.1947 | B.77 | C.10 |
| 1.1948 | B.78 | C.10 |
| 1.1949 | B.79 | C.10 |
| 1.1950 | B.80 | C.10 |
| 1.1951 | B.81 | C.10 |
| 1.1952 | B.82 | C.10 |
| 1.1953 | B.83 | C.10 |
| 1.1954 | B.84 | C.10 |
| 1.1955 | B.85 | C.10 |
| 1.1956 | B.86 | C.10 |
| 1.1957 | B.87 | C.10 |
| 1.1958 | B.88 | C.10 |
| 1.1959 | B.89 | C.10 |
| 1.1960 | B.90 | C.10 |
| 1.1961 | B.91 | C.10 |
| 1.1962 | B.92 | C.10 |
| 1.1963 | B.93 | C.10 |
| 1.1964 | B.94 | C.10 |
| 1.1965 | B.95 | C.10 |
| 1.1966 | B.96 | C.10 |
| 1.1967 | B.97 | C.10 |
| 1.1968 | B.98 | C.10 |
| 1.1969 | B.99 | C.10 |
| 1.1970 | B.100 | C.10 |
| 1.1971 | B.101 | C.10 |
| 1.1972 | B.102 | C.10 |
| 1.1973 | B.103 | C.10 |
| 1.1974 | B.104 | C.10 |
| 1.1975 | B.105 | C.10 |
| 1.1976 | B.106 | C.10 |
| 1.1977 | B.107 | C.10 |
| 1.1978 | B.108 | C.10 |
| 1.1979 | B.109 | C.10 |
| 1.1980 | B.110 | C.10 |
| 1.1981 | B.111 | C.10 |
| 1.1982 | B.112 | C.10 |
| 1.1983 | B.113 | C.10 |
| 1.1984 | B.114 | C.10 |
| 1.1985 | B.115 | C.10 |
| 1.1986 | B.116 | C.10 |
| 1.1987 | B.117 | C.10 |
| 1.1988 | B.118 | C.10 |
| 1.1989 | B.119 | C.10 |
| 1.1990 | B.120 | C.10 |
| 1.1991 | B.121 | C.10 |
| 1.1992 | B.122 | C.10 |
| 1.1993 | B.123 | C.10 |
| 1.1994 | B.124 | C.10 |
| 1.1995 | B.125 | C.10 |
| 1.1996 | B.126 | C.10 |
| 1.1997 | B.127 | C.10 |
| 1.1998 | B.128 | C.10 |
| 1.1999 | B.129 | C.10 |
| 1.2000 | B.130 | C.10 |
| 1.2001 | B.131 | C.10 |
| 1.2002 | B.132 | C.10 |
| 1.2003 | B.133 | C.10 |
| 1.2004 | B.134 | C.10 |
| 1.2005 | B.135 | C.10 |
| 1.2006 | B.136 | C.10 |
| 1.2007 | B.137 | C.10 |
| 1.2008 | B.138 | C.10 |
| 1.2009 | B.139 | C.10 |
| 1.2010 | B.140 | C.10 |
| 1.2011 | B.141 | C.10 |
| 1.2012 | B.142 | C.10 |
| 1.2013 | B.143 | C.10 |
| 1.2014 | B.144 | C.10 |
| 1.2015 | B.145 | C.10 |
| 1.2016 | B.146 | C.10 |
| 1.2017 | B.147 | C.10 |
| 1.2018 | B.148 | C.10 |
| 1.2019 | B.149 | C.10 |
| 1.2020 | B.150 | C.10 |
| 1.2021 | B.151 | C.10 |
| 1.2022 | B.152 | C.10 |
| 1.2023 | B.153 | C.10 |
| 1.2024 | B.154 | C.10 |
| 1.2025 | B.155 | C.10 |
| 1.2026 | B.156 | C.10 |
| 1.2027 | B.157 | C.10 |
| 1.2028 | B.158 | C.10 |
| 1.2029 | B.159 | C.10 |
| 1.2030 | B.160 | C.10 |
| 1.2031 | B.161 | C.10 |
| 1.2032 | B.162 | C.10 |
| 1.2033 | B.163 | C.10 |
| 1.2034 | B.164 | C.10 |
| 1.2035 | B.165 | C.10 |
| 1.2036 | B.166 | C.10 |
| 1.2037 | B.167 | C.10 |
| 1.2038 | B.168 | C.10 |
| 1.2039 | B.169 | C.10 |
| 1.2040 | B.170 | C.10 |
| 1.2041 | B.171 | C.10 |
| 1.2042 | B.172 | C.10 |
| 1.2043 | B.173 | C.10 |
| 1.2044 | B.174 | C.10 |
| 1.2045 | B.175 | C.10 |
| 1.2046 | B.176 | C.10 |
| 1.2047 | B.177 | C.10 |
| 1.2048 | B.178 | C.10 |
| 1.2049 | B.179 | C.10 |
| 1.2050 | B.180 | C.10 |
| 1.2051 | B.181 | C.10 |
| 1.2052 | B.182 | C.10 |
| 1.2053 | B.183 | C.10 |
| 1.2054 | B.184 | C.10 |
| 1.2055 | B.185 | C.10 |
| 1.2056 | B.186 | C.10 |
| 1.2057 | B.187 | C.10 |
| 1.2058 | B.1 | C.11 |
| 1.2059 | B.2 | C.11 |
| 1.2060 | B.3 | C.11 |
| 1.2061 | B.4 | C.11 |
| 1.2062 | B.5 | C.11 |
| 1.2063 | B.6 | C.11 |
| 1.2064 | B.7 | C.11 |
| 1.2065 | B.8 | C.11 |
| 1.2066 | B.9 | C.11 |
| 1.2067 | B.10 | C.11 |
| 1.2068 | B.11 | C.11 |
| 1.2069 | B.12 | C.11 |
| 1.2070 | B.13 | C.11 |
| 1.2071 | B.14 | C.11 |
| 1.2072 | B.15 | C.11 |
| 1.2073 | B.16 | C.11 |
| 1.2074 | B.17 | C.11 |
| 1.2075 | B.18 | C.11 |
| 1.2076 | B.19 | C.11 |
| 1.2077 | B.20 | C.11 |
| 1.2078 | B.21 | C.11 |
| 1.2079 | B.22 | C.11 |
| 1.2080 | B.23 | C.11 |
| 1.2081 | B.24 | C.11 |
| 1.2082 | B.25 | C.11 |
| 1.2083 | B.26 | C.11 |
| 1.2084 | B.27 | C.11 |
| 1.2085 | B.28 | C.11 |
| 1.2086 | B.29 | C.11 |
| 1.2087 | B.30 | C.11 |
| 1.2088 | B.31 | C.11 |
| 1.2089 | B.32 | C.11 |
| 1.2090 | B.33 | C.11 |
| 1.2091 | B.34 | C.11 |
| 1.2092 | B.35 | C.11 |
| 1.2093 | B.36 | C.11 |
| 1.2094 | B.37 | C.11 |
| 1.2095 | B.38 | C.11 |
| 1.2096 | B.39 | C.11 |
| 1.2097 | B.40 | C.11 |
| 1.2098 | B.41 | C.11 |
| 1.2099 | B.42 | C.11 |
| 1.2100 | B.43 | C.11 |
| 1.2101 | B.44 | C.11 |
| 1.2102 | B.45 | C.11 |
| 1.2103 | B.46 | C.11 |
| 1.2104 | B.47 | C.11 |
| 1.2105 | B.48 | C.11 |
| 1.2106 | B.49 | C.11 |
| 1.2107 | B.50 | C.11 |
| 1.2108 | B.51 | C.11 |
| 1.2109 | B.52 | C.11 |
| 1.2110 | B.53 | C.11 |
| 1.2111 | B.54 | C.11 |
| 1.2112 | B.55 | C.11 |
| 1.2113 | B.56 | C.11 |
| 1.2114 | B.57 | C.11 |
| 1.2115 | B.58. | C.11 |
| 1.2116 | B.59 | C.11 |
| 1.2117 | B.60 | C.11 |
| 1.2118 | B.61 | C.11 |
| 1.2119 | B.62 | C.11 |
| 1.2120 | B.63 | C.11 |
| 1.2121 | B.64 | C.11 |
| 1.2122 | B.65 | C.11 |
| 1.2123 | B.66 | C.11 |
| 1.2124 | B.67 | C.11 |
| 1.2125 | B.68 | C.11 |
| 1.2126 | B.69 | C.11 |
| 1.2127 | B.70 | C.11 |
| 1.2128 | B.71 | C.11 |
| 1.2129 | B.72 | C.11 |
| 1.2130 | B.73 | C.11 |
| 1.2131 | B.74 | C.11 |
| 1.2132 | B.75 | C.11 |
| 1.2133 | B.76 | C.11 |
| 1.2134 | B.77 | C.11 |
| 1.2135 | B.78 | C.11 |
| 1.2136 | B.79 | C.11 |
| 1.2137 | B.80 | C.11 |
| 1.2138 | B.81 | C.11 |
| 1.2139 | B.82 | C.11 |
| 1.2140 | B.83 | C.11 |
| 1.2141 | B.84 | C.11 |
| 1.2142 | B.85 | C.11 |
| 1.2143 | B.86 | C.11 |
| 1.2144 | B.87 | C.11 |
| 1.2145 | B.88 | C.11 |
| 1.2146 | B.89 | C.11 |
| 1.2147 | B.90 | C.11 |
| 1.2148 | B.91 | C.11 |
| 1.2149 | B.92 | C.11 |
| 1.2150 | B.93 | C.11 |
| 1.2151 | B.94 | C.11 |
| 1.2152 | B.95 | C.11 |
| 1.2153 | B.96 | C.11 |
| 1.2154 | B.97 | C.11 |
| 1.2155 | B.98 | C.11 |
| 1.2156 | B.99 | C.11 |
| 1.2157 | B.100 | C.11 |
| 1.2158 | B.101 | C.11 |
| 1.2159 | B.102 | C.11 |
| 1.2160 | B.103 | C.11 |
| 1.2161 | B.104 | C.11 |
| 1.2162 | B.105 | C.11 |
| 1.2163 | B.106 | C.11 |
| 1.2164 | B.107 | C.11 |
| 1.2165 | B.108 | C.11 |
| 1.2166 | B.109 | C.11 |
| 1.2167 | B.110 | C.11 |
| 1.2168 | B.111 | C.11 |
| 1.2169 | B.112 | C.11 |
| 1.2170 | B.113 | C.11 |
| 1.2171 | B.114 | C.11 |
| 1.2172 | B.115 | C.11 |
| 1.2173 | B.116 | C.11 |
| 1.2174 | B.117 | C.11 |
| 1.2175 | B.118 | C.11 |
| 1.2176 | B.119 | C.11 |
| 1.2177 | B.120 | C.11 |
| 1.2178 | B.121 | C.11 |
| 1.2179 | B.122 | C.11 |
| 1.2180 | B.123 | C.11 |
| 1.2181 | B.124 | C.11 |
| 1.2182 | B.125 | C.11 |
| 1.2183 | B.126 | C.11 |
| 1.2184 | B.127 | C.11 |
| 1.2185 | B.128 | C.11 |
| 1.2186 | B.129 | C.11 |
| 1.2187 | B.130 | C.11 |
| 1.2188 | B.131 | C.11 |
| 1.2189 | B.132 | C.11 |
| 1.2190 | B.133 | C.11 |
| 1.2191 | B.134 | C.11 |
| 1.2192 | B.135 | C.11 |
| 1.2193 | B.136 | C.11 |
| 1.2194 | B.137 | C.11 |
| 1.2195 | B.138 | C.11 |
| 1.2196 | B.139 | C.11 |
| 1.2197 | B.140 | C.11 |
| 1.2198 | B.141 | C.11 |
| 1.2199 | B.142 | C.11 |
| 1.2200 | B.143 | C.11 |
| 1.2201 | B.144 | C.11 |
| 1.2202 | B.145 | C.11 |
| 1.2203 | B.146 | C.11 |
| 1.2204 | B.147 | C.11 |
| 1.2205 | B.148 | C.11 |
| 1.2206 | B.149 | C.11 |
| 1.2207 | B.150 | C.11 |
| 1.2208 | B.151 | C.11 |
| 1.2209 | B.152 | C.11 |
| 1.2210 | B.153 | C.11 |
| 1.2211 | B.154 | C.11 |
| 1.2212 | B.155 | C.11 |
| 1.2213 | B.156 | C.11 |
| 1.2214 | B.157 | C.11 |
| 1.2215 | B.158 | C.11 |
| 1.2216 | B.159 | C.11 |
| 1.2217 | B.160 | C.11 |
| 1.2218 | B.161 | C.11 |
| 1.2219 | B.162 | C.11 |
| 1.2220 | B.163 | C.11 |
| 1.2221 | B.164 | C.11 |
| 1.2222 | B.165 | C.11 |
| 1.2223 | B.166 | C.11 |
| 1.2224 | B.167 | C.11 |
| 1.2225 | B.168 | C.11 |
| 1.2226 | B.169 | C.11 |
| 1.2227 | B.170 | C.11 |
| 1.2228 | B.171 | C.11 |
| 1.2229 | B.172 | C.11 |
| 1.2230 | B.173 | C.11 |
| 1.2231 | B.174 | C.11 |
| 1.2232 | B.175 | C.11 |
| 1.2233 | B.176 | C.11 |
| 1.2234 | B.177 | C.11 |
| 1.2235 | B.178 | C.11 |
| 1.2236 | B.179 | C.11 |
| 1.2237 | B.180 | C.11 |
| 1.2238 | B.181 | C.11 |
| 1.2239 | B.182 | C.11 |
| 1.2240 | B.183 | C.11 |
| 1.2241 | B.184 | C.11 |
| 1.2242 | B.185 | C.11 |
| 1.2243 | B.186 | C.11 |
| 1.2244 | B.187 | C.11 |
| 1.2245 | B.1 | C.12 |
| 1.2246 | B.2 | C.12 |
| 1.2247 | B.3 | C.12 |
| 1.2248 | B.4 | C.12 |
| 1.2249 | B.5 | C.12 |
| 1.2250 | B.6 | C.12 |
| 1.2251 | B.7 | C.12 |
| 1.2252 | B.8 | C.12 |
| 1.2253 | B.9 | C.12 |
| 1.2254 | B.10 | C.12 |
| 1.2255 | B.11 | C.12 |
| 1.2256 | B.12 | C.12 |
| 1.2257 | B.13 | C.12 |
| 1.2258 | B.14 | C.12 |
| 1.2259 | B.15 | C.12 |
| 1.2260 | B.16 | C.12 |
| 1.2261 | B.17 | C.12 |
| 1.2262 | B.18 | C.12 |
| 1.2263 | B.19 | C.12 |
| 1.2264 | B.20 | C.12 |
| 1.2265 | B.21 | C.12 |
| 1.2266 | B.22 | C.12 |
| 1.2267 | B.23 | C.12 |
| 1.2268 | B.24 | C.12 |
| 1.2269 | B.25 | C.12 |
| 1.2270 | B.26 | C.12 |
| 1.2271 | B.27 | C.12 |
| 1.2272 | B.28 | C.12 |
| 1.2273 | B.29 | C.12 |
| 1.2274 | B.30 | C.12 |
| 1.2275 | B.31 | C.12 |
| 1.2276 | B.32 | C.12 |
| 1.2277 | B.33 | C.12 |
| 1.2278 | B.34 | C.12 |
| 1.2279 | B.35 | C.12 |
| 1.2280 | B.36 | C.12 |
| 1.2281 | B.37 | C.12 |
| 1.2282 | B.38 | C.12 |
| 1.2283 | B.39 | C.12 |
| 1.2284 | B.40 | C.12 |
| 1.2285 | B.41 | C.12 |
| 1.2286 | B.42 | C.12 |
| 1.2287 | B.43 | C.12 |
| 1.2288 | B.44 | C.12 |
| 1.2289 | B.45 | C.12 |
| 1.2290 | B.46 | C.12 |
| 1.2291 | B.47 | C.12 |
| 1.2292 | B.48 | C.12 |
| 1.2293 | B.49 | C.12 |
| 1.2294 | B.50 | C.12 |
| 1.2295 | B.51 | C.12 |
| 1.2296 | B.52 | C.12 |
| 1.2297 | B.53 | C.12 |
| 1.2298 | B.54 | C.12 |
| 1.2299 | B.55 | C.12 |
| 1.2300 | B.56 | C.12 |
| 1.2301 | B.57 | C.12 |
| 1.2302 | B.58. | C.12 |
| 1.2303 | B.59 | C.12 |
| 1.2304 | B.60 | C.12 |
| 1.2305 | B.61 | C.12 |
| 1.2306 | B.62 | C.12 |
| 1.2307 | B.63 | C.12 |
| 1.2308 | B.64 | C.12 |
| 1.2309 | B.65 | C.12 |
| 1.2310 | B.66 | C.12 |
| 1.2311 | B.67 | C.12 |
| 1.2312 | B.68 | C.12 |
| 1.2313 | B.69 | C.12 |
| 1.2314 | B.70 | C.12 |
| 1.2315 | B.71 | C.12 |
| 1.2316 | B.72 | C.12 |
| 1.2317 | B.73 | C.12 |
| 1.2318 | B.74 | C.12 |
| 1.2319 | B.75 | C.12 |
| 1.2320 | B.76 | C.12 |
| 1.2321 | B.77 | C.12 |
| 1.2322 | B.78 | C.12 |
| 1.2323 | B.79 | C.12 |
| 1.2324 | B.80 | C.12 |
| 1.2325 | B.81 | C.12 |
| 1.2326 | B.82 | C.12 |
| 1.2327 | B.83 | C.12 |
| 1.2328 | B.84 | C.12 |
| 1.2329 | B.85 | C.12 |
| 1.2330 | B.86 | C.12 |
| 1.2331 | B.87 | C.12 |
| 1.2332 | B.88 | C.12 |
| 1.2333 | B.89 | C.12 |
| 1.2334 | B.90 | C.12 |
| 1.2335 | B.91 | C.12 |
| 1.2336 | B.92 | C.12 |
| 1.2337 | B.93 | C.12 |
| 1.2338 | B.94 | C.12 |
| 1.2339 | B.95 | C.12 |
| 1.2340 | B.96 | C.12 |
| 1.2341 | B.97 | C.12 |
| 1.2342 | B.98 | C.12 |
| 1.2343 | B.99 | C.12 |
| 1.2344 | B.100 | C.12 |
| 1.2345 | B.101 | C.12 |
| 1.2346 | B.102 | C.12 |
| 1.2347 | B.103 | C.12 |
| 1.2348 | B.104 | C.12 |
| 1.2349 | B.105 | C.12 |
| 1.2350 | B.106 | C.12 |
| 1.2351 | B.107 | C.12 |
| 1.2352 | B.108 | C.12 |
| 1.2353 | B.109 | C.12 |
| 1.2354 | B.110 | C.12 |
| 1.2355 | B.111 | C.12 |
| 1.2356 | B.112 | C.12 |
| 1.2357 | B.113 | C.12 |
| 1.2358 | B.114 | C.12 |
| 1.2359 | B.115 | C.12 |
| 1.2360 | B.116 | C.12 |
| 1.2361 | B.117 | C.12 |
| 1.2362 | B.118 | C.12 |
| 1.2363 | B.119 | C.12 |
| 1.2364 | B.120 | C.12 |
| 1.2365 | B.121 | C.12 |
| 1.2366 | B.122 | C.12 |
| 1.2367 | B.123 | C.12 |
| 1.2368 | B.124 | C.12 |
| 1.2369 | B.125 | C.12 |
| 1.2370 | B.126 | C.12 |
| 1.2371 | B.127 | C.12 |
| 1.2372 | B.128 | C.12 |
| 1.2373 | B.129 | C.12 |
| 1.2374 | B.130 | C.12 |
| 1.2375 | B.131 | C.12 |
| 1.2376 | B.132 | C.12 |
| 1.2377 | B.133 | C.12 |
| 1.2378 | B.134 | C.12 |
| 1.2379 | B.135 | C.12 |
| 1.2380 | B.136 | C.12 |
| 1.2381 | B.137 | C.12 |
| 1.2382 | B.138 | C.12 |
| 1.2383 | B.139 | C.12 |
| 1.2384 | B.140 | C.12 |
| 1.2385 | B.141 | C.12 |
| 1.2386 | B.142 | C.12 |
| 1.2387 | B.143 | C.12 |
| 1.2388 | B.144 | C.12 |
| 1.2389 | B.145 | C.12 |
| 1.2390 | B.146 | C.12 |
| 1.2391 | B.147 | C.12 |
| 1.2392 | B.148 | C.12 |
| 1.2393 | B.149 | C.12 |
| 1.2394 | B.150 | C.12 |
| 1.2395 | B.151 | C.12 |
| 1.2396 | B.152 | C.12 |
| 1.2397 | B.153 | C.12 |
| 1.2398 | B.154 | C.12 |
| 1.2399 | B.155 | C.12 |
| 1.2400 | B.156 | C.12 |
| 1.2401 | B.157 | C.12 |
| 1.2402 | B.158 | C.12 |
| 1.2403 | B.159 | C.12 |
| 1.2404 | B.160 | C.12 |
| 1.2405 | B.161 | C.12 |
| 1.2406 | B.162 | C.12 |
| 1.2407 | B.163 | C.12 |
| 1.2408 | B.164 | C.12 |
| 1.2409 | B.165 | C.12 |
| 1.2410 | B.166 | C.12 |
| 1.2411 | B.167 | C.12 |
| 1.2412 | B.168 | C.12 |
| 1.2413 | B.169 | C.12 |
| 1.2414 | B.170 | C.12 |
| 1.2415 | B.171 | C.12 |
| 1.2416 | B.172 | C.12 |
| 1.2417 | B.173 | C.12 |
| 1.2418 | B.174 | C.12 |
| 1.2419 | B.175 | C.12 |
| 1.2420 | B.176 | C.12 |
| 1.2421 | B.177 | C.12 |
| 1.2422 | B.178 | C.12 |
| 1.2423 | B.179 | C.12 |
| 1.2424 | B.180 | C.12 |
| 1.2425 | B.181 | C.12 |
| 1.2426 | B.182 | C.12 |
| 1.2427 | B.183 | C.12 |
| 1.2428 | B.184 | C.12 |
| 1.2429 | B.185 | C.12 |
| 1.2430 | B.186 | C.12 |
| 1.2431 | B.187 | C.12 |
| 1.2432 | B.1 | C.13 |
| 1.2433 | B.2 | C.13 |
| 1.2434 | B.3 | C.13 |
| 1.2435 | B.4 | C.13 |
| 1.2436 | B.5 | C.13 |
| 1.2437 | B.6 | C.13 |
| 1.2438 | B.7 | C.13 |
| 1.2439 | B.8 | C.13 |
| 1.2440 | B.9 | C.13 |
| 1.2441 | B.10 | C.13 |
| 1.2442 | B.11 | C.13 |
| 1.2443 | B.12 | C.13 |
| 1.2444 | B.13 | C.13 |
| 1.2445 | B.14 | C.13 |
| 1.2446 | B.15 | C.13 |
| 1.2447 | B.16 | C.13 |
| 1.2448 | B.17 | C.13 |
| 1.2449 | B.18 | C.13 |
| 1.2450 | B.19 | C.13 |
| 1.2451 | B.20 | C.13 |
| 1.2452 | B.21 | C.13 |
| 1.2453 | B.22 | C.13 |
| 1.2454 | B.23 | C.13 |
| 1.2455 | B.24 | C.13 |
| 1.2456 | B.25 | C.13 |
| 1.2457 | B.26 | C.13 |
| 1.2458 | B.27 | C.13 |
| 1.2459 | B.28 | C.13 |
| 1.2460 | B.29 | C.13 |
| 1.2461 | B.30 | C.13 |
| 1.2462 | B.31 | C.13 |
| 1.2463 | B.32 | C.13 |
| 1.2464 | B.33 | C.13 |
| 1.2465 | B.34 | C.13 |
| 1.2466 | B.35 | C.13 |
| 1.2467 | B.36 | C.13 |
| 1.2468 | B.37 | C.13 |
| 1.2469 | B.38 | C.13 |
| 1.2470 | B.39 | C.13 |
| 1.2471 | B.40 | C.13 |
| 1.2472 | B.41 | C.13 |
| 1.2473 | B.42 | C.13 |
| 1.2474 | B.43 | C.13 |
| 1.2475 | B.44 | C.13 |
| 1.2476 | B.45 | C.13 |
| 1.2477 | B.46 | C.13 |
| 1.2478 | B.47 | C.13 |
| 1.2479 | B.48 | C.13 |
| 1.2480 | B.49 | C.13 |
| 1.2481 | B.50 | C.13 |
| 1.2482 | B.51 | C.13 |
| 1.2483 | B.52 | C.13 |
| 1.2484 | B.53 | C.13 |
| 1.2485 | B.54 | C.13 |
| 1.2486 | B.55 | C.13 |
| 1.2487 | B.56 | C.13 |
| 1.2488 | B.57 | C.13 |
| 1.2489 | B.58. | C.13 |
| 1.2490 | B.59 | C.13 |
| 1.2491 | B.60 | C.13 |
| 1.2492 | B.61 | C.13 |
| 1.2493 | B.62 | C.13 |
| 1.2494 | B.63 | C.13 |
| 1.2495 | B.64 | C.13 |
| 1.2496 | B.65 | C.13 |
| 1.2497 | B.66 | C.13 |
| 1.2498 | B.67 | C.13 |
| 1.2499 | B.68 | C.13 |
| 1.2500 | B.69 | C.13 |
| 1.2501 | B.70 | C.13 |
| 1.2502 | B.71 | C.13 |
| 1.2503 | B.72 | C.13 |
| 1.2504 | B.73 | C.13 |
| 1.2505 | B.74 | C.13 |
| 1.2506 | B.75 | C.13 |
| 1.2507 | B.76 | C.13 |
| 1.2508 | B.77 | C.13 |
| 1.2509 | B.78 | C.13 |
| 1.2510 | B.79 | C.13 |
| 1.2511 | B.80 | C.13 |
| 1.2512 | B.81 | C.13 |
| 1.2513 | B.82 | C.13 |
| 1.2514 | B.83 | C.13 |
| 1.2515 | B.84 | C.13 |
| 1.2516 | B.85 | C.13 |
| 1.2517 | B.86 | C.13 |
| 1.2518 | B.87 | C.13 |
| 1.2519 | B.88 | C.13 |
| 1.2520 | B.89 | C.13 |
| 1.2521 | B.90 | C.13 |
| 1.2522 | B.91 | C.13 |
| 1.2523 | B.92 | C.13 |
| 1.2524 | B.93 | C.13 |
| 1.2525 | B.94 | C.13 |
| 1.2526 | B.95 | C.13 |
| 1.2527 | B.96 | C.13 |
| 1.2528 | B.97 | C.13 |
| 1.2529 | B.98 | C.13 |
| 1.2530 | B.99 | C.13 |
| 1.2531 | B.100 | C.13 |
| 1.2532 | B.101 | C.13 |
| 1.2533 | B.102 | C.13 |
| 1.2534 | B.103 | C.13 |
| 1.2535 | B.104 | C.13 |
| 1.2536 | B.105 | C.13 |
| 1.2537 | B.106 | C.13 |
| 1.2538 | B.107 | C.13 |
| 1.2539 | B.108 | C.13 |
| 1.2540 | B.109 | C.13 |
| 1.2541 | B.110 | C.13 |
| 1.2542 | B.111 | C.13 |
| 1.2543 | B.112 | C.13 |
| 1.2544 | B.113 | C.13 |
| 1.2545 | B.114 | C.13 |
| 1.2546 | B.115 | C.13 |
| 1.2547 | B.116 | C.13 |
| 1.2548 | B.117 | C.13 |
| 1.2549 | B.118 | C.13 |
| 1.2550 | B.119 | C.13 |
| 1.2551 | B.120 | C.13 |
| 1.2552 | B.121 | C.13 |
| 1.2553 | B.122 | C.13 |
| 1.2554 | B.123 | C.13 |
| 1.2555 | B.124 | C.13 |
| 1.2556 | B.125 | C.13 |
| 1.2557 | B.126 | C.13 |
| 1.2558 | B.127 | C.13 |
| 1.2559 | B.128 | C.13 |
| 1.2560 | B.129 | C.13 |
| 1.2561 | B.130 | C.13 |
| 1.2562 | B.131 | C.13 |
| 1.2563 | B.132 | C.13 |
| 1.2564 | B.133 | C.13 |
| 1.2565 | B.134 | C.13 |
| 1.2566 | B.135 | C.13 |
| 1.2567 | B.136 | C.13 |
| 1.2568 | B.137 | C.13 |
| 1.2569 | B.138 | C.13 |
| 1.2570 | B.139 | C.13 |
| 1.2571 | B.140 | C.13 |
| 1.2572 | B.141 | C.13 |
| 1.2573 | B.142 | C.13 |
| 1.2574 | B.143 | C.13 |
| 1.2575 | B.144 | C.13 |
| 1.2576 | B.145 | C.13 |
| 1.2577 | B.146 | C.13 |
| 1.2578 | B.147 | C.13 |
| 1.2579 | B.148 | C.13 |
| 1.2580 | B.149 | C.13 |
| 1.2581 | B.150 | C.13 |
| 1.2582 | B.151 | C.13 |
| 1.2583 | B.152 | C.13 |
| 1.2584 | B.153 | C.13 |
| 1.2585 | B.154 | C.13 |
| 1.2586 | B.155 | C.13 |
| 1.2587 | B.156 | C.13 |
| 1.2588 | B.157 | C.13 |
| 1.2589 | B.158 | C.13 |
| 1.2590 | B.159 | C.13 |
| 1.2591 | B.160 | C.13 |
| 1.2592 | B.161 | C.13 |
| 1.2593 | B.162 | C.13 |
| 1.2594 | B.163 | C.13 |
| 1.2595 | B.164 | C.13 |
| 1.2596 | B.165 | C.13 |
| 1.2597 | B.166 | C.13 |
| 1.2598 | B.167 | C.13 |
| 1.2599 | B.168 | C.13 |
| 1.2600 | B.169 | C.13 |
| 1.2601 | B.170 | C.13 |
| 1.2602 | B.171 | C.13 |
| 1.2603 | B.172 | C.13 |
| 1.2604 | B.173 | C.13 |
| 1.2605 | B.174 | C.13 |
| 1.2606 | B.175 | C.13 |
| 1.2607 | B.176 | C.13 |
| 1.2608 | B.177 | C.13 |
| 1.2609 | B.178 | C.13 |
| 1.2610 | B.179 | C.13 |
| 1.2611 | B.180 | C.13 |
| 1.2612 | B.181 | C.13 |
| 1.2613 | B.182 | C.13 |
| 1.2614 | B.183 | C.13 |
| 1.2615 | B.184 | C.13 |
| 1.2616 | B.185 | C.13 |
| 1.2617 | B.186 | C.13 |
| 1.2618 | B.187 | C.13 |
| 1.2619 | B.1 | C.14 |
| 1.2620 | B.2 | C.14 |
| 1.2621 | B.3 | C.14 |
| 1.2622 | B.4 | C.14 |
| 1.2623 | B.5 | C.14 |
| 1.2624 | B.6 | C.14 |
| 1.2625 | B.7 | C.14 |
| 1.2626 | B.8 | C.14 |
| 1.2627 | B.9 | C.14 |
| 1.2628 | B.10 | C.14 |
| 1.2629 | B.11 | C.14 |
| 1.2630 | B.12 | C.14 |
| 1.2631 | B.13 | C.14 |
| 1.2632 | B.14 | C.14 |
| 1.2633 | B.15 | C.14 |
| 1.2634 | B.16 | C.14 |
| 1.2635 | B.17 | C.14 |
| 1.2636 | B.18 | C.14 |
| 1.2637 | B.19 | C.14 |
| 1.2638 | B.20 | C.14 |
| 1.2639 | B.21 | C.14 |
| 1.2640 | B.22 | C.14 |
| 1.2641 | B.23 | C.14 |
| 1.2642 | B.24 | C.14 |
| 1.2643 | B.25 | C.14 |
| 1.2644 | B.26 | C.14 |
| 1.2645 | B.27 | C.14 |
| 1.2646 | B.28 | C.14 |
| 1.2647 | B.29 | C.14 |
| 1.2648 | B.30 | C.14 |
| 1.2649 | B.31 | C.14 |
| 1.2650 | B.32 | C.14 |
| 1.2651 | B.33 | C.14 |
| 1.2652 | B.34 | C.14 |
| 1.2653 | B.35 | C.14 |
| 1.2654 | B.36 | C.14 |
| 1.2655 | B.37 | C.14 |
| 1.2656 | B.38 | C.14 |
| 1.2657 | B.39 | C.14 |
| 1.2658 | B.40 | C.14 |
| 1.2659 | B.41 | C.14 |
| 1.2660 | B.42 | C.14 |
| 1.2661 | B.43 | C.14 |
| 1.2662 | B.44 | C.14 |
| 1.2663 | B.45 | C.14 |
| 1.2664 | B.46 | C.14 |
| 1.2665 | B.47 | C.14 |
| 1.2666 | B.48 | C.14 |
| 1.2667 | B.49 | C.14 |
| 1.2668 | B.50 | C.14 |
| 1.2669 | B.51 | C.14 |
| 1.2670 | B.52 | C.14 |
| 1.2671 | B.53 | C.14 |
| 1.2672 | B.54 | C.14 |
| 1.2673 | B.55 | C.14 |
| 1.2674 | B.56 | C.14 |
| 1.2675 | B.57 | C.14 |
| 1.2676 | B.58. | C.14 |
| 1.2677 | B.59 | C.14 |
| 1.2678 | B.60 | C.14 |
| 1.2679 | B.61 | C.14 |
| 1.2680 | B.62 | C.14 |
| 1.2681 | B.63 | C.14 |
| 1.2682 | B.64 | C.14 |
| 1.2683 | B.65 | C.14 |
| 1.2684 | B.66 | C.14 |
| 1.2685 | B.67 | C.14 |
| 1.2686 | B.68 | C.14 |
| 1.2687 | B.69 | C.14 |
| 1.2688 | B.70 | C.14 |
| 1.2689 | B.71 | C.14 |
| 1.2690 | B.72 | C.14 |
| 1.2691 | B.73 | C.14 |
| 1.2692 | B.74 | C.14 |
| 1.2693 | B.75 | C.14 |
| 1.2694 | B.76 | C.14 |
| 1.2695 | B.77 | C.14 |
| 1.2696 | B.78 | C.14 |
| 1.2697 | B.79 | C.14 |
| 1.2698 | B.80 | C.14 |
| 1.2699 | B.81 | C.14 |
| 1.2700 | B.82 | C.14 |
| 1.2701 | B.83 | C.14 |
| 1.2702 | B.84 | C.14 |
| 1.2703 | B.85 | C.14 |
| 1.2704 | B.86 | C.14 |
| 1.2705 | B.87 | C.14 |
| 1.2706 | B.88 | C.14 |
| 1.2707 | B.89 | C.14 |
| 1.2708 | B.90 | C.14 |
| 1.2709 | B.91 | C.14 |
| 1.2710 | B.92 | C.14 |
| 1.2711 | B.93 | C.14 |
| 1.2712 | B.94 | C.14 |
| 1.2713 | B.95 | C.14 |
| 1.2714 | B.96 | C.14 |
| 1.2715 | B.97 | C.14 |
| 1.2716 | B.98 | C.14 |
| 1.2717 | B.99 | C.14 |
| 1.2718 | B.100 | C.14 |
| 1.2719 | B.101 | C.14 |
| 1.2720 | B.102 | C.14 |
| 1.2721 | B.103 | C.14 |
| 1.2722 | B.104 | C.14 |
| 1.2723 | B.105 | C.14 |
| 1.2724 | B.106 | C.14 |
| 1.2725 | B.107 | C.14 |
| 1.2726 | B.108 | C.14 |
| 1.2727 | B.109 | C.14 |
| 1.2728 | B.110 | C.14 |
| 1.2729 | B.111 | C.14 |
| 1.2730 | B.112 | C.14 |
| 1.2731 | B.113 | C.14 |
| 1.2732 | B.114 | C.14 |
| 1.2733 | B.115 | C.14 |
| 1.2734 | B.116 | C.14 |
| 1.2735 | B.117 | C.14 |
| 1.2736 | B.118 | C.14 |
| 1.2737 | B.119 | C.14 |
| 1.2738 | B.120 | C.14 |
| 1.2739 | B.121 | C.14 |
| 1.2740 | B.122 | C.14 |
| 1.2741 | B.123 | C.14 |
| 1.2742 | B.124 | C.14 |
| 1.2743 | B.125 | C.14 |
| 1.2744 | B.126 | C.14 |
| 1.2745 | B.127 | C.14 |
| 1.2746 | B.128 | C.14 |
| 1.2747 | B.129 | C.14 |
| 1.2748 | B.130 | C.14 |
| 1.2749 | B.131 | C.14 |
| 1.2750 | B.132 | C.14 |
| 1.2751 | B.133 | C.14 |
| 1.2752 | B.134 | C.14 |
| 1.2753 | B.135 | C.14 |
| 1.2754 | B.136 | C.14 |
| 1.2755 | B.137 | C.14 |
| 1.2756 | B.138 | C.14 |
| 1.2757 | B.139 | C.14 |
| 1.2758 | B.140 | C.14 |
| 1.2759 | B.141 | C.14 |
| 1.2760 | B.142 | C.14 |
| 1.2761 | B.143 | C.14 |
| 1.2762 | B.144 | C.14 |
| 1.2763 | B.145 | C.14 |
| 1.2764 | B.146 | C.14 |
| 1.2765 | B.147 | C.14 |
| 1.2766 | B.148 | C.14 |
| 1.2767 | B.149 | C.14 |
| 1.2768 | B.150 | C.14 |
| 1.2769 | B.151 | C.14 |
| 1.2770 | B.152 | C.14 |
| 1.2771 | B.153 | C.14 |
| 1.2772 | B.154 | C.14 |
| 1.2773 | B.155 | C.14 |
| 1.2774 | B.156 | C.14 |
| 1.2775 | B.157 | C.14 |
| 1.2776 | B.158 | C.14 |
| 1.2777 | B.159 | C.14 |
| 1.2778 | B.160 | C.14 |
| 1.2779 | B.161 | C.14 |
| 1.2780 | B.162 | C.14 |
| 1.2781 | B.163 | C.14 |
| 1.2782 | B.164 | C.14 |
| 1.2783 | B.165 | C.14 |
| 1.2784 | B.166 | C.14 |
| 1.2785 | B.167 | C.14 |
| 1.2786 | B.168 | C.14 |
| 1.2787 | B.169 | C.14 |
| 1.2788 | B.170 | C.14 |
| 1.2789 | B.171 | C.14 |
| 1.2790 | B.172 | C.14 |
| 1.2791 | B.173 | C.14 |
| 1.2792 | B.174 | C.14 |
| 1.2793 | B.175 | C.14 |
| 1.2794 | B.176 | C.14 |
| 1.2795 | B.177 | C.14 |
| 1.2796 | B.178 | C.14 |
| 1.2797 | B.179 | C.14 |
| 1.2798 | B.180 | C.14 |
| 1.2799 | B.181 | C.14 |
| 1.2800 | B.182 | C.14 |
| 1.2801 | B.183 | C.14 |
| 1.2802 | B.184 | C.14 |
| 1.2803 | B.185 | C.14 |
| 1.2804 | B.186 | C.14 |
| 1.2805 | B.187 | C.14 |
| 1.2806 | B.1 | C.15 |
| 1.2807 | B.2 | C.15 |
| 1.2808 | B.3 | C.15 |
| 1.2809 | B.4 | C.15 |
| 1.2810 | B.5 | C.15 |
| 1.2811 | B.6 | C.15 |
| 1.2812 | B.7 | C.15 |
| 1.2813 | B.8 | C.15 |
| 1.2814 | B.9 | C.15 |
| 1.2815 | B.10 | C.15 |
| 1.2816 | B.11 | C.15 |
| 1.2817 | B.12 | C.15 |
| 1.2818 | B.13 | C.15 |
| 1.2819 | B.14 | C.15 |
| 1.2820 | B.15 | C.15 |
| 1.2821 | B.16 | C.15 |
| 1.2822 | B.17 | C.15 |
| 1.2823 | B.18 | C.15 |
| 1.2824 | B.19 | C.15 |
| 1.2825 | B.20 | C.15 |
| 1.2826 | B.21 | C.15 |
| 1.2827 | B.22 | C.15 |
| 1.2828 | B.23 | C.15 |
| 1.2829 | B.24 | C.15 |
| 1.2830 | B.25 | C.15 |
| 1.2831 | B.26 | C.15 |
| 1.2832 | B.27 | C.15 |
| 1.2833 | B.28 | C.15 |
| 1.2834 | B.29 | C.15 |
| 1.2835 | B.30 | C.15 |
| 1.2836 | B.31 | C.15 |
| 1.2837 | B.32 | C.15 |
| 1.2838 | B.33 | C.15 |
| 1.2839 | B.34 | C.15 |
| 1.2840 | B.35 | C.15 |
| 1.2841 | B.36 | C.15 |
| 1.2842 | B.37 | C.15 |
| 1.2843 | B.38 | C.15 |
| 1.2844 | B.39 | C.15 |
| 1.2845 | B.40 | C.15 |
| 1.2846 | B.41 | C.15 |
| 1.2847 | B.42 | C.15 |
| 1.2848 | B.43 | C.15 |
| 1.2849 | B.44 | C.15 |
| 1.2850 | B.45 | C.15 |
| 1.2851 | B.46 | C.15 |
| 1.2852 | B.47 | C.15 |
| 1.2853 | B.48 | C.15 |
| 1.2854 | B.49 | C.15 |
| 1.2855 | B.50 | C.15 |
| 1.2856 | B.51 | C.15 |
| 1.2857 | B.52 | C.15 |
| 1.2858 | B.53 | C.15 |
| 1.2859 | B.54 | C.15 |
| 1.2860 | B.55 | C.15 |
| 1.2861 | B.56 | C.15 |
| 1.2862 | B.57 | C.15 |
| 1.2863 | B.58. | C.15 |
| 1.2864 | B.59 | C.15 |
| 1.2865 | B.60 | C.15 |
| 1.2866 | B.61 | C.15 |
| 1.2867 | B.62 | C.15 |
| 1.2868 | B.63 | C.15 |
| 1.2869 | B.64 | C.15 |
| 1.2870 | B.65 | C.15 |
| 1.2871 | B.66 | C.15 |
| 1.2872 | B.67 | C.15 |
| 1.2873 | B.68 | C.15 |
| 1.2874 | B.69 | C.15 |
| 1.2875 | B.70 | C.15 |
| 1.2876 | B.71 | C.15 |
| 1.2877 | B.72 | C.15 |
| 1.2878 | B.73 | C.15 |
| 1.2879 | B.74 | C.15 |
| 1.2880 | B.75 | C.15 |
| 1.2881 | B.76 | C.15 |
| 1.2882 | B.77 | C.15 |
| 1.2883 | B.78 | C.15 |
| 1.2884 | B.79 | C.15 |
| 1.2885 | B.80 | C.15 |
| 1.2886 | B.81 | C.15 |
| 1.2887 | B.82 | C.15 |
| 1.2888 | B.83 | C.15 |
| 1.2889 | B.84 | C.15 |
| 1.2890 | B.85 | C.15 |
| 1.2891 | B.86 | C.15 |
| 1.2892 | B.87 | C.15 |
| 1.2893 | B.88 | C.15 |
| 1.2894 | B.89 | C.15 |
| 1.2895 | B.90 | C.15 |
| 1.2896 | B.91 | C.15 |
| 1.2897 | B.92 | C.15 |
| 1.2898 | B.93 | C.15 |
| 1.2899 | B.94 | C.15 |
| 1.2900 | B.95 | C.15 |
| 1.2901 | B.96 | C.15 |
| 1.2902 | B.97 | C.15 |
| 1.2903 | B.98 | C.15 |
| 1.2904 | B.99 | C.15 |
| 1.2905 | B.100 | C.15 |
| 1.2906 | B.101 | C.15 |
| 1.2907 | B.102 | C.15 |
| 1.2908 | B.103 | C.15 |
| 1.2909 | B.104 | C.15 |
| 1.2910 | B.105 | C.15 |
| 1.2911 | B.106 | C.15 |
| 1.2912 | B.107 | C.15 |
| 1.2913 | B.108 | C.15 |
| 1.2914 | B.109 | C.15 |
| 1.2915 | B.110 | C.15 |
| 1.2916 | B.111 | C.15 |
| 1.2917 | B.112 | C.15 |
| 1.2918 | B.113 | C.15 |
| 1.2919 | B.114 | C.15 |
| 1.2920 | B.115 | C.15 |
| 1.2921 | B.116 | C.15 |
| 1.2922 | B.117 | C.15 |
| 1.2923 | B.118 | C.15 |
| 1.2924 | B.119 | C.15 |
| 1.2925 | B.120 | C.15 |
| 1.2926 | B.121 | C.15 |
| 1.2927 | B.122 | C.15 |
| 1.2928 | B.123 | C.15 |
| 1.2929 | B.124 | C.15 |
| 1.2930 | B.125 | C.15 |
| 1.2931 | B.126 | C.15 |
| 1.2932 | B.127 | C.15 |
| 1.2933 | B.128 | C.15 |
| 1.2934 | B.129 | C.15 |
| 1.2935 | B.130 | C.15 |
| 1.2936 | B.131 | C.15 |
| 1.2937 | B.132 | C.15 |
| 1.2938 | B.133 | C.15 |
| 1.2939 | B.134 | C.15 |
| 1.2940 | B.135 | C.15 |
| 1.2941 | B.136 | C.15 |
| 1.2942 | B.137 | C.15 |
| 1.2943 | B.138 | C.15 |
| 1.2944 | B.139 | C.15 |
| 1.2945 | B.140 | C.15 |
| 1.2946 | B.141 | C.15 |
| 1.2947 | B.142 | C.15 |
| 1.2948 | B.143 | C.15 |
| 1.2949 | B.144 | C.15 |
| 1.2950 | B.145 | C.15 |
| 1.2951 | B.146 | C.15 |
| 1.2952 | B.147 | C.15 |
| 1.2953 | B.148 | C.15 |
| 1.2954 | B.149 | C.15 |
| 1.2955 | B.150 | C.15 |
| 1.2956 | B.151 | C.15 |
| 1.2957 | B.152 | C.15 |
| 1.2958 | B.153 | C.15 |
| 1.2959 | B.154 | C.15 |
| 1.2960 | B.155 | C.15 |
| 1.2961 | B.156 | C.15 |
| 1.2962 | B.157 | C.15 |
| 1.2963 | B.158 | C.15 |
| 1.2964 | B.159 | C.15 |
| 1.2965 | B.160 | C.15 |
| 1.2966 | B.161 | C.15 |
| 1.2967 | B.162 | C.15 |
| 1.2968 | B.163 | C.15 |
| 1.2969 | B.164 | C.15 |
| 1.2970 | B.165 | C.15 |
| 1.2971 | B.166 | C.15 |
| 1.2972 | B.167 | C.15 |
| 1.2973 | B.168 | C.15 |
| 1.2974 | B.169 | C.15 |
| 1.2975 | B.170 | C.15 |
| 1.2976 | B.171 | C.15 |
| 1.2977 | B.172 | C.15 |
| 1.2978 | B.173 | C.15 |
| 1.2979 | B.174 | C.15 |
| 1.2980 | B.175 | C.15 |
| 1.2981 | B.176 | C.15 |
| 1.2982 | B.177 | C.15 |
| 1.2983 | B.178 | C.15 |
| 1.2984 | B.179 | C.15 |
| 1.2985 | B.180 | C.15 |
| 1.2986 | B.181 | C.15 |
| 1.2987 | B.182 | C.15 |
| 1.2988 | B.183 | C.15 |
| 1.2989 | B.184 | C.15 |
| 1.2990 | B.185 | C.15 |
| 1.2991 | B.186 | C.15 |
| 1.2992 | B.187 | C.15 |
| 1.2993 | B.1 | C.16 |
| 1.2994 | B.2 | C.16 |
| 1.2995 | B.3 | C.16 |
| 1.2996 | B.4 | C.16 |
| 1.2997 | B.5 | C.16 |
| 1.2998 | B.6 | C.16 |
| 1.2999 | B.7 | C.16 |
| 1.3000 | B.8 | C.16 |
| 1.3001 | B.9 | C.16 |
| 1.3002 | B.10 | C.16 |
| 1.3003 | B.11 | C.16 |
| 1.3004 | B.12 | C.16 |
| 1.3005 | B.13 | C.16 |
| 1.3006 | B.14 | C.16 |
| 1.3007 | B.15 | C.16 |
| 1.3008 | B.16 | C.16 |
| 1.3009 | B.17 | C.16 |
| 1.3010 | B.18 | C.16 |
| 1.3011 | B.19 | C.16 |
| 1.3012 | B.20 | C.16 |
| 1.3013 | B.21 | C.16 |
| 1.3014 | B.22 | C.16 |
| 1.3015 | B.23 | C.16 |
| 1.3016 | B.24 | C.16 |
| 1.3017 | B.25 | C.16 |
| 1.3018 | B.26 | C.16 |
| 1.3019 | B.27 | C.16 |
| 1.3020 | B.28 | C.16 |
| 1.3021 | B.29 | C.16 |
| 1.3022 | B.30 | C.16 |
| 1.3023 | B.31 | C.16 |
| 1.3024 | B.32 | C.16 |
| 1.3025 | B.33 | C.16 |
| 1.3026 | B.34 | C.16 |
| 1.3027 | B.35 | C.16 |
| 1.3028 | B.36 | C.16 |
| 1.3029 | B.37 | C.16 |
| 1.3030 | B.38 | C.16 |
| 1.3031 | B.39 | C.16 |
| 1.3032 | B.40 | C.16 |
| 1.3033 | B.41 | C.16 |
| 1.3034 | B.42 | C.16 |
| 1.3035 | B.43 | C.16 |
| 1.3036 | B.44 | C.16 |
| 1.3037 | B.45 | C.16 |
| 1.3038 | B.46 | C.16 |
| 1.3039 | B.47 | C.16 |
| 1.3040 | B.48 | C.16 |
| 1.3041 | B.49 | C.16 |
| 1.3042 | B.50 | C.16 |
| 1.3043 | B.51 | C.16 |
| 1.3044 | B.52 | C.16 |
| 1.3045 | B.53 | C.16 |
| 1.3046 | B.54 | C.16 |
| 1.3047 | B.55 | C.16 |
| 1.3048 | B.56 | C.16 |
| 1.3049 | B.57 | C.16 |
| 1.3050 | B.58. | C.16 |
| 1.3051 | B.59 | C.16 |
| 1.3052 | B.60 | C.16 |
| 1.3053 | B.61 | C.16 |
| 1.3054 | B.62 | C.16 |
| 1.3055 | B.63 | C.16 |
| 1.3056 | B.64 | C.16 |
| 1.3057 | B.65 | C.16 |
| 1.3058 | B.66 | C.16 |
| 1.3059 | B.67 | C.16 |
| 1.3060 | B.68 | C.16 |
| 1.3061 | B.69 | C.16 |
| 1.3062 | B.70 | C.16 |
| 1.3063 | B.71 | C.16 |
| 1.3064 | B.72 | C.16 |
| 1.3065 | B.73 | C.16 |
| 1.3066 | B.74 | C.16 |
| 1.3067 | B.75 | C.16 |
| 1.3068 | B.76 | C.16 |
| 1.3069 | B.77 | C.16 |
| 1.3070 | B.78 | C.16 |
| 1.3071 | B.79 | C.16 |
| 1.3072 | B.80 | C.16 |
| 1.3073 | B.81 | C.16 |
| 1.3074 | B.82 | C.16 |
| 1.3075 | B.83 | C.16 |
| 1.3076 | B.84 | C.16 |
| 1.3077 | B.85 | C.16 |
| 1.3078 | B.86 | C.16 |
| 1.3079 | B.87 | C.16 |
| 1.3080 | B.88 | C.16 |
| 1.3081 | B.89 | C.16 |
| 1.3082 | B.90 | C.16 |
| 1.3083 | B.91 | C.16 |
| 1.3084 | B.92 | C.16 |
| 1.3085 | B.93 | C.16 |
| 1.3086 | B.94 | C.16 |
| 1.3087 | B.95 | C.16 |
| 1.3088 | B.96 | C.16 |
| 1.3089 | B.97 | C.16 |
| 1.3090 | B.98 | C.16 |
| 1.3091 | B.99 | C.16 |
| 1.3092 | B.100 | C.16 |
| 1.3093 | B.101 | C.16 |
| 1.3094 | B.102 | C.16 |
| 1.3095 | B.103 | C.16 |
| 1.3096 | B.104 | C.16 |
| 1.3097 | B.105 | C.16 |
| 1.3098 | B.106 | C.16 |
| 1.3099 | B.107 | C.16 |
| 1.3100 | B.108 | C.16 |
| 1.3101 | B.109 | C.16 |
| 1.3102 | B.110 | C.16 |
| 1.3103 | B.111 | C.16 |
| 1.3104 | B.112 | C.16 |
| 1.3105 | B.113 | C.16 |
| 1.3106 | B.114 | C.16 |
| 1.3107 | B.115 | C.16 |
| 1.3108 | B.116 | C.16 |
| 1.3109 | B.117 | C.16 |
| 1.3110 | B.118 | C.16 |
| 1.3111 | B.119 | C.16 |
| 1.3112 | B.120 | C.16 |
| 1.3113 | B.121 | C.16 |
| 1.3114 | B.122 | C.16 |
| 1.3115 | B.123 | C.16 |
| 1.3116 | B.124 | C.16 |
| 1.3117 | B.125 | C.16 |
| 1.3118 | B.126 | C.16 |
| 1.3119 | B.127 | C.16 |
| 1.3120 | B.128 | C.16 |
| 1.3121 | B.129 | C.16 |
| 1.3122 | B.130 | C.16 |
| 1.3123 | B.131 | C.16 |
| 1.3124 | B.132 | C.16 |
| 1.3125 | B.133 | C.16 |
| 1.3126 | B.134 | C.16 |
| 1.3127 | B.135 | C.16 |
| 1.3128 | B.136 | C.16 |
| 1.3129 | B.137 | C.16 |
| 1.3130 | B.138 | C.16 |
| 1.3131 | B.139 | C.16 |
| 1.3132 | B.140 | C.16 |
| 1.3133 | B.141 | C.16 |
| 1.3134 | B.142 | C.16 |
| 1.3135 | B.143 | C.16 |
| 1.3136 | B.144 | C.16 |
| 1.3137 | B.145 | C.16 |
| 1.3138 | B.146 | C.16 |
| 1.3139 | B.147 | C.16 |
| 1.3140 | B.148 | C.16 |
| 1.3141 | B.149 | C.16 |
| 1.3142 | B.150 | C.16 |
| 1.3143 | B.151 | C.16 |
| 1.3144 | B.152 | C.16 |
| 1.3145 | B.153 | C.16 |
| 1.3146 | B.154 | C.16 |
| 1.3147 | B.155 | C.16 |
| 1.3148 | B.156 | C.16 |
| 1.3149 | B.157 | C.16 |
| 1.3150 | B.158 | C.16 |
| 1.3151 | B.159 | C.16 |
| 1.3152 | B.160 | C.16 |
| 1.3153 | B.161 | C.16 |
| 1.3154 | B.162 | C.16 |
| 1.3155 | B.163 | C.16 |
| 1.3156 | B.164 | C.16 |
| 1.3157 | B.165 | C.16 |
| 1.3158 | B.166 | C.16 |
| 1.3159 | B.167 | C.16 |
| 1.3160 | B.168 | C.16 |
| 1.3161 | B.169 | C.16 |
| 1.3162 | B.170 | C.16 |
| 1.3163 | B.171 | C.16 |
| 1.3164 | B.172 | C.16 |
| 1.3165 | B.173 | C.16 |
| 1.3166 | B.174 | C.16 |
| 1.3167 | B.175 | C.16 |
| 1.3168 | B.176 | C.16 |
| 1.3169 | B.177 | C.16 |
| 1.3170 | B.178 | C.16 |
| 1.3171 | B.179 | C.16 |
| 1.3172 | B.180 | C.16 |
| 1.3173 | B.181 | C.16 |
| 1.3174 | B.182 | C.16 |
| 1.3175 | B.183 | C.16 |
| 1.3176 | B.184 | C.16 |
| 1.3177 | B.185 | C.16 |
| 1.3178 | B.186 | C.16 |
| 1.3179 | B.187 | C.16 |
| 1.3180 | B.1 | C.17 |
| 1.3181 | B.2 | C.17 |
| 1.3182 | B.3 | C.17 |
| 1.3183 | B.4 | C.17 |
| 1.3184 | B.5 | C.17 |
| 1.3185 | B.6 | C.17 |
| 1.3186 | B.7 | C.17 |
| 1.3187 | B.8 | C.17 |
| 1.3188 | B.9 | C.17 |
| 1.3189 | B.10 | C.17 |
| 1.3190 | B.11 | C.17 |
| 1.3191 | B.12 | C.17 |
| 1.3192 | B.13 | C.17 |
| 1.3193 | B.14 | C.17 |
| 1.3194 | B.15 | C.17 |
| 1.3195 | B.16 | C.17 |
| 1.3196 | B.17 | C.17 |
| 1.3197 | B.18 | C.17 |
| 1.3198 | B.19 | C.17 |
| 1.3199 | B.20 | C.17 |
| 1.3200 | B.21 | C.17 |
| 1.3201 | B.22 | C.17 |
| 1.3202 | B.23 | C.17 |
| 1.3203 | B.24 | C.17 |
| 1.3204 | B.25 | C.17 |
| 1.3205 | B.26 | C.17 |
| 1.3206 | B.27 | C.17 |
| 1.3207 | B.28 | C.17 |
| 1.3208 | B.29 | C.17 |
| 1.3209 | B.30 | C.17 |
| 1.3210 | B.31 | C.17 |
| 1.3211 | B.32 | C.17 |
| 1.3212 | B.33 | C.17 |
| 1.3213 | B.34 | C.17 |
| 1.3214 | B.35 | C.17 |
| 1.3215 | B.36 | C.17 |
| 1.3216 | B.37 | C.17 |
| 1.3217 | B.38 | C.17 |
| 1.3218 | B.39 | C.17 |
| 1.3219 | B.40 | C.17 |
| 1.3220 | B.41 | C.17 |
| 1.3221 | B.42 | C.17 |
| 1.3222 | B.43 | C.17 |
| 1.3223 | B.44 | C.17 |
| 1.3224 | B.45 | C.17 |
| 1.3225 | B.46 | C.17 |
| 1.3226 | B.47 | C.17 |
| 1.3227 | B.48 | C.17 |
| 1.3228 | B.49 | C.17 |
| 1.3229 | B.50 | C.17 |
| 1.3230 | B.51 | C.17 |
| 1.3231 | B.52 | C.17 |
| 1.3232 | B.53 | C.17 |
| 1.3233 | B.54 | C.17 |
| 1.3234 | B.55 | C.17 |
| 1.3235 | B.56 | C.17 |
| 1.3236 | B.57 | C.17 |
| 1.3237 | B.58. | C.17 |
| 1.3238 | B.59 | C.17 |
| 1.3239 | B.60 | C.17 |
| 1.3240 | B.61 | C.17 |
| 1.3241 | B.62 | C.17 |
| 1.3242 | B.63 | C.17 |
| 1.3243 | B.64 | C.17 |
| 1.3244 | B.65 | C.17 |
| 1.3245 | B.66 | C.17 |
| 1.3246 | B.67 | C.17 |
| 1.3247 | B.68 | C.17 |
| 1.3248 | B.69 | C.17 |
| 1.3249 | B.70 | C.17 |
| 1.3250 | B.71 | C.17 |
| 1.3251 | B.72 | C.17 |
| 1.3252 | B.73 | C.17 |
| 1.3253 | B.74 | C.17 |
| 1.3254 | B.75 | C.17 |
| 1.3255 | B.76 | C.17 |
| 1.3256 | B.77 | C.17 |
| 1.3257 | B.78 | C.17 |
| 1.3258 | B.79 | C.17 |
| 1.3259 | B.80 | C.17 |
| 1.3260 | B.81 | C.17 |
| 1.3261 | B.82 | C.17 |
| 1.3262 | B.83 | C.17 |
| 1.3263 | B.84 | C.17 |
| 1.3264 | B.85 | C.17 |
| 1.3265 | B.86 | C.17 |
| 1.3266 | B.87 | C.17 |
| 1.3267 | B.88 | C.17 |
| 1.3268 | B.89 | C.17 |
| 1.3269 | B.90 | C.17 |
| 1.3270 | B.91 | C.17 |
| 1.3271 | B.92 | C.17 |
| 1.3272 | B.93 | C.17 |
| 1.3273 | B.94 | C.17 |
| 1.3274 | B.95 | C.17 |
| 1.3275 | B.96 | C.17 |
| 1.3276 | B.97 | C.17 |
| 1.3277 | B.98 | C.17 |
| 1.3278 | B.99 | C.17 |
| 1.3279 | B.100 | C.17 |
| 1.3280 | B.101 | C.17 |
| 1.3281 | B.102 | C.17 |
| 1.3282 | B.103 | C.17 |
| 1.3283 | B.104 | C.17 |
| 1.3284 | B.105 | C.17 |
| 1.3285 | B.106 | C.17 |
| 1.3286 | B.107 | C.17 |
| 1.3287 | B.108 | C.17 |
| 1.3288 | B.109 | C.17 |
| 1.3289 | B.110 | C.17 |
| 1.3290 | B.111 | C.17 |
| 1.3291 | B.112 | C.17 |
| 1.3292 | B.113 | C.17 |
| 1.3293 | B.114 | C.17 |
| 1.3294 | B.115 | C.17 |
| 1.3295 | B.116 | C.17 |
| 1.3296 | B.117 | C.17 |
| 1.3297 | B.118 | C.17 |
| 1.3298 | B.119 | C.17 |
| 1.3299 | B.120 | C.17 |
| 1.3300 | B.121 | C.17 |
| 1.3301 | B.122 | C.17 |
| 1.3302 | B.123 | C.17 |
| 1.3303 | B.124 | C.17 |
| 1.3304 | B.125 | C.17 |
| 1.3305 | B.126 | C.17 |
| 1.3306 | B.127 | C.17 |
| 1.3307 | B.128 | C.17 |
| 1.3308 | B.129 | C.17 |
| 1.3309 | B.130 | C.17 |
| 1.3310 | B.131 | C.17 |
| 1.3311 | B.132 | C.17 |
| 1.3312 | B.133 | C.17 |
| 1.3313 | B.134 | C.17 |
| 1.3314 | B.135 | C.17 |
| 1.3315 | B.136 | C.17 |
| 1.3316 | B.137 | C.17 |
| 1.3317 | B.138 | C.17 |
| 1.3318 | B.139 | C.17 |
| 1.3319 | B.140 | C.17 |
| 1.3320 | B.141 | C.17 |
| 1.3321 | B.142 | C.17 |
| 1.3322 | B.143 | C.17 |
| 1.3323 | B.144 | C.17 |
| 1.3324 | B.145 | C.17 |
| 1.3325 | B.146 | C.17 |
| 1.3326 | B.147 | C.17 |
| 1.3327 | B.148 | C.17 |
| 1.3328 | B.149 | C.17 |
| 1.3329 | B.150 | C.17 |
| 1.3330 | B.151 | C.17 |
| 1.3331 | B.152 | C.17 |
| 1.3332 | B.153 | C.17 |
| 1.3333 | B.154 | C.17 |
| 1.3334 | B.155 | C.17 |
| 1.3335 | B.156 | C.17 |
| 1.3336 | B.157 | C.17 |
| 1.3337 | B.158 | C.17 |
| 1.3338 | B.159 | C.17 |
| 1.3339 | B.160 | C.17 |
| 1.3340 | B.161 | C.17 |
| 1.3341 | B.162 | C.17 |
| 1.3342 | B.163 | C.17 |
| 1.3343 | B.164 | C.17 |
| 1.3344 | B.165 | C.17 |
| 1.3345 | B.166 | C.17 |
| 1.3346 | B.167 | C.17 |
| 1.3347 | B.168 | C.17 |
| 1.3348 | B.169 | C.17 |
| 1.3349 | B.170 | C.17 |
| 1.3350 | B.171 | C.17 |
| 1.3351 | B.172 | C.17 |
| 1.3352 | B.173 | C.17 |
| 1.3353 | B.174 | C.17 |
| 1.3354 | B.175 | C.17 |
| 1.3355 | B.176 | C.17 |
| 1.3356 | B.177 | C.17 |
| 1.3357 | B.178 | C.17 |
| 1.3358 | B.179 | C.17 |
| 1.3359 | B.180 | C.17 |
| 1.3360 | B.181 | C.17 |
| 1.3361 | B.182 | C.17 |
| 1.3362 | B.183 | C.17 |
| 1.3363 | B.184 | C.17 |
| 1.3364 | B.185 | C.17 |
| 1.3365 | B.186 | C.17 |
| 1.3366 | B.187 | C.17 |
| 1.3367 | — | C.1 |
| 1.3368 | — | C.2 |
| 1.3369 | — | C.3 |
| 1.3370 | — | C.4 |
| 1.3371 | — | C.5 |
| 1.3372 | — | C.6 |
| 1.3373 | — | C.7 |
| 1.3374 | — | C.8 |
| 1.3375 | — | C.9 |
| 1.3376 | — | C.10 |
| 1.3377 | — | C.11 |
| 1.3378 | — | C.12 |
| 1.3379 | — | C.13 |
| 1.3380 | — | C.14 |
| 1.3381 | — | C.15 |
| 1.3382 | — | C.16 |
| 1.3383 | — | C.17 |

The specific number for each single composition is deductible as follows:

Composition 1.777 for example comprises the compound I.a foramsulfuron (B.29) and cyprosulfamide (C.4) (see table 1, entry 1.777; as well as table B, entry B.29 and table C, entry C.4).

Composition 2.777 for example comprises the compound I.a (see the definition for compositions 2.1 to 2.3383 below), foramsulfuron (B.29) and cyprosulfamide (C.4) (see table 1, entry 1.777; as well as table B, entry B.29 and table C, entry C.4).

Composition 7.777 for example comprises imazapyr (B.35) (see the definition for compositions 7.1 to 7.3383 below), and the compound I.a, foramsulfuron (B.29) and cyprosulfamide (C.4) (see table 1, entry 1.777; as well as table B, entry B.29 and table C, entry C.4).

Also especially preferred are compositions 2.1. to 2.3383 which differ from the corresponding compositions 1.1 to 1.3383 only in that they comprise as the active compound A the compound I.a.

Also especially preferred are

compositions 3.1. to 3.3383 which differ from the corresponding compositions 1.1 to 1.3383 only in that they additionally comprise B.2 as further herbicide B.

Also especially preferred are compositions 4.1. to 4.3383 which differ from the corresponding compositions 1.1 to 1.3383 only in that they additionally comprise B.8 as further herbicide B.

Also especially preferred are compositions 5.1. to 5.3383 which differ from the corresponding compositions 1.1 to 1.3383 only in that they additionally comprise B.30 as further herbicide B.

Also especially preferred are compositions 6.1. to 6.3383 which differ from the corresponding compositions 1.1 to 1.3383 only in that they additionally comprise B.32 as further herbicide B.

Also especially preferred are compositions 7.1. to 7.3383 which differ from the corresponding compositions 1.1 to 1.3383 only in that they additionally comprise B.35 as further herbicide B.

Also especially preferred are compositions 8.1. to 8.3383 which differ from the corresponding compositions 1.1 to 1.3383 only in that they additionally comprise B.38 as further herbicide B.

Also especially preferred are compositions 9.1. to 9.3383 which differ from the corresponding compositions 1.1 to 1.3383 only in that they additionally comprise B.40 as further herbicide B.

Also especially preferred are compositions 10.1. to 10.3383 which differ from the corresponding compositions 1.1 to 1.3383 only in that they additionally comprise B.51 as further herbicide B.

Also especially preferred are compositions 11.1. to 11.3383 which differ from the corresponding compositions 1.1 to 1.3383 only in that they additionally comprise B.55 as further herbicide B.

Also especially preferred are compositions 12.1. to 12.3383 which differ from the corresponding compositions 1.1 to 1.3383 only in that they additionally comprise B.56 as further herbicide B.

Also especially preferred are compositions 13.1. to 13.3383 which differ from the corresponding compositions 1.1 to 1.3383 only in that they additionally comprise B.64 as further herbicide B.

Also especially preferred are compositions 14.1. to 14.3383 which differ from the corresponding compositions 1.1 to 1.3383 only in that they additionally comprise B.66 as further herbicide B.

Also especially preferred are compositions 15.1. to 15.3383 which differ from the corresponding compositions 1.1 to 1.3383 only in that they additionally comprise B.67 as further herbicide B.

Also especially preferred are compositions 16.1. to 16.3383 which differ from the corresponding compositions 1.1 to 1.3383 only in that they additionally comprise B.68 as further herbicide B.

Also especially preferred are compositions 17.1. to 17.3383 which differ from the corresponding compositions 1.1 to 1.3383 only in that they additionally comprise B.69 as further herbicide B.

Also especially preferred are compositions 18.1. to 18.3383 which differ from the corresponding compositions 1.1 to 1.3383 only in that they additionally comprise B.73 as further herbicide B.

Also especially preferred are compositions 19.1. to 19.3383 which differ from the corresponding compositions 1.1 to 1.3383 only in that they additionally comprise B.76 as further herbicide B.

Also especially preferred are compositions 20.1. to 20.3383 which differ from the corresponding compositions 1.1 to 1.3383 only in that they additionally comprise B.81 as further herbicide B.

Also especially preferred are compositions 21.1. to 21.3383 which differ from the corresponding compositions 1.1 to 1.3383 only in that they additionally comprise B.82 as further herbicide B.

Also especially preferred are compositions 22.1. to 22.3383 which differ from the corresponding compositions 1.1 to 1.3383 only in that they additionally comprise B.85 as further herbicide B.

Also especially preferred are compositions 23.1. to 23.3383 which differ from the corresponding compositions 1.1 to 1.3383 only in that they additionally comprise B.88 as further herbicide B.

Also especially preferred are compositions 24.1. to 24.3383 which differ from the corresponding compositions 1.1 to 1.3383 only in that they additionally comprise B.89 as further herbicide B.

Also especially preferred are compositions 25.1. to 25.3383 which differ from the corresponding compositions 1.1 to 1.3383 only in that they additionally comprise B.92 as further herbicide B.

Also especially preferred are compositions 26.1. to 26.3383 which differ from the corresponding compositions 1.1 to 1.3383 only in that they additionally comprise B.93 as further herbicide B.

Also especially preferred are compositions 27.1. to 27.3383 which differ from the corresponding compositions 1.1 to 1.3383 only in that they additionally comprise B.96 as further herbicide B.

Also especially preferred are compositions 28.1. to 28.3383 which differ from the corresponding compositions 1.1 to 1.3383 only in that they additionally comprise B.97 as further herbicide B.

Also especially preferred are compositions 29.1. to 29.3383 which differ from the corresponding compositions 1.1 to 1.3383 only in that they additionally comprise B.100 as further herbicide B.

Also especially preferred are compositions 30.1. to 30.3383 which differ from the corresponding compositions 1.1 to 1.3383 only in that they additionally comprise B.100 and B.67 as further herbicides B.

Also especially preferred are compositions 31.1. to 31.3383 which differ from the corresponding compositions 1.1 to 1.3383 only in that they additionally comprise B.100 and B.76 as further herbicides B.

Also especially preferred are compositions 32.1. to 32.3383 which differ from the corresponding compositions 1.1 to 1.3383 only in that they additionally comprise B.100 and B.82 as further herbicides B.

Also especially preferred are compositions 33.1. to 33.3383 which differ from the corresponding compositions 1.1 to 1.3383 only in that they additionally comprise B.101 as further herbicide B.

Also especially preferred are compositions 34.1. to 34.3383 which differ from the corresponding compositions 1.1 to 1.3383 only in that they additionally comprise B.101 and B.67 as further herbicides B.

Also especially preferred are compositions 35.1. to 35.3383 which differ from the corresponding compositions 1.1 to 1.3383 only in that they additionally comprise B.101 and B.76 as further herbicides B.

Also especially preferred are compositions 36.1. to 36.3383 which differ from the corresponding compositions 1.1 to 1.3383 only in that they additionally comprise B.101 and B.82 as further herbicides B.

Also especially preferred are compositions 37.1. to 37.3383 which differ from the corresponding compositions 1.1 to 1.3383 only in that they additionally comprise B.103 as further herbicide B.

Also especially preferred are compositions 38.1. to 38.3383 which differ from the corresponding compositions 1.1 to 1.3383 only in that they additionally comprise B.104 as further herbicide B.

Also especially preferred are compositions 39.1. to 39.3383 which differ from the corresponding compositions 1.1 to 1.3383 only in that they additionally comprise B. 104 and B.67 as further herbicides B.

Also especially preferred are compositions 40.1. to 40.3383 which differ from the corresponding compositions 1.1 to 1.3383 only in that they additionally comprise B. 104 and B.76 as further herbicides B.

Also especially preferred are compositions 41.1. to 41.3383 which differ from the corresponding compositions 1.1 to 1.3383 only in that they additionally comprise B. 104 and B.82 as further herbicides B.

Also especially preferred are compositions 42.1. to 42.3383 which differ from the corresponding compositions 1.1 to 1.3383 only in that they additionally comprise B.106 as further herbicide B.

Also especially preferred are compositions 43.1. to 43.3383 which differ from the corresponding compositions 1.1 to 1.3383 only in that they additionally comprise B.107 as further herbicide B.

Also especially preferred are compositions 44.1. to 44.3383 which differ from the corresponding compositions 1.1 to 1.3383 only in that they additionally comprise B.107 and B.67 as further herbicides B.

Also especially preferred are compositions 45.1. to 45.3383 which differ from the corresponding compositions 1.1 to 1.3383 only in that they additionally comprise B.107 and B.76 as further herbicides B.

Also especially preferred are compositions 46.1. to 46.3383 which differ from the corresponding compositions 1.1 to 1.3383 only in that they additionally comprise B.107 and B.82 as further herbicides B.

Also especially preferred are compositions 47.1. to 47.3383 which differ from the corresponding compositions 1.1 to 1.3383 only in that they additionally comprise B.112 as further herbicide B.

Also especially preferred are compositions 48.1. to 48.3383 which differ from the corresponding compositions 1.1 to 1.3383 only in that they additionally comprise B.112 and B.67 as further herbicides B.

Also especially preferred are compositions 49.1. to 49.3383 which differ from the corresponding compositions 1.1 to 1.3383 only in that they additionally comprise B.112 and B.92 as further herbicides B.

Also especially preferred are compositions 50.1. to 50.3383 which differ from the corresponding compositions 1.1 to 1.3383 only in that they additionally comprise B.112 and B.100 as further herbicides B.

Also especially preferred are compositions 51.1. to 51.3383 which differ from the corresponding compositions 1.1 to 1.3383 only in that they additionally comprise B.112 and B.124 as further herbicides B.

Also especially preferred are compositions 52.1. to 52.3383 which differ from the corresponding compositions 1.1 to 1.3383 only in that they additionally comprise B.112 and B.101 as further herbicides B.

Also especially preferred are compositions 53.1. to 53.3383 which differ from the corresponding compositions 1.1 to 1.3383 only in that they additionally comprise B.112 and B.104 as further herbicides B.

Also especially preferred are compositions 54.1. to 54.3383 which differ from the corresponding compositions 1.1 to 1.3383 only in that they additionally comprise B.112 and B.107 as further herbicides B.

Also especially preferred are compositions 55.1. to 55.3383 which differ from the corresponding compositions 1.1 to 1.3383 only in that they additionally comprise B.118 as further herbicide B.

Also especially preferred are compositions 56.1. to 56.3383 which differ from the corresponding compositions 1.1 to 1.3383 only in that they additionally comprise B.122 as further herbicide B.

Also especially preferred are compositions 57.1. to 57.3383 which differ from the corresponding compositions 1.1 to 1.3383 only in that they additionally comprise B.124 as further herbicide B.

Also especially preferred are compositions 58.1. to 58.3383 which differ from the corresponding compositions 1.1 to 1.3383 only in that they additionally comprise B.127 as further herbicide B.

Also especially preferred are compositions 59.1. to 59.3383 which differ from the corresponding compositions 1.1 to 1.3383 only in that they additionally comprise B.128 as further herbicide B.

Also especially preferred are compositions 60.1. to 60.3383 which differ from the corresponding compositions 1.1 to 1.3383 only in that they additionally comprise B.129 as further herbicide B.

Also especially preferred are compositions 61.1. to 61.3383 which differ from the corresponding compositions 1.1 to 1.3383 only in that they additionally comprise B.131 as further herbicide B.

Also especially preferred are compositions 62.1. to 62.3383 which differ from the corresponding compositions 1.1 to 1.3383 only in that they additionally comprise B.133 as further herbicide B.

Also especially preferred are compositions 63.1. to 63.3383 which differ from the corresponding compositions 11.1 to 1.3383 only in that they additionally comprise B.134 as further herbicide B.

Also especially preferred are compositions 64.1. to 64.3383 which differ from the corresponding compositions 1.1 to 1.3383 only in that they additionally comprise B.139 as further herbicide B.

Also especially preferred are compositions 65.1. to 65.3383 which differ from the corresponding compositions 1.1 to 1.3383 only in that they additionally comprise B.146 as further herbicide B.

Also especially preferred are compositions 66.1. to 66.3383 which differ from the corresponding compositions 1.1 to 1.3383 only in that they additionally comprise B.149 as further herbicide B.

Also especially preferred are compositions 67.1. to 67.3383 which differ from the corresponding compositions 1.1 to 1.3383 only in that they additionally comprise B.162 as further herbicide B.

Also especially preferred are compositions 68.1. to 68.3383 which differ from the corresponding compositions 1.1 to 1.3383 only in that they additionally comprise B.165 as further herbicide B.

Also especially preferred are compositions 69.1. to 69.3383 which differ from the corresponding compositions 1.1 to 1.3383 only in that they additionally comprise B.176 as further herbicide B.

Hereinbelow, the compounds of the formula I are illustrated by way of examples, without limiting the subject matter of the present invention to the examples shown.

### I. SYNTHESIS EXAMPLES

With appropriate modification of the starting materials, the procedures given in the synthesis examples below were used to obtain further compounds I. The compounds obtained in this manner are listed in the table that follows, together with physical data.

The products shown below were characterized by determination of the melting point, NMR spectroscopy or the masses ([m/z]) determined by HPLC-MS spectrometry.

HPLC-MS=high performance liquid chromatography coupled with mass spectrometry; HPLC column:

RP-18 column (Chromolith Speed ROD from Merck KgaA, Germany), 50*4.6 mm; mobile phase: acetonitrile+0.1% trifluoroacetic acid (TFA)/water+0.1% TFA, using a gradient from 5:95 to 100:0 over 5 minutes at 40° C., flow rate 1.8 ml/min.

MS: quadrupole electrospray ionization, 80 V (positive mode).

DBU: 1,8-Diazabicyclo[5.4.0]undec-7-ene

EtOAc: acetic acid ethyl ester

### Example 1

### Preparation of [7-(6-chloro-4-methyl-3-pyridyl)-6,6-dioxo-5H-thiopyrano[4,3-b]pyridin-8-yl]2,2-dimethylpropanoate (see compound I-14 in the Table I below)

### Step 1: 5-(Bromomethyl)-2-chloro-4-methyl-pyridine

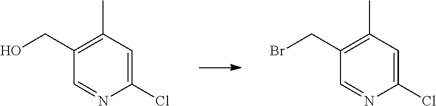

To a solution of (6-chloro-4-methyl-3-pyridyl)methanol (2.5 g, 16 mmol) in 100 mL anhydrous dichloromethane was added PBr₃ (6.38 g, 24 mmol) at 0° C. dropwise. After stirring for 1 h at 0° C., the reaction was carefully quenched by water dropwise at 0° C. Then 100 mL dichloromethane were added and the organic layer was washed with water twice, saturated NaHCO₃ solution twice and saturated NaCl solution once. It was dried over Na₂SO₄, and concentrated to afford the product (2.67 g, 76.7%, MS+1=220).

¹H NMR (CDCl₃ 400 MHz): δ 8.27 (s, 1H), 7.18 (s, 1H), 4.45 (s, 2H), 2.42 (s, 3H).

### Step 2: S-[(6-chloro-4-methyl-3-pyridyl)methyl]ethanethioate

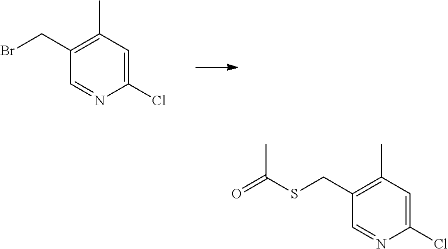

To a solution of 5-(bromomethyl)-2-chloro-4-methyl-pyridine (2.23 g, 10.2 mmol) in 50 mL acetone was added potassium thioacetate (1.74 g, 15.3 mmol). The mixture was stirred for 2.5 hours at room temperature. Then the solvent was evaporated and the residue purified by column to give the product (2.1 g, 95.8%, MS+1=216).

¹H NMR (CDCl₃ 400 MHz): δ 8.26 (s, 1H), 7.13 (s, 1H), 4.08 (s, 2H), 2.35 (s, 3H), 2.31 (s, 3H).

### Step 3: Methyl 3-[(6-chloro-4-methyl-3-pyridyl)methylsulfanylmethyl]pyridine-2-carboxylate

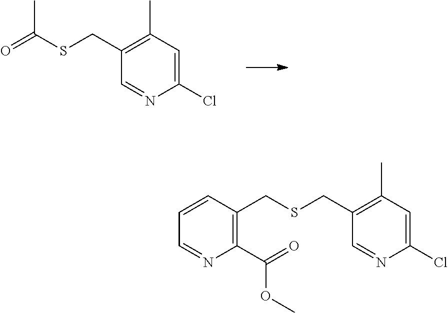 [SMILES: CC(=O)SCc1cnc(Cl)cc1C ; COC(=O)c1ncccc1CSCc1cnc(Cl)cc1C]

To a solution of S-[(6-chloro-4-methyl-3-pyridyl)methyl]ethanethioate (1.67 g, 7.8 mmol) in 50 mL methanol was added K₂CO₃ (2.14 g, 15.5 mmol). The mixture was stirred for 30 min at room temperature. Then methyl-(3-bromomethyl)pyridine-2-carboxylate (1.78 g, 7.8 mmol) was added and it was stirred for 2.5 hours at room temperature. The mixture was concentrated to give the crude product (2.87 g, crude, MS+1=322).

### Step 4: Methyl 3-[(6-chloro-4-methyl-3-pyridyl)methylsulfonylmethyl]pyridine-2-carboxylate

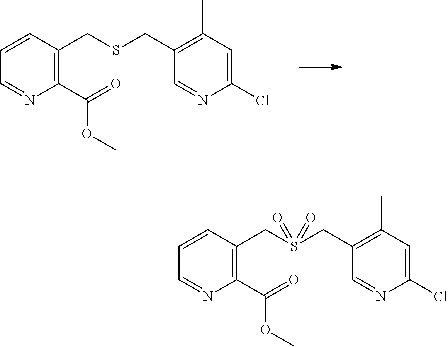 [SMILES: COC(=O)c1ncccc1CSCc1cnc(Cl)cc1C ; COC(=O)c1ncccc1CS(=O)(=O)Cc1cnc(Cl)cc1C]

To a solution of methyl 3-[(6-chloro-4-methyl-3-pyridyl)methylsulfanylmethyl]pyridine-2-carboxylate (2.87 g, 8.9 mmol) in 100 mL anhydrous dichloromethane was added m-chloroperbenzoic acid (3.85 g, 17.8 mmol) at room temperature. After stirring for 3 h at room temperature, the reaction was quenched by Na₂S₂O₃ in saturated NaHCO₃ solution. Then 100 mL dichloromethane were added and the organic layer was washed with saturated NaHCO₃ solution twice and saturated NaCl solution once. It was dried over Na₂SO₄, and concentrated to afford the product (2.67 g, 84.4%, MS+1=355).

¹H NMR (CDCl₃ 400 MHz): δ 8.80 (d, J=4.4 Hz, 1H), 8.23 (s, 1H), 7.79 (d, 1H, J=7.6 Hz), 7.57 (dd, J₁=4.8 Hz, J₂=8 Hz, 1H), 7.23 (s, 1H), 4.96 (s, 2H), 4.33 (s, 2H), 4.04 (s, 3H), 2.38 (s, 3H).

### Step 5: 7-(6-chloro-4-methyl-3-pyridyl)-6,6-dioxo-5H-thiopyrano[4,3-b]pyridin-8-ol

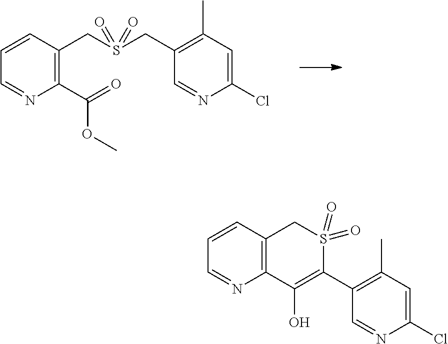 [SMILES: COC(=O)c1ncccc1CS(=O)(=O)Cc1cnc(Cl)cc1C ; Cc1cc(Cl)ncc1C1=C(O)c2ncccc2CS1(=O)=O]

To a solution of methyl 3-[(6-chloro-4-methyl-3-pyridyl)methylsulfonylmethyl]pyridine-2-carboxylate (1.98 g, 5.6 mmol) in 50 mL acetonitrile was added DBU (1.27 g, 8.4 mmol) and it was stirred at 60° C. overnight. Then the solvent was evaporated and water was added to the residue. The aqueous phase was acidified to pH 2, and it was extracted with EtOAc. The combined organic layer was dried and concentrated to give the crude product, which was purified by recrystallization with EtOAc to afford the product (1.5 g, 83.7%, MS+1=322).

¹H NMR (CDCl₃ 400 MHz): δ 8.98 (brs, 1H), 8.67 (d, J=4.8 Hz, 1H), 8.40 (s, 1H), 7.79 (d, J=8 Hz, 1H), 7.53 (dd, J₁=4.8 Hz, J₂=7.6 Hz, 1H), 7.32 (s, 1H), 4.60-4.70 (m, 2H), 2.39 (s, 3H).

### Step 6: [7-(6-chloro-4-methyl-3-pyridyl)-6,6-dioxo-5H-thiopyrano[4,3-b]pyridin-8-yl]2,2-dimethylpropanoate (=compound I-14 in Table I below)

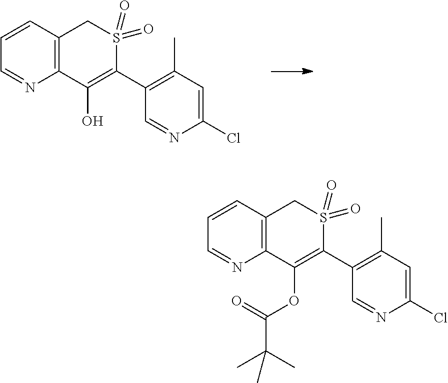 [SMILES: Cc1cc(Cl)ncc1C1=C(O)c2ncccc2CS1(=O)=O ; Cc1cc(Cl)ncc1C1=C(OC(=O)C(C)(C)C)c2ncccc2CS1(=O)=O]

To 7-(6-chloro-4-methyl-3-pyridyl)-6,6-dioxo-5H-thiopyrano[4,3-b]pyridin-8-ol (840 mg, 2.6 mmol) in 30 mL anhydrous toluene was added pivaloyl chloride (624 mg, 5.2 mmol) and pyridine (824 mg, 10.4 mmol). The mixture was stirred for 2 hrs at 90° C. Then the solvent was evaporated to give the crude product, which was purified by column (dichloromethane:methanol=100:1) to afford the product (1.1 g, 100%).

### Example 2

### Preparation of [7-(6-chloro-4-methyl-3-pyridyl)-5,5-dimethyl-6,6-dioxo-thiopyrano[4,3-b]pyridin-8-yl]2,2-dimethylpropanoate (see compound I-15 in the Table I below)

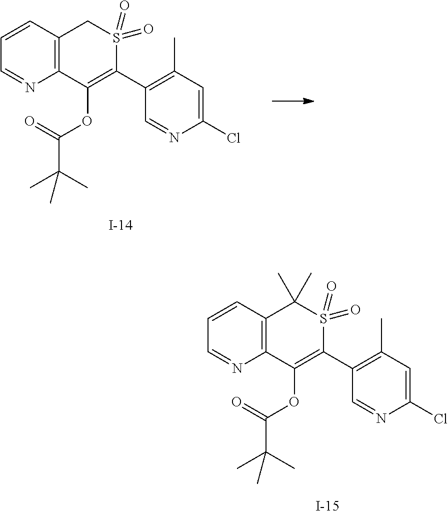

To a solution of [7-(6-chloro-4-methyl-3-pyridyl)-6,6-dioxo-5H-thiopyrano[4,3-b]pyridin-8-yl]2,2-dimethylpropanoate (897 mg, 2.2 mmol) in 30 mL dimethylformamide was added Cs₂CO₃ (2.16 g, 6.62 mmol). It was stirred for 30 min at room temperature, then Mel (1.25 g, 8.8 mmol) was added and it was stirred overnight at room temperature. 150 mL H₂O was added and the mixture extracted with EtOAc twice. The combined organic layer was dried and concentrated to give the crude product, which was purified by column (dichloromethane:methanol=100:1) to give the product (960 mg, 100%).

### Example 3

### Preparation of 7-(6-chloro-4-methyl-3-pyridyl)-5,5-dimethyl-6,6-dioxo-thiopyrano[4,3-b]pyridin-8-ol (see compound I-13 in the Table I below)

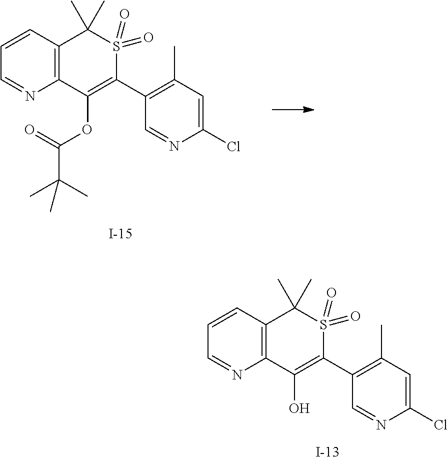

To a mixture of [7-(6-chloro-4-methyl-3-pyridyl)-5,5-dimethyl-6,6-dioxo-thiopyrano[4,3-b]pyridin-8-yl]2,2-dimethylpropanoate (810 mg, 1.9 mmol) in 8 mL tetrahydrofurane and 4 mL H₂O was added LiOH (313 mg, 7.5 mmol) and it's stirred for overnight at 60° C. Then water was added and the mixture acidified to pH 2. It was extracted with EtOAc twice. The combined organic layer was dried and concentrated to give the crude product, which was purified by preparative HPLC to give the product (390 mg, 59.7%).

¹H NMR (CDCl₃ 400 MHz): δ 8.60 (d, 1H, J=4.0 Hz), 8.35 (s, 1H), 7.89 (d, 1H, J=8 Hz), 7.54 (dd, J₁=4.8 Hz, J₂=8 Hz, 1H), 7.32 (s, 1H), 2.39 (s, 3H), 1.83 (s, 6H).

TABLE I
Compounds of the formula I.3

| | | | | | | | | LC- |
|---|---|---|---|---|---|---|---|---|
| | | | | | | | | MS |
| No. | R | R¹ | R² | R³ | R^{x} | R^{y} | A | (m/z) |
| I-1 | OPiv | Cl | H | Cl | H | H | CR² | 427.2 |
| I-2 | OPiv | Cl | H | Cl | CH₃ | CH₃ | CR² | 455.1 |
| I-3 | OH | Cl | H | Cl | H | H | CR² | 343.1 |
| I-4 | OH | Cl | H | Cl | CH₃ | CH₃ | CR² | 371.2 |
| I-5 | OH | CF₃ | H | OCH₃ | H | H | CR² | 373.2 |
| I-6 | OH | CF₃ | H | OCH₃ | CH₃ | CH₃ | CR² | 401.2 |
| I-7 | OH | CF₃ | H | Cl | H | H | CR² | 377.1 |
| I-8 | OH | CF₃ | H | Cl | CH₃ | CH₃ | CR² | 405.2 |
| I-9 | OH | CF₃ | — | NHCOCH₂—CH(CH₃)₂ | H | H | N | 443.1 |
| 1-10 | OH | Cl | H | Br | H | H | CR² | 386.9 |
| 1-11 | OH | CF₃ | — | NH₂ | H | H | N | 359.0 |
| 1-12 | OH | CH₃ | H | Cl | H | H | CR² | 323.2 |
| 1-13 | OH | CH₃ | H | Cl | CH₃ | CH₃ | CR² | 351.2 |
| 1-14 | OPiv | CH₃ | H | Cl | H | H | CR² | 407.1 |
| 1-15 | OPiv | CH₃ | H | Cl | CH₃ | CH₃ | CR² | 435.1 |
| 1-16 | OH | CF₃ | H | CH₃ | CH₃ | CH₃ | CR₂ | 385.1 |
| 1-17 | OH | CF₃ | H | CH₃ | H | H | CR² | 357.1 |
| 1-18 | OH | OCH₂CH₂—OCH₃ | H | CF₃ | CH₃ | CH₃ | CR² | 445.1 |
| 1-19 | OH | OCH₂CH₂—OCH₃ | H | CF₃ | H | H | CR² | 417.1 |
| I-20 | OH | SO₂H₃ | H | CF₃ | H | H | CR² | 421.0 |
| 1-21 | OH | SO₂H₃ | H | CF₃ | CH₃ | CH₃ | CR² | 449.0 |
| I-22 | OH | OCH₃ | H | CF₃ | H | H | CR² | 373.2 |
| I-23 | OH | OCH₃ | H | CF₃ | CH₃ | CH₃ | CR² | 401.2 |
| I-24 | OH | CF₃ | N(CH₃)—SO₂CH₃ | Cl | CH₃ | CH₃ | CR² | 512.0 |
| I-25 | OH | CF₃ | N(CH₃)—SO₂CH₃ | Cl | H | H | CR² | 484.0 |
| I-26 | OH | Cl | H | CF₃ | H | H | CR² | 377.0 |
| I-27 | OH | Cl | H | CF₃ | CH₃ | CH₃ | CR² | 405.1 |
| I-28 | OH | CH(CH₃)₂ | H | Cl | H | H | CR² | 351.1 |
| I-29 | OPiv | CH(CH₃)₂ | H | Cl | H | H | CR² | 435.3 |
| I-30 | OH | CH(CH₃)₂ | H | Cl | CH₃ | CH₃ | CR² | 379.2 |
| 1-31 | OPiv | CH(CH₃)₂ | H | Cl | CH₃ | CH₃ | CR² | 463.3 |
| I-32 | OH | CH₃ | — | O-c-Bu | H | H | N | 360.0 |
| I-33 | OH | CH₃ | — | O-c-Bu | CH₃ | CH₃ | N | 388.0 |
| I-34 | OH | CH₃ | — | OCH(CH₃)₂ | H | H | N | 348.1 |
| I-35 | OH | CH₃ | — | OCH(CH₃)₂ | CH₃ | CH₃ | N | 376.1 |
| I-36 | OH | CF₃ | — | OCH₃ | H | H | N | 374.0 |
| I-37 | OH | CF₃ | — | OCH₃ | CH₃ | CH₃ | N | 402.0 |
| I-38 | OH | CH₃ | — | N(CH₃)SO₂CH₃ | H | H | N | 397.0 |
| I-39 | OH | CH₃ | — | N(CH₃)SO₂CH₃ | CH₃ | CH₃ | N | 425.2 |
| I-40 | OH | CH₃ | — | NHSO₂CH₃ | H | H | N | 383.0 |
| 1-41 | OH | CH₃ | — | SCH₃ | H | H | N | 335.9 |
| I-42 | OH | CH₃ | — | SCH₃ | CH₃ | CH₃ | N | 364.0 |
| I-43 | OH | CH₃ | — | OCH₃ | H | H | N | 319.9 |
| I-44 | OH | CH₃ | — | OCH₃ | CH₃ | CH₃ | N | 348.0 |
| I-45 | OH | CF₃ | — | O-c-Bu | H | H | N | 414.0 |
| I-46 | OH | c-Pr | H | Cl | H | H | CR² | 349.1 |
| I-47 | OH | c-Pr | H | Cl | CH₃ | CH₃ | CR² | 377.0 |

Wherein OPiv denotes (CH₃)₃CC(O)O, c-Pr denotes cyclopropyl and c-Bu denotes cyclobutyl.

### II. USE EXAMPLES

The herbicidal activity of the compounds of formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer code | Scientific name | English name |
|---|---|---|
| ABUTH | Abutilon theophrasti | velvetleaf |
| AMARE | Amaranthus retroflexus | common amaranth |
| AVEFA | Avena fatua | wild oat |
| CHEAL | Chenopodium album | lampsquaters |
| IPOHE | Ipomoea hederacea | ivy-leaf morning glory |
| SETVI | Setaria viridis | green foxtail |

At an application rate of 0.125 kg/ha, the compound I-8, applied by the post-emergence method, showed very good herbicidal activity against ABUTH.

At an application rate of 0.125 kg/ha, the compounds I-2, I-4, I-5, I-6, I-7 and I-8, applied by the post-emergence method, showed very good herbicidal activity and the compound I-3 good herbicidal activity against AMARE.

At an application rate of 0.125 kg/ha, the compound I-5, applied by the post-emergence method, showed very good herbicidal activity against AVEFA.

At an application rate of 0.125 kg/ha, the compounds I-2, I-3, I-4, I-5, I-6 and I-7, applied by the post-emergence method, showed very good herbicidal activity against CHEAL.

At an application rate of 0.125 kg/ha, the compounds I-6 and I-8, applied by the post-emergence method, showed very good herbicidal activity against IPOHE.

At an application rate of 0.125 kg/ha, the compounds I-2, I-3, I-4 and I-7, applied by the post-emergence method, showed very good herbicidal activity against SETVI.

1-15. (canceled)

16: A compound of formula I 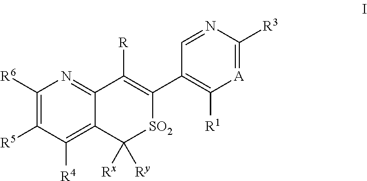
wherein
R is hydroxy or O—R^{A}, where R^{A} is C₁-C₈-alkyl, C₂-C₈-alkenyl, C₂-C₈-alkynyl, aryl-C₁-C₄-alkyl, C₁-C₈-alkylcarbonyl, C₁-C₈-alkoxycarbonyl, C₁-C₈-alkylthiocarbonyl or C₁-C₈-alkylsulfonyl, where the aryl moiety is unsubstituted or substituted by one to five R^{a} and each R^{a} is independently halogen, cyano, nitro, C₁-C₈-alkyl, C₁-C₈-haloalkyl, C₁-C₈-alkoxy or C₁-C₈-haloalkoxy;
R¹ is cyano, halogen, nitro, C₁-C₆-alkyl, C₃-C₆-cycloalkyl, C₂-C₆-alkenyl, C₂-C₆-alkynyl, C₁-C₆-haloalkyl, Z—C₁-C₆-alkoxy, Z—C₁-C₄-alkoxy-C₁-C₄-alkoxy, Z—C₁-C₄-alkylthio, Z—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkoxy, S(O)ₙR^{b}, Z-phenoxy or Z-heterocyclyloxy, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};
Z is independently a covalent bond or C₁-C₄-alkylene;
n is independently 0, 1 or 2;
R^{b} is independently C₁-C₈-alkyl, C₂-C₆-alkenyl, C₂-C₆-alkynyl, C₂-C₆-haloalkenyl, C₂-C₆-haloalkynyl or C₁-C₆-haloalkyl;
R^{c} is independently Z—CN, Z—OH, Z—NO₂, Z-halogen, oxo (═O), ═N-R^{d}, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)R^{d}, NRⁱRⁱⁱ, Z-(tri-C₁-C₄-alkyl)silyl, Z-phenyl or S(O)ₙR^{b};
R^{d} is independently hydrogen, OH, C₁-C₈-alkyl, C₁-C₄-haloalkyl, Z—C₃-C₆-cycloalkyl, C₂-C₈-alkenyl, Z—C₅-C₆-cycloalkenyl, C₂-C₈-alkynyl, Z—C₁-C₆-alkoxy, Z—C₁-C₄-haloalkoxy, Z—C₃-C₈-alkenyloxy, Z—C₃-C₈-alkynyloxy, NRⁱRⁱⁱ, C₁-C₆-alkylsulfonyl, Z-(tri-C₁-C₄-alkyl)silyl, Z-phenyl, Z-phenoxy, Z-phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{c};
RⁱRⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, Z—C₁-C₈-haloalkoxy, Z—C(═O)—R^{d}, Z-phenyl, a 3- to 7-membered monocyclic or 9- or 10-membered bicyclic saturated, unsaturated or aromatic heterocycle which contains 1, 2, 3 or 4 heteroatoms selected from the group consisting of O, N and S and which is attached via Z;
Rⁱ and Rⁱⁱ together with the nitrogen atom to which they are attached may also form a 5- or 6-membered monocyclic or 9- or 10-membered bicyclic heterocycle which contains 1, 2, 3 or 4 heteroatoms selected from the group consisting of O, N and S;
A is N or C—R²;
R²,R³ independently of one another are hydrogen, Z-halogen, Z—CN, Z—OH, Z—NO₂, C₁-C₈-alkyl, C₁-C₄-haloalkyl, C₂-C₈-alkenyl, C₂-C₈-alkynyl, C₂-C₈-haloalkenyl, C₂-C₈-haloalkynyl, Z—C₁-C₈-alkoxy, Z—C₁-C₈-haloalkoxy, Z—C₁-C₄-alkoxy-C₁-C₄-alkoxy, Z—C₁-C₄-alkythio, Z—C₁-C₄-alkylthio-C₁-C₄-alkylthio, Z—C₁-C₆-haloalkylthio, C₂-C₆-alkenyloxy, C₂-C₆-alkynyloxy, C₁-C₆-haloalkoxy, C₁-C₄-haloalkoxy-C₁-C₄-alkoxy, Z—C₃-C₁₀-cycloalkyl, O—Z—C₃-C₁₀-cycloalkyl, Z—C(═O)R^{d}, NRⁱRⁱⁱ, Z—NRⁱSO₂Rⁱⁱ, Z-(tri-C₁-C₄-alkyl)silyl, S(O)ₙR^{b}, Z-phenyl, Z¹-phenyl, Z-heterocyclyl or Z¹-heterocyclyl, where heterocyclyl is a 5- or 6-membered monocyclic or 9- or 10-membered bicyclic saturated, partially unsaturated or aromatic heterocycle which contains 1, 2, 3 or 4 heteroatoms selected from the group consisting of O, N and S, where cyclic groups are unsubstituted or partially or fully substituted by R^{c};
R² together with the group attached to the adjacent carbon atom may also form a 5- to 10-membered saturated or partially or fully unsaturated mono- or bicyclic ring which, in addition to carbon atoms, may contain 1, 2 or 3 heteroatoms selected from the group consisting of O, N and S and may be substituted by further groups R^{c};
Z¹ is independently a covalent bond, C₁-C₄-alkyleneoxy, C₁-C₄-oxyalkylene or C₁-C₄-alkyleneoxy-C₁-C₄-alkylene;
R⁴,R⁵,R⁶ independently of one another are hydrogen, halogen or C₁-C₄-alkyl;
R^{x}, R^{y} independently of one another are hydrogen, C₁-C₈-alkyl, C₂-C₅-alkenyl, C₂-C₅-alkynyl, C₁-C₈-haloalkyl, C₁-C₂-alkoxy-C₁-C₂-alkyl or halogen; or R^{x} and R^{y} are together a C₂-C₅-alkylene or C₂-C₅-alkenylene chain and form a 3-, 4-, 5- or 6-membered saturated, partially unsaturated or fully unsaturated monocyclic ring together with the carbon atom they are bonded to, wherein 1 or 2 of any of the CH₂ or CH groups in the C₂-C₅-alkylene or C₂-C₅-alkenylene chain may be replaced by 1 or 2 heteroatoms independently selected from the group consisting of O and S;
where in the groups R¹, R² and R³ and their subsubstituents, the carbon chains and/or the cyclic groups may be partially or fully substituted by groups R^{c}, or an agriculturally suitable salt or N-oxide thereof.

17: The compound according to claim 16 wherein A is CR².

18: The compound according to claim 17 wherein R² is an optionally substituted 5- or 6-membered saturated, partially unsaturated or aromatic heterocycle which contains 1, 2, 3 or 4 heteroatoms selected from the group consisting of O, N and S.

19: The compound according to claim 18 wherein R² is a heterocycle selected from the group consisting of isoxazoline, tetrazolone, 1,2-dihydrotetrazolone, 1,4-dihydrotetrazolone, tetrahydrofuran, dioxolane, piperidine, morpholine, piperazine, isoxazole, pyrazole, thiazole, oxazole, furyl, pyridine and pyrazine, said heterocycle optionally being substituted with R^{c}, where R^{c} is selected from the group consisting of C₁-C₄-alkyl, C₁-C₄-haloalkyl, C₁-C₄-alkoxy, C₁-C₄-alkoxy-C₁-C₄-alkyl, C₁-C₄-alkylthio-C₁-C₄-alkyl, halogen and oxo.

20: The compound according to claim 17 wherein R² is a group of the formula 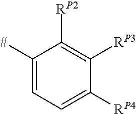
wherein # denotes the bond through which the group R² is attached and
R^{P2} is H or F;
R^{P3} is H, F, Cl or OCH₃; and
R^{P4} is H, F, Cl, CH₃, CF₃, OCH₃, OCH₂OCH₃ or OCH₂CH₂OCH₃.

21. The compound according to claim 17 wherein
R² is an aliphatic group selected from the group consisting of C₁-C₆-alkyl, C₁-C₄-alkoxy-C₁-C₄-alkyl, C₁-C₄-haloalkoxy-C₁-C₄-alkyl, C₂-C₆-alkenyl, C₂-C₆-alkynyl, C₂-C₄-alkoxy, C₂-C₄-haloalkoxy, C₃-C₆-alkenyloxy, C₃-C₆-alkynyloxy, C₃-C₆-haloalkenyloxy, C₃-C₆-haloalkynyloxy, C₁-C₄-alkoxycarbonyl, S(O)₂—C₁-C₈-alkyl, S(O)₂—C₁-C₈-haloalkyl and N—(C₁-C₄-alkyl)amino-N-sulfonyl-C₁-C₄-alkyl.

22. The compound according to claim 16 wherein
R¹ is halogen, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl; and
R³ is H, halogen, CN, NO₂, C₁-C₄-alkyl, C₁-C₄-haloalkyl, C₁-C₄-alkoxy, C₁-C₄-haloalkoxy, C₁-C₄-alkylthio or C₁-C₄-alkylsulfonyl.

23. The compound according to claim 17 wherein R² together with R¹ or R³ forms a 5- to 10-membered mono- or bicyclic saturated, partially unsaturated or fully unsaturated ring which contains 1, 2, 3 or 4 heteroatoms selected from the group consisting of O, N and S, said ring optionally being substituted with R^{b}.

24. The compound according to claim 17 wherein the groups R¹, R² and R³ together form a substitution pattern selected from the group consisting of:
R¹═C₁, R²═H, R³═Cl;
R¹═C₁, R²═H, R³═CF₃;
R¹═C₁, R²═H, R³═SO₂CH₃;
R¹═C₁, R²═H, R³═OCH₃;
R¹═Cl, R²═H, R³═CH₃;
R¹═CH₃, R²═H, R³═Cl;
R¹═CH₃, R²═H, R³═CF₃;
R¹═CH₃, R²═H, R³═SO₂CH₃;
R═CH₃, R²═H, R³═OCH₃;
R¹═CF₃, R²═H, R³═CH₃;
R¹═CF₃, R²═H, R³═Cl;
R¹═CF₃, R²═H, R³═CF₃;
R¹═CF₃, R²═H, R³═SO₂CH₃;
R¹═CF₃, R²═H, R³═OCH₃;
R¹═SO₂CH₃, R²═H, R³═CH₃,
R¹═SO₂CH₃, R²═H, R³═Cl;
R¹═SO₂CH₃, R²═H, R³═CF₃;
R¹═SO₂CH₃, R²═H, R³═SO₂CH₃;
R¹═SO₂CH₃, R²═H, R³═OCH₃; and
R¹═SO₂CH₃, R²═H, R³═CH₃.

25: The compound according to claim 16 wherein A is N.

26: The compound according to claim 25 wherein
R¹ is halogen, nitro, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio or C₁-C₄-alkylsulfonyl; and
R³ is H, halogen CN, nitro, C₁-C₄-alkyl, C₁-C₄-haloalkyl, C₁-C₄-alkoxy, C₁-C₄-alkylthio or C₁-C₄-alkylsulfonyl.

27: The compound according to claim 16 wherein R⁴, R⁵ and R⁶ are H.

28: The compound according to claim 16 wherein R^{x} and R^{y} independently of one another are H or C₁-C₅-alkyl.

29: A composition comprising a herbicidally effective amount of at least one compound of the formula I or an agriculturally suitable salt or N-oxide thereof as defined in claim 16 and auxiliaries customary for formulating crop protection agents.

30: A method for controlling unwanted vegetation which comprises allowing a herbicidally effective amount of at least one compound of the formula I or of an agriculturally suitable salt or N-oxide thereof as defined in claim 16 to act on plants, their seed and/or their habitat.

31: A method for controlling unwanted vegetation which comprises allowing a herbicidally effective amount of at least one compound of the formula I or of an agriculturally suitable salt or N-oxide thereof as defined in claim 17 to act on plants, their seed and/or their habitat.

32: A method for controlling unwanted vegetation which comprises allowing a herbicidally effective amount of at least one compound of the formula I or of an agriculturally suitable salt or N-oxide thereof as defined in claim 18 to act on plants, their seed and/or their habitat.

33: A method for controlling unwanted vegetation which comprises allowing a herbicidally effective amount of at least one compound of the formula I or of an agriculturally suitable salt or N-oxide thereof as defined in claim 19 to act on plants, their seed and/or their habitat.

34: A method for controlling unwanted vegetation which comprises allowing a herbicidally effective amount of at least one compound of the formula I or of an agriculturally suitable salt or N-oxide thereof as defined in claim 20 to act on plants, their seed and/or their habitat.

35: A method for controlling unwanted vegetation which comprises allowing a herbicidally effective amount of at least one compound of the formula I or of an agriculturally suitable salt or N-oxide thereof as defined in claim 21 to act on plants, their seed and/or their habitat.
